(19) United States
(12) Patent Application Publication
(10) Pub. No.: US 2025/0197379 A1
(43) Pub. Date: Jun. 19, 2025

(54) NOVEL RAS INHIBITORS

(71) Applicant: KHR Biotec GmbH, Hamburg (DE)

(72) Inventor: Krishnaraj Rajalingam, Nieder-Olm (DE)

(21) Appl. No.: 18/686,907

(22) PCT Filed: Aug. 26, 2022
(86) PCT No.: PCT/EP2022/025396

§ 371 Date: Feb. 27, 2024

(30) Foreign Application Priority Data
Sep. 1, 2021 (EP) 21020437.6

(51) Int. Cl. C07D 405/12; A61K 31/343; A61K 31/397; A61K 31/4025; A61K 31/4525; A61K 31/496; A61K 31/5377; A61K 31/541; A61K 31/55; C07D 307/93; C07D 413/12; C07D 417/12; C07D 487/08; C07D 491/107

(52) U.S. Cl. CPC C07D 405/12; A61K 31/343; A61K 31/397; A61K 31/4025; A61K 31/4525; A61K 31/496; A61K 31/5377; A61K 31/541; A61K 31/55; C07D 307/93; C07D 413/12; C07D 417/12; C07D 487/08; C07D 491/107

(57) ABSTRACT

The present invention relates to novel compounds and their use as a medicament, in particular for use in treating proliferative disorders. The present invention relates further to a pharmaceutical composition comprising the novel compounds. Moreover, the present invention relates to a method of inhibiting proliferation or metastasis of cancer cells or inducing their cell death in a subject in need thereof. In addition, the present invention relates to a method of inhibiting proliferation of a cell population sensitive towards inhibiting RAS, in particular KRAS, HRAS and NRAS, activation in vitro. Furthermore, the present invention relates to a method of inhibiting proliferation of a cell population sensitive towards inhibiting eIF4A complex in vitro. Furthermore, the present invention relates to a kit containing a formulation comprising a pharmaceutical composition comprising a compound according to the invention.

The present invention relates to novel compounds and their use as a medicament, in particular for use in treating proliferative disorders. The present invention relates further to a pharmaceutical composition comprising the novel compounds. Moreover, the present invention relates to a method of inhibiting proliferation or metastasis of cancer cells or inducing their cell death in a subject in need thereof. In addition, the present invention relates to a method of inhibiting proliferation of a cell population sensitive towards inhibiting RAS, in particular KRAS, HRAS and NRAS, activation in vitro. Furthermore, the present invention relates to a method of inhibiting proliferation of a cell population sensitive towards inhibiting eIF4A complex or engaging PHB1/2 complex in the plasma membrane in vitro. Furthermore, the present invention relates to a kit containing a formulation comprising a pharmaceutical composition comprising a compound according to the invention.

### BACKGROUND OF THE INVENTION

Several approaches are known for the treatment of tumor diseases. A first possibility is the inhibition of RAS proteins, one of the major oncogenes which are mutationally activated in a large section of human cancers.

The RAS oncogenes are frequently mutated in human cancers and among the three isoforms (KRAS, HRAS and NRAS), KRAS is the most frequently mutated oncogene. It is known that some of flavaglines like rocaglamide, a class of natural anti-tumour drugs and chemical ligands of prohibitins, inhibit RAS activation in cells by uncoupling the interaction between RAS and its effectors in the plane of the plasma membrane. Although a treatment with rocaglamide inhibits RAS-activation in KRAS-mutated cell lines, there is still a demand for compounds with a better activity towards RAS oncogenes.

It is also known that various flavagline derivatives exhibit cytotoxic properties. WO 2005/113529 A2 describes cyclopenta[b]benzofuran derivatives and their utilization for the production of medicaments, especially for the prophylaxis and/or therapy of acute or chronic diseases.

WO 2010/060891 A1 describes rocaglaol derivatives and the use of these derivatives to prevent or to limit the cardiotoxicity of an anti-neoplastic agent.

WO 2012/0666002 A1 describes flavagline derivatives and their use as neuroprotective and/or cardioprotective and/or anti-tumor agents.

WO 2017/058768 A1 describes compounds having activity as inhibitors of G12C mutant KRAS proteins.

WO 2020/078975 A1 relates to inhibitors of KRAS oncogene activation, which are flavagline derivatives with the ability to target prohibitin to inhibit KRAS activation.

N. Ribeiro et al., J. Med. Chem., 2012, 55, 100064 relates to flavagline derivates, in particular FL3 and FL23 which are effective in inhibition of cell proliferation and enhancement of viability at lower doses compared to rocaglalol.

Furthermore, it is known that patients frequently develop resistance to RAS oncogene inhibitors, like KRAS G12 C inhibitors (Tanaka et al., Cancer Discov, 2021, PMID 33824136). In addition, the patients treated with KRAS G12 C inhibitors often develop secondary mutations in other RAS isoforms (Awad M M et al. New England J. of Med., 2021, PMID34161704).

As mentioned before, there are already some approaches to inhibit RAS oncogenes. However, effective targeting of this gene with small molecules is still a challenge

Another option for the treatment of tumor diseases is the inhibition of dysregulation of protein translation. In other words, there are some developments targeting eukaryotic initiation factor (eIF4A complex) that integrates multiple oncogenic signaling inputs to the translation apparatus.

Eukaryotic initiation factor 4A (eIF4A) is a DEAD-box protein containing ATPase and ATP-dependent RNA helicase required to melt local secondary structure and facilitate access of the ribosome to the mRNA template. The factor regulates the cap-dependent protein synthesis.

In mammals, there are three isoforms of eIF4A (eIF4AI, II and III), wherein eIF4AII and eIF4AIII share ˜90% and ˜65% identity, respectively, with the most abundant cellular factor eIF4AI. AII isoforms are DEAD-box RNA helicase family members but only the paralogs eIF4AI and eIF4AII are found in the eIF4F complex and participate in translation initiation.

WO 2017/091585 describes compounds having activity as inhibitors of eIF4A. However, the disclosed compounds have a different structure compared to the compounds according to the present invention.

Prohibitins are evolutionarily conserved proteins and recent studies revealed a critical role for prohibitins in the activation of RAS by enabling RAS-effector interaction in the plane of the plasma membrane. Polier et al, Chemistry and Biology, 19, 1093-1104, 2012 showed that rocaglamides target this interaction (PHB1-CRAF) to inhibit RAS-CRAF interaction. These are several follow up studies confirming these effects.

Ernst et al, J. Med. Chem. 2020, 63, 5879 describes that the flavagline compounds rocaglamide A and Zotatifin show inhibition properties towards protein synthesis by stabilizing a translation-incompetent complex for selecting messenger RNAs (mRNAs) with eIF4A.

Accordingly, while advances have been made in this field there remains a significant need for compounds that specifically inhibit RAS and eFI4A activity, particularly with regard to eIF4A's role in regulation of cancer pathways, as well as for associated compositions and methods.

In contrast to the inhibition of RAS oncogenes, which takes place at the plasma membrane possibly by engaging prohibitins driven nanoclusters among others (H. Yurugi er. Al, Journal of Cell Science, 133, 1, 2020), the inhibition of the eukaryotic factor 4A occurs in the cytosol. Therefore, two different oncogenic protein complexes are possibly targeted for profound inhibition of the tumour cell proliferation and survival. Further the activated RAS can also influence of the functioning of the eIF4A complexes through one of the effector pathways (MAPK cascades).

As mentioned before, there are already some approaches to inhibit RAS oncogenes or to inhibit eFI4A activity. However, effective targeting of this genes with small molecules is still a challenge.

It is therefore the object of the present invention to provide pharmaceutically active compounds that have the capability to inhibit the activation of RAS oncogenes, in particular in cells at nanomolar concentrations with high specificity. Furthermore, it is the object of the present invention to provide pharmaceutically active compounds that have the capability to inhibit the RAS and eFI4A activity, in particular in cells at nanomolar concentrations with high specificity, especially, with regard to both RAS's and eIF4A's role in regulation of cancer pathways.

This object is achieved by the compounds of formula (I).

### SUMMARY OF THE INVENTION

The invention relates to a compound of formula (I)

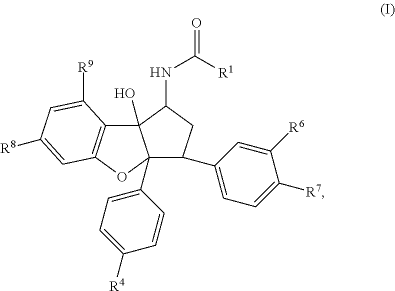

or a pharmaceutically acceptable salt thereof, wherein R¹ is selected from
   C₁-C₄ alkyl, wherein alkyl is unsubstituted or substituted by 1, 2 or 3 substituents R^{a};
   C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom;
   NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
   R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
   R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f};
   R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g};
R^{a} is selected from halogen, OH, C₃-C₇ cycloalkyl, C₁-C₃ alkoxy, phenyl, NR^{5a}R^{5b}, C₁-C₄-alkylsulfonyl, C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b};
R⁹ is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
with the proviso that the following compounds are excluded:
R¹ is NH₂, R⁴ is Cl, R⁶ is F, R⁷ is hydrogen, R⁸ is OCH₃, R⁹ is OCH₃,
R¹ is N(CH₃)₂, R⁴ is Cl, R⁶ is F, R⁷ is hydrogen, R⁸ is OCH₃, R⁹ is OCH₃,
R¹ is CH₂N(CH₃)₂, R⁴ is Cl, R⁶ is F, R⁷ is hydrogen, R⁸ is OCH₃, R⁹ is OCH₃.

The invention further relates to a compound of formula (I-A)

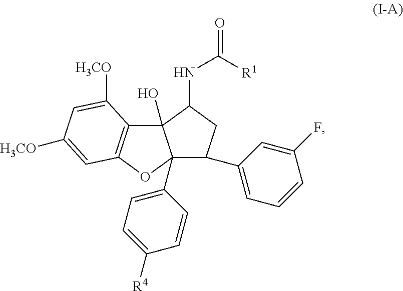

or a pharmaceutically acceptable salt thereof, wherein R¹ is selected from
C₁-C₄ alkyl, wherein alkyl is unsubstituted or substituted by 1, 2 or 3 substituents R^{a};
NR²R³, wherein R² and R³ independently from each other are selected from hydrogen, C₁-C₄ alkyl and C₃-C₇ cycloalkyl, wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they attached to form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
R^{a} is selected from halogen, OH, C₃-C₇ cycloalkyl, C₁-C₃ alkoxy and phenyl, wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
with the proviso that if R⁴ is Cl, R² and R³ are not both hydrogen and are not both methyl, preferably with the proviso that R² and R³ are not both hydrogen and are not both methyl.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use as a medicament.

The invention further relates to a compound (I) according to the invention, in particular to a compound of formulae (I.a), (I.b), (A), (B), (C), (D), (E), (F) and (G) as defined above and below, or a pharmaceutically acceptable salt thereof, or pharmaceutical compositions comprising at least one compound of formulae (I), (I.a), (I.b), (A), (B), (C), (D), (E), (F) and (G), as defined above and below, for use as a medicament.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in the treatment and/or prophylaxis of diseases.

The invention further relates to a compound (I) according to the invention, in particular to a compound of formulae (I.a), (I.b), (A), (B), (C), (D), (E), (F) and (G), as defined above and below, or a pharmaceutically acceptable salt thereof, or pharmaceutical compositions comprising at least one compound of formulae (I), (I.a), (I.b), (A), (B), (C), (D), (E), (F) and (G) as defined above and below, for the treatment and/or prophylaxis of diseases.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in treating proliferative disorders.

The invention further relates to a compound (I) according to the invention, in particular to a compound of formulae (I.a), (I.b), (A), (B), (C), (D), (E), (F) and (G) as defined above and below, or a pharmaceutically acceptable salt thereof, or pharmaceutical compositions comprising at least one compound of formulae (I), (I.a), (I.b), (A), (B), (C), (D), (E), (F) and (G) as defined above and below, for use in treating proliferative disorders.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in treating cancer.

The invention further relates to a compound (I) according to the invention, in particular to a compound of formulae (I.a), (I.b), (A), (B), (C), (D), (E), (F) and (G) as defined above and below or a pharmaceutically acceptable salt thereof, or pharmaceutical compositions comprising at least one compound of formulae (I), (I.a), (I.b), (A), (B), (C), (D), (E), (F) and (G) as defined above and below, for use in treating cancer.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use as inhibitor of RAS protein activation.

The invention further relates to a compound (I) according to the invention, in particular to a compound of formulae (I.a), (I.b), (A), (B), (C), (D), (E), (F) and (G), as defined above and below or a pharmaceutically acceptable salt thereof, or pharmaceutical compositions comprising at least one compound of formulae (I), (I.a), (I.b), (A), (B), (C), (D), (E), (F) and (G), as defined above or below for use as inhibitor of RAS protein (RAS oncogene) activation.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for treating or preventing any diseases or conditions that are associated with the activity of RAS protein (RAS oncogene).

The invention further relates to a compound (I) according to the invention, in particular to a compound of formulae (I.a), (I.b), (A), (B), (C), (D), (E), (F) and (G) as defined above and below or a pharmaceutically acceptable salt thereof, or pharmaceutical compositions comprising at least one compound of formulae (I), (I.a), (I.b), (A), (B), (C), (D), (E), (F) and (G) as defined above or below for treating or preventing any diseases or conditions that are associated with the activity of RAS protein (RAS oncogene).

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in treating proliferative disorders, wherein RAS-signaling is involved, preferably wherein KRAS G12V, NRAS G12V, HRAS G12V, KRAS G12C, KRAS G12D, KRAS G12C/Y96D, KRAS G13C, KRAS G13D, KRASG13S, KRAS Q61H, KRAS Q61R or KRAS Q61 K or wherein any activating mutation in KRAS, HRAS and NRAS is involved or wherein any mutation that acquires resistance to RAS inhibitors.

The invention further relates to a compound (I) according to the invention, in particular to a compound of formulae (I.a), (I.b), (A), (B), (C), (D), (E), (F) and (G) as defined above and below or a pharmaceutically acceptable salt thereof, or pharmaceutical compositions comprising at least one compound of formulae (I), (I.a), (I.b), (A), (B), (C), (D), (E), (F) and (G), as defined above for use in treating proliferative disorders, wherein RAS-signalling is involved, preferably wherein KRAS G12V, NRAS G12V, HRAS G12V, KRAS G12C, KRAS G12D, KRAS G12C/Y96D, KRAS G13C, KRAS G13D, KRASG13S, KRAS Q61H, KRAS Q61R or KRAS Q61 K is involved.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use as inhibitor of eukaryotic ignition factor 4A (eIF4A).

The invention further relates to a compound (I) according to the invention, in particular to a compound of formulae (I.a), (I.b), (A), (B), (C), (D), (E), (F) and (G), as defined above and below or a pharmaceutically acceptable salt thereof, or pharmaceutical compositions comprising at least one compound of formulae (I), (I.a), (I.b), (A), (B), (C), (D), (E), (F) and (G) as defined above or below for use as inhibitor of eukaryotic initiation factor 4A (eIF4A).

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use as a ligand of Prohibitins (PHB1/2 complex) in the plasma membrane.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in treating proliferative disorders, wherein dysregulation of eIF4A is involved, preferably wherein EIF4A1, EIF4A2 or EIF4A3 is involved.

The invention further relates to a compound (I) according to the invention, in particular to a compound of formulae (I.a), (I.b), (A), (B), (C), (D), (E), (F) and (G), as defined above and below or a pharmaceutically acceptable salt thereof, or pharmaceutical compositions comprising at least one compound of formulae (I), (I.a), (I.b), (A), (B), (C), (D), (E), (F) and (G), as defined above or below for treating or preventing any diseases or conditions, wherein dysregulation of eIF4A is involved, preferably wherein eIF4AI, eIF4AII or eIF4AIII is involved.

The invention further relates to a compound of formulae (I), (I-A), (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in treating proliferative disorders, wherein overexpression of Prohibitins (PHB/2) is involved.

The invention further relates to a pharmaceutical composition comprising at least one compound of formula (I), (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, and a pharmaceutically acceptable carrier.

The invention further relates to a pharmaceutical composition comprising at least one compound of formula (I) according to the invention, in particular at least one compound selected from formulae (I.a), (I.b), (A), (B), (C), (D), (E), (F) and (G), as defined above and below, or a pharmaceutically acceptable salt thereof, and a pharmaceutically acceptable carrier.

The invention further relates to a pharmaceutical composition comprising at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, wherein the pharmaceutical composition additionally comprises a further active substance, preferably selected from chemotherapeutic agents, radiotherapeutic agents, immuno-oncology agents and combinations thereof.

The invention further relates to a pharmaceutical composition comprising at least one compound (I) according to the invention, in particular at least one compound selected from formulae (I.a), (I.b), (A), (B), (C), (D), (E), (F) and (G), as defined above and below, or a pharmaceutically acceptable salt thereof, wherein the pharmaceutical composition additionally comprises a further active substance, preferably selected from chemotherapeutic agents, radiotherapeutic agents, immuno-oncology agents and combinations thereof.

The invention further relates to a pharmaceutical composition as defined above and below for use in the prophylaxis and/or treatment of proliferative disorders.

The invention further relates to a pharmaceutical composition comprising at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in the prophylaxis and/or treatment of proliferative disorders.

The invention further relates to a pharmaceutical composition comprising at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, for use in the prophylaxis and/or treatment of genetic disorders where RAS signaling is involved in particular including RASopathies, craniofacial syndrome and Neurofibromatosis type 1.

The invention further relates to a method of inhibiting growth, proliferation, or metastasis of cancer cells in a subject in need thereof, said method comprising administering to the subject a therapeutically effective amount of at least one compound of formula (I), (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt, or a pharmaceutical composition as defined above or below.

The invention further relates to a method of inhibiting growth, proliferation, or metastasis of cancer cells in a subject in need thereof, said method comprising administering to the subject a therapeutically effective amount of at least one compound (I) according to the invention, in particular at least one compound selected from formulae (I.a), (I.b), (A), (B), (C), (D), (E), (F) and (G), as defined above and below, or a therapeutically acceptable salt thereof, or a pharmaceutical composition comprising at least one compound of formulae (I), (I.a), (I.b), (A), (B), (C), (D), (E), (F) and (G) as defined above.

The invention further relates to a method of inhibiting proliferation of a cell population sensitive towards inhibiting RAS activation, in particular inhibiting KRAS, HRAS or NRAS activation in vitro or ex vivo, the method comprising contacting the cell population with at least one compound of formula (I), (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt or a pharmaceutical composition as defined above and below.

The invention further relates to a method of inhibiting proliferation of a cell population sensitive towards inhibiting RAS activation, in particular inhibiting KRAS, HRAS or NRAS activation in vitro or ex vivo, the method comprising contacting the cell population with at least one compound (I) according to the invention, in particular at least one compound selected from formulae (I.a), (I.b), (A), (B), (C), (D), (E), (F) and (G), as defined above and below, or a therapeutically acceptable salt thereof or a pharmaceutical composition comprising at least one compound of formulae (I), (I.a), (I.b), (A), (B), (C), (D), (E), (F) and (G) as defined above.

The invention further relates to method of inhibiting proliferation of a cell population sensitive towards inhibiting eIF4A in vitro or ex vivo, the method comprising contacting the cell population with at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt a pharmaceutical composition as defined above and below.

The invention further relates in particular to method of inhibiting proliferation of a cell population sensitive towards inhibiting eIF4A in vitro or ex vivo and thus the downstream signaling thereof, the method comprising contacting the cell population with at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt a pharmaceutical composition as defined above and below.

The invention further relates to a method of inhibiting proliferation of a cell population sensitive towards inhibiting eIF4A activation in vitro or ex vivo, the method comprising contacting the cell population with at least one compound (I) according to the invention, in particular at least one compound selected from formulae (I.a), (I.b), (A), (B), (C), (D), (E), (F) and (G), as defined above and below, or a therapeutically acceptable salt thereof or a pharmaceutical composition comprising at least one compound of formulae (I), (I.a), (I.b), (A), (B), (C), (D), (E), (F) and (G) as defined above.

The invention further relates to a kit comprising formulation comprising: a1) at least one compound of formula (I)(I) or (I-A), in particular a compound selected from formulae (I.a), (I.b)(I.a′), (A), (B), (C), (D), (E), (F) (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt or a2) a pharmaceutical composition comprising at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable, or a therapeutically acceptable salt thereof and a pharmaceutically acceptable carrier; and b) instructions for dosing of the pharmaceutical composition for the treatment of a disorder in which inhibition of RAS activation is effective in treating the disorder.

The invention further relates to a kit comprising formulation comprising: a1) at least one compound of formula (I)(I) or (I-A), in particular a compound selected from formulae (I.a), (I.b)(I.a′), (A), (B), (C), (D), (E), (F) (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt as defined above and below, an enantiomeric mixture thereof, wherein each of the compounds can be in the form of a pharmaceutically acceptable, or a therapeutically acceptable salt thereof and a pharmaceutically acceptable carrier; and b) instructions for dosing of the pharmaceutical composition for the treatment of a disorder in which inhibition of RAS activation is effective in treating the disorder.

The invention further relates to a kit comprising formulation comprising: a2) a pharmaceutical composition comprising at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable, or a therapeutically acceptable salt thereof and a pharmaceutically acceptable carrier; and b) instructions for dosing of the pharmaceutical composition for the treatment of a disorder in which inhibition of RAS activation is effective in treating the disorder.

The invention further relates to a kit containing a formulation comprising: a) at least compound (I) according to the invention, in particular at least compound selected from formulae (I.a), (I.b), (A), (B), (C), (D), (E), (F) and (G), as defined above and below, or a pharmaceutical composition comprising at least one compound (I) according to the invention, in particular at least one compound selected from formulae (I.a), (I.b), (A), (B), (C), (D), (E), (F) and (G) as defined above or below, or a therapeutically acceptable salt thereof and a pharmaceutically acceptable carrier; and b) instructions for dosing of the pharmaceutical composition for the treatment of a disorder in which inhibition of RAS activation is effective in treating the disorder.

The invention further relates to a kit comprising: a1) at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T) (U), (V) as defined above and below, an enantiomeric mixture thereof, wherein each of the compounds can be in the form of a pharmaceutically acceptable salt or a2) a pharmaceutical composition comprising at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt and a pharmaceutically acceptable carrier; and b) instructions for dosing of the pharmaceutical composition for the treatment of a disorder in which inhibition of the activity of eIF4A, is effective in treating the disorder.

The invention further relates to a kit comprising: a1) at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T) (U), (V) as defined above and below, an enantiomeric mixture thereof, wherein each of the compounds can be in the form of a pharmaceutically acceptable salt, an enantiomeric mixture thereof, wherein each of the compounds can be in the form of a pharmaceutically acceptable salt and a pharmaceutically acceptable carrier; and b) instructions for dosing of the pharmaceutical composition for the treatment of a disorder in which inhibition of the activity of eIF4A, is effective in treating the disorder.

The invention further relates to a kit comprising a2) a pharmaceutical composition comprising at least one compound of formula (I) or (I-A), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) as defined above and below, an enantiomeric mixture thereof, wherein each of the compounds can be in the form of a pharmaceutically acceptable salt and a pharmaceutically acceptable carrier; and b) instructions for dosing of the pharmaceutical composition for the treatment of a disorder in which inhibition of the activity of eIF4A, is effective in treating the disorder.

The invention further relates to a kit containing a formulation comprising: a) at least one compound according to the invention, in particular at least compound selected from formulae (I.a), (I.b), (A), (B), (C), (D), (E), (F) and (G), as defined above and below, or pharmaceutical composition comprising at least on compound of formula (I), in particular at least compound selected from formulae (I.a), (I.b), (A), (B), (C), (D), (E), (F) and (G), as defined above or a therapeutically acceptable salt thereof and a pharmaceutically acceptable carrier; and b) instructions for dosing of the pharmaceutical composition for the treatment of a disorder in which inhibition of dysregulation of protein translation, wherein eIF4A is involved, is effective in treating the disorder.

The invention further relates to a process for the preparation of a compound of process for the preparation of a compound of the formula (I)

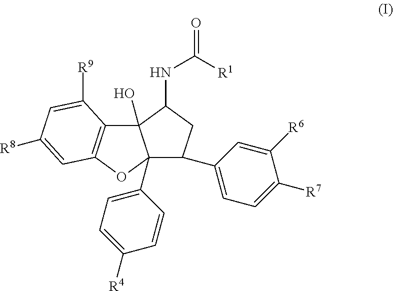

or stereoisomers, tautomers, or pharmaceutically acceptable salts thereof, wherein
R¹ is defined above and below;
R⁴ is selected from Cl, CN and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
comprising the steps
a1) providing a compound of the formula (II)

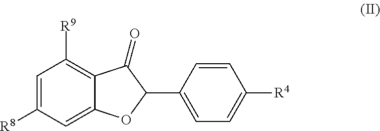

wherein
R⁴ is selected from Cl, CN and C₃-C₇ cycloalkyl,
a2) reacting the compound (II) with a compound (III)

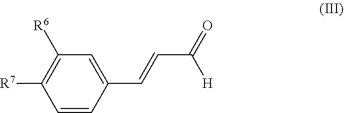

to yield the adduct (IV)

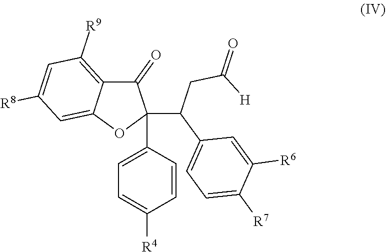

a3) reacting the compound (IV) with trimethylsilylcyanide to give the cyanohydrin silylether (V)

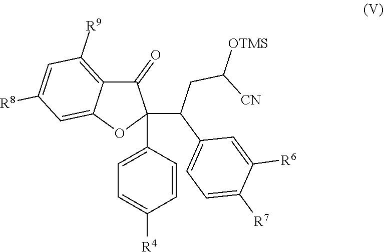

a4) subjecting the compound (V) to a ring formation reaction in the presence of a base to give the compound (VI)

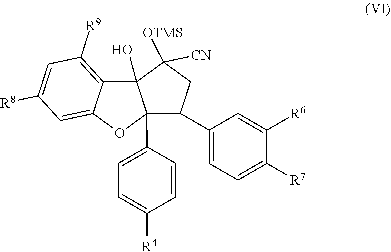

a5) reacting the compound (VI) with tetra-n-butylammonium fluoride to give the compound (VII)

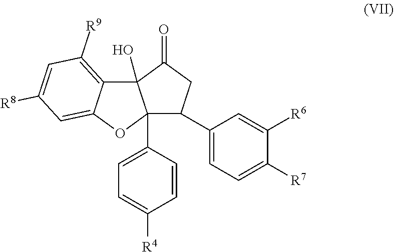

a6) reacting the compound (VII) with methoxyamine hydrochloride to give the oxime compound (VIII)

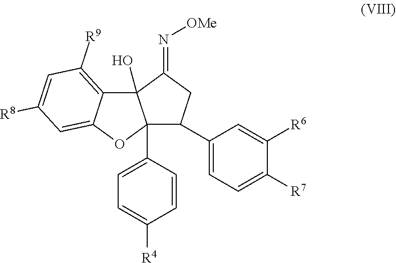

a7) reduction of the oxime compound (VIII) give the amine compound (IX)

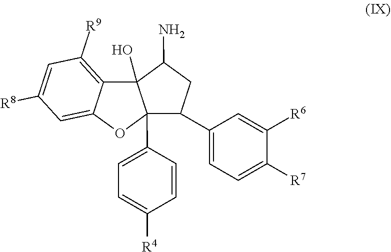

a8.1) subjecting the amine compound (IX) to a reaction with a compound of the formula (X.1)

R¹—C(═O)—X (X.1)
wherein X is a leaving group selected from Cl, Br, O-benzyl, CH₃SO₃ and CF₃SO₃
to give the compound of the formula (I)
or
a8.2) subjecting the amine compound (IX) to a reaction with an isocyanate of the formula (X.2)

R²—N═C═O (X.2)
to give a compound of the formula (I), wherein R¹ is a group NHR², wherein R² is selected from C₁-C₄ alkyl and C₃-C₇ cycloalkyl, wherein alkyl is unsubstituted or substituted by 1, 2 or 3 substituents R^{a}, wherein cycloalkyl is unsubstituted or substituted by 1, 2 or 3 substituents R^{b},
R^{a} is selected from halogen, OH, C₃-C₇ cycloalkyl, C₁-C₃ alkoxy and phenyl, wherein C₃-C₇ cycloalkyl and phenyl is unsubstituted or substituted by 1, 2 or 3 substituents selected from F, Cl, Br and OH,
R^{b} is selected from halogen, OH and C₁-C₃ alkoxy,
a9) optionally subjecting at least one compound, selected from compounds (IV) obtained in step a2), compounds (V) obtained in step a3), compounds (VI) obtained in step a4), compounds (VII) obtained in step a5), compounds (VIII) obtained in step a6), compounds (IX) obtained in step a7), compounds (I) obtained in step a8.1) and compounds (I) obtained in step a8.2), to one or more purification step(s).

The invention further relates to the preparation of a compound of the formula (I)

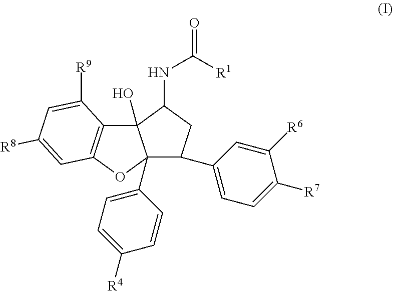

or stereoisomers, tautomers, or pharmaceutically acceptable salts thereof, wherein
R¹ is defined as in any of claims 1, 2, 3, 4, 5, 6, 7, 8, 9 or 10;
R⁴ is CN;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
comprising the steps
a1) providing a compound of the formula (II)

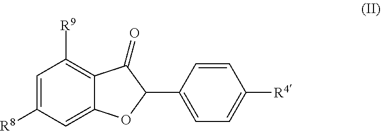

wherein
R^{4′} is selected from halogen, in particular Br,
a2) reacting the compound (II) with a compound (III)

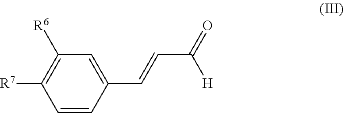

to yield the adduct (IV)

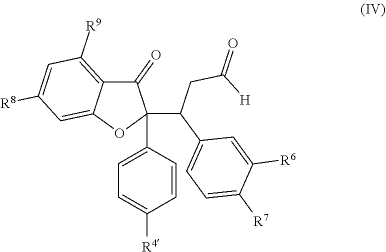

a3) reacting the compound (IV) with trimethylsilylcyanide to give the cyanohydrin silylether (V)

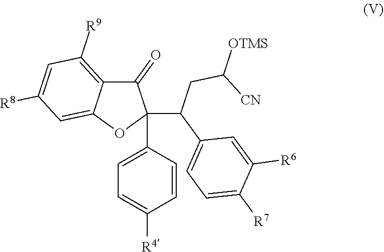

a4) subjecting the compound (V) to a ring formation reaction in the presence of a base to give the compound (VI)

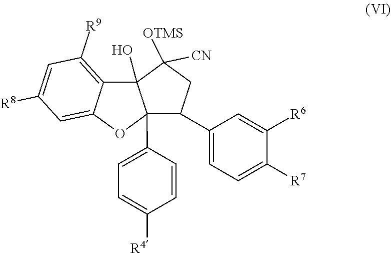

a5) reacting the compound (VI) with tetra-n-butylammonium fluoride to give the compound (VII)

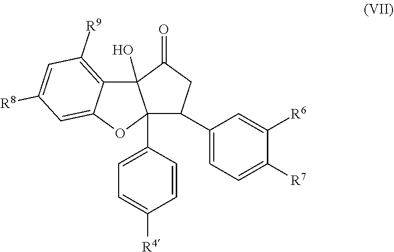

a6) reacting the compound (VII) with methoxyamine hydrochloride to give the oxime compound (VIII)

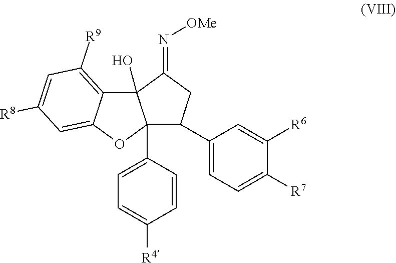

a7) reduction of the oxime compound (VIII) give the amine compound (IX)

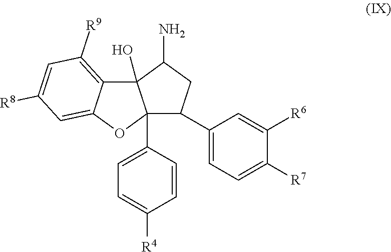

a7.1) reacting the compound (IX) with benzyl carbonochloridate to give the compound (IX′)

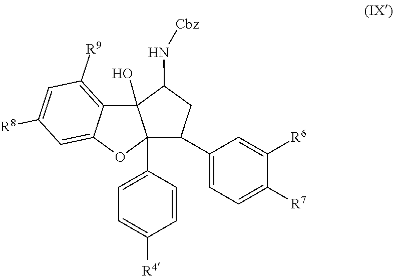

wherein Cbz is benzyloxycarbonyl,
a7.2) reacting the compound (IX′) with dicyanozinc followed by the cleavage of the Cbz group to give the compound (IX″),

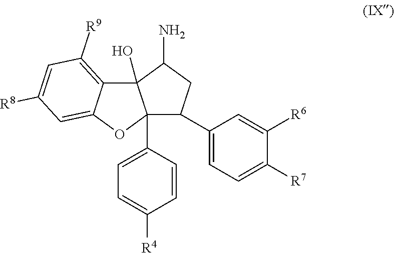

a8.1) subjecting the amine compound (IX″) to a reaction with a compound of the formula (X.1)

R¹—C(═O)—X (X.1)
wherein X is a leaving group selected from Cl, Br, O-benzyl, CH₃SO₃ and CF₃SO₃
to give the compound of the formula (I)
or
a8.2) subjecting the amine compound (IX″) to a reaction with an isocyanate of the formula (X.2)

R²—N═C═O (X.2)
to give a compound of the formula (I), wherein R¹ is a group NHR², wherein R² is selected from C₁-C₄ alkyl and C₃-C₇ cycloalkyl, wherein alkyl is unsubstituted or substituted by 1, 2 or 3 substituents R^{a}, wherein cycloalkyl is unsubstituted or substituted by 1, 2 or 3 substituents R^{b},
R^{a} is selected from halogen, OH, C₃-C₇ cycloalkyl, C₁-C₃ alkoxy and phenyl, wherein C₃-C₇ cycloalkyl and phenyl is unsubstituted or substituted by 1, 2 or 3 substituents selected from F, Cl, Br and OH,
R^{b} is selected from halogen, OH and C₁-C₃ alkoxy,
a9) optionally subjecting at least one compound, selected from compounds (IV) obtained in step a2), compounds (V) obtained in step a3), compounds (VI) obtained in step a4), compounds (VII) obtained in step a5), compounds (VIII) obtained in step a6), compounds (IX) obtained in step a7), compounds (1) obtained in step a8.1) and compounds (1) obtained in step a8.2), to one or more purification step(s).

The invention in particular relates to a process for the preparation of a compound of the formula (I-A)

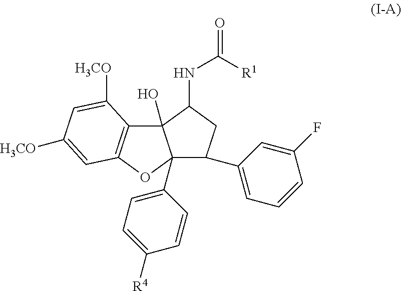

or stereoisomers, tautomers, or pharmaceutically acceptable salts thereof, wherein
R¹ is defined as above and in the following,
R⁴ is selected from Cl, CN and C₃-C₇ cycloalkyl, comprising the steps
a1) providing a compound of the formula (II-A)

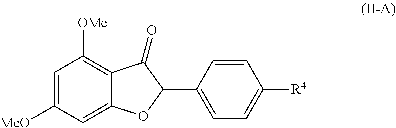

wherein
R⁴ is selected from Cl and C₃-C₇ cycloalkyl,
a2) reacting the compound (II-A) with a compound (III-A)

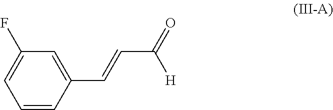 [SMILES: O=C/C=C/c1cccc(F)c1]

to yield the adduct (IV-A)

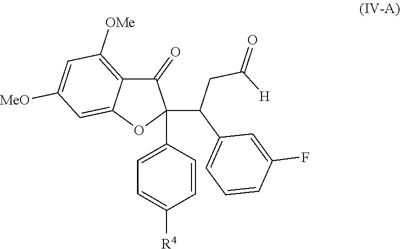

a3) reacting the compound (IV-A) with trimethylsilylcyanide to give the cyanhydrin silylether (V-A)

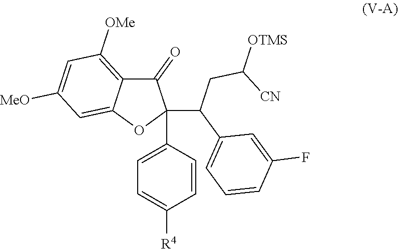

a4) subjecting the compound (V-A) to a ring formation reaction in the presence of a base to give the compound (VI-A)

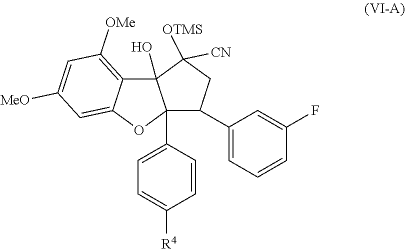

a5) reacting the compound (VI-A) with tetra-n-butylammonium fluoride to give the compound (VII-A)

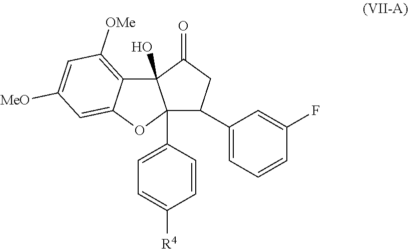

a6) reacting the compound (VII-A) with methoxyamine hydrochloride to give the oxime compound (VIII-A)

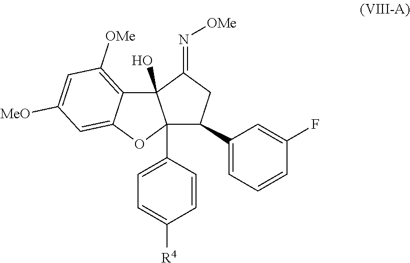

a7) reduction of the oxime compound (VIII-A) give the amine compound (IX-A)

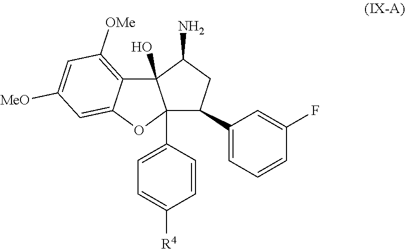

a8.1) subjecting the amine compound (IX-A) to a reaction with a compound of the formula (X.1-A)

R¹—C(═O)—X (X.1-A)
wherein X is a leaving group selected from Cl, Br, O-benzyl, CH₃SO₃ and CF₃SO₃, to give the compound of the formula (I-A)
or
a8.2) subjecting the amine compound (IX)-A to a reaction with an isocyanate of the formula (X.2-A)

R²—N═C═O (X.2-A)
to give a compound of the formula (I), wherein R¹ is a group NHR², wherein R² is selected from C₁-C₄ alkyl and C₃-C₇ cycloalkyl, wherein alkyl is unsubstituted or substituted by 1, 2 or 3 substituents R^{a}, wherein cycloalkyl is unsubstituted or substituted by 1, 2 or 3 substituents R^{b},
R^{a} is selected from halogen, OH, C₃-C₇ cycloalkyl, C₁-C₃ alkoxy and phenyl, wherein C₃-C₇ cycloalkyl and phenyl is unsubstituted or substituted by 1, 2 or 3 substituents selected from F, Cl, Br and OH,
R^{b} is selected from halogen, OH and C₁-C₃ alkoxy,
a9) optionally subjecting at least one compound, selected from compounds (IV-A) obtained in step a2), compounds (V-A) obtained in step a3), compounds (VI-A) obtained in step a4), compounds (VII-A) obtained in step a5), compounds (VIII-A) obtained in step a6), compounds (IX-A) obtained in step a7), compounds (I-A) obtained in step a8.1) and compounds (I-A) obtained in step a8.2), to one or more purification step(s).

### DESCRIPTION OF THE INVENTION

The invention has the following advantages:
The compounds according to the invention exhibit advantageous RAS inhibition properties. In other words the compounds according to the invention qualify as inhibitors of RAS oncogene activation by inhibiting the prohibitin pathway, in particular inhibiting EGF-induced RAS-GTP loading in cells which is measured by the ability of RAS to bind to its effector proteins like RAF kinases.
The compounds according to the invention prevent the activation of RAS, in particular KRAS, as the interaction between RAS, in particular KRAS, and its effectors is uncoupled due to defects in nanoclustering of RAS, in particular KRAS, in the plane of the plasma membrane.
It is possible to identify potential candidate substances, by conducting a screening of compounds according to the invention that are able to inhibit the activation of RAS by directly disrupting the interaction between activated KRAS (both by EGF and mutational activation) and the RAS binding domain (RBD) of the CRAF kinase in cells or other RAS interaction domains like the RA (RAS associating) domain.
The compounds are obtainable on reasonable scale for further development.
The compounds are soluble with good pharmacokinetic/pharmacodynamic (PK/PD) properties. These compounds inhibit KRAS irrespective of the mutations at nanomolar range and also exhibit further inhibitory effects on NRAS and HRAS.
In addition, the compounds, in particular at increasing concentrations and at longer time points post treatment, inhibit eIF4A complex, which is responsible for the translation of several oncogenes. This complex has been targeted by the so called STIs (Selective translational inhibitors).
A dual luciferase assay has been established to measure eIF4A activity in vitro and the compounds are further screened for the inhibition of eIF4A.

### Compounds of Formula (I)

Unless specifically stated otherwise herein, references made in the singular may also include the plural. For example, “a” and “an” may refer to either one, or one or more.

In the context of the invention, the prefix Cn-Cm indicates the number of carbon atoms that a molecule or residue designated thereby may contain.

In the context of the invention, the expression “C₁-C₄-alkyl” refers to unbranched or branched saturated hydrocarbon groups having 1 to 4 carbon atoms. C₁-C₄-alkyl are e.g. methyl, ethyl, propyl, 1-methylethyl, butyl, 1-methylpropyl, 2-methylpropyl, 1,1-dimethylethyl.

In the context of the invention the expression “C₁-C₄-alkoxy” refers to an unbranched or branched saturated C₁-C₄-alkyl group as defined above, which is bound via an oxygen atom. Alkoxy radicals with 1 or 2 carbon atoms are preferred. C₁-C₂-alkoxy is methoxy or ethoxy. C₁-C₄-alkoxy is e.g. methoxy, ethoxy, n-propoxy, 1-methylethoxy (isopropoxy), butoxy, 1-methylpropoxy (sec-butoxy), 2-methylpropoxy (isobutoxy) or 1,1-dimethylethoxy (tert-butoxy).

In the context of the invention the expression “carbonyloxy-C₁-C₄-alkyl” refers to an unbranched or branched saturated C₁-C₄-alkyl group as defined above, which is bound via an carboxyl group.

In the context of the invention the expressions “haloalkyl” and “haloalkoxy” refer to partially or fully halogenated alkyl or alkoxy. In other words, one or more hydrogen atoms, for example 1, 2, 3, 4 or 5 hydrogen atoms bonded to one or more carbon atoms of alkyl or alkoxy are replaced by a halogen atom, in particular by fluorine or chlorine.

In the context of the invention the expressions “hydroxyalkyl” refer to partially or fully hydroxylated alkyl. In other words, one or more hydrogen atoms, for example 1, 2, 3, 4 or 5 hydrogen atoms bonded to one or more carbon atoms of alkyl are replaced by a hydroxy atom.

In the context of the invention the expression “C₃-C₇-cycloalkyl” refers to monocyclic cycloaliphatic radicals having from 3 to 7 carbon atoms, such as cyclopropyl, cyclobutyl, cyclopentyl, cyclohexyl, cycloheptyl, cyclooctyl, cyclononyl and cyclodecyl, preferably cyclopropyl, cyclobutyl, cyclopentyl and cyclohexyl.

The expression “halogen” denotes in each case fluorine, chlorine, bromine or iodine.

In the context of the invention the expression “3-, 4-, 5-, 6- or 7-membered saturated, or partially unsaturated heterocyclic ring containing 1, 2 or 3 heteroatoms or heteroatom containing groups, wherein those heteroatom(s) (group(s)) are selected from N, O, S, NR^{c}, SO and SO₂ are ring members”, refers to monocyclic or polycyclic radicals that are attached to the remainder of the molecule via a nitrogen ring member. “Heterocyclic ring” also in particular comprises “polycyclic”, for example bicyclic, tricyclic or tetracyclic ring systems, in which one of the abovementioned monocyclic heterocylcyl residues is condensed or bridged with at least one further, identical or different heterocyclic ring or, at least one cycloalkyl according to the above definition in each case. Examples of 3-, 4-, 5-, 6- or 7-membered saturated heterocyclic rings include: aziridinyl, azetidinyl, pyrrolidinyl, pyrazolidinyl, pyrazolidinyl, imidazolidinyl, oxazolidinyl, isoxazolidinyl, thiazolidinyl, isothiazolidinyl, 1,2,4-oxadiazolidinyl, 1,2,4-thiadiazolidinyl, 1,2,4 triazolidinyl, 1,3,4-oxadiazolidinyl, 1,3,4 thiadiazolidinyl, 1,3,4 triazolidinyl, piperidinyl, hexahydropyridazinyl, hexahydropyrimidinyl, hexahydropyrimidinyl, piperazinyl, 1,3,5-hexahydrotriazinyl, 1,2,4 hexahydrotriazinyl, morpholinyl, 2-thiomorpholinyl, 3-thiomorpholinyl, 1-oxothiomorpholinyl, 1-oxothiomorpholinyl, 1,1-dioxothiomorpholinyl, 1,1-dioxothiomorpholinyl, hexahydroazepinyl, hexahydrooxepinyl, hexahydrodiazepinyl, hexahydrooxazepinyl and the like. Examples of 3-, 4-, 5-, 6- or 7-membered partially unsaturated heterocyclic rings include: pyrrolinyl, isoxazolinyl, isothiazolinyl, dihydropyrazolyl, tetrahydropyridinyl, tetrahydropyridazinyl, tetrahydropyridazinyl and tetrahydropyrimidinyl. Examples of 3-, 4-, 5-, 6- or 7-membered polycyclic ring system, in particular a bi-, tri- or tetracyclic ring system include: 8-methyl-3,8-diazabicyclo[3.2.1]octan-3-yl.

In the context of the invention the expression “C₃-C₇ heterocyloalkyl” refers to saturated cycloaliphatic groups having 3 to 7, preferably 3 to 6 ring atoms, in which 1, 2 or 3 of the ring carbon atoms have been replaced by heteroatoms or heteroatom-containing groups, preferably selected from NR^{c}, O, S, SO and SO₂ and which may be optionally substituted. In the case of a substitution, these heterocycloaliphatic groups preferably have 1, 2 or 3, particularly preferably 1 or 2, particularly 1 substituent(s). The heterocyloalkyl refers to monocyclic radicals that are attached to the remainder of the molecule via a carbon ring member. By way of example of such heterocycloaliphatic residues, mention may be made of aziridinyl, azetidinyl, pyrrolidinyl, pyrazolidinyl, imidazolidinyl, oxazolidinyl, isoxazolidinyl, thiazolidinyl, isothiazolidinyl, 1,2,4-oxadiazolidinyl, 1,2,4-thiadiazolidinyl, 1,2,4 triazolidinyl, 1,3,4-oxadiazolidinyl, 1,3,4 thiadiazolidinyl, 1,3,4 triazolidinyl, piperidinyl, hexahydropyridazinyl, hexahydropyrimidinyl, hexahydropyrimidinyl, piperazinyl, 1,3,5-hexahydrotriazinyl, 1,2,4 hexahydrotriazinyl, morpholinyl, 2-thiomorpholinyl, 3-thiomorpholinyl, 1-oxothiomorpholinyl, 1-oxothiomorpholinyl, 1,1-dioxothiomorpholinyl, 1,1-dioxothiomorpholinyl, hexahydroazepinyl, hexahydrooxepinyl, hexahydrodiazepinyl, hexahydrooxazepinyl and the like.

In the context of the invention the expression “spiro” refers to compounds, which have at least two molecular rings with only one common atom.

The compounds of formulae (I) or (I-A), in particular of formulae (I.a), (I.a′), (I-A.a), (I-A.a′), (A), (B), (C), (D), (E), (F), (G), (H), (I), (J), (K), (L), (M), (N), (O), (P), (Q), (R), (S), (T), (U), (V) an enantiomeric mixture thereof, may form salts which are also within the scope of this invention. The term “salt(s)” as employed herein, denotes acidic and/or basic salts formed with inorganic and/or organic acids and bases. Pharmaceutically acceptable (i.e. non-toxic, physiologically acceptable) salts are preferred, although other salts are also useful, e.g., in isolation or purification steps which may be employed during preparation. Salts of the compounds of formula (I) or (I-A), in particular of formulae (I.a), (I.a′), (I-A.a), (I-A.a′) (A), (B), (C), (D), (E), (F), (G), (H), (I), (J), (K), (L), (M), (N), (O), (P), (Q), (R), (S), (T), (U), (V) or an enantiomeric mixture thereof may be formed, for example, by reacting a compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) or an enantiomeric mixture thereof with at least one acid or base. The acid or base is added in an amount suitable for partial or complete neutralization, e.g. an equivalent amount.

The phrase “pharmaceutically acceptable salt(s)” as used herein, unless otherwise indicated, includes salts containing pharmacologically acceptable anions or cations, such as chloride, bromide, iodide, nitrate, sulfate, bisulfate, phosphate, hydrogen phosphate, dihydrogen phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, sulfate, benzenesulfonate, p-toluenesulfonate and palmoate [i.e. 4,4′-methylene-bis-(3-hydroxy-2-naphthoate)] salts.

In the context of the present invention, a chemical structure that does not explicitly show a specific stereochemical orientation usually means all possible stereoisomers and mixtures thereof, unless indicated otherwise, for example,

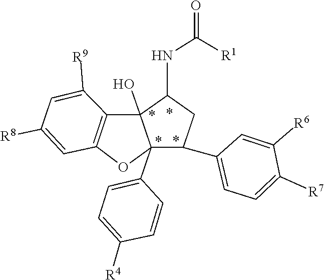

in which * designates the asymmetry centers.

“Chiral compounds” in the sense of the invention are compounds that contain no improper axis of rotation (Sₙ). In the context of the present invention, they are in particular compounds with at least four chirality centers and without Sₙ-symmetry.

“Stereoisomers” in the context of the invention are compounds of identical constitution but different atomic arrangement in the three-dimensional space.

“Enantiomers” are stereoisomers which behave like image to mirror image to one another, e.g. compounds of formulae (I.a) and (I.b) are enantiomers. The “enantiomeric excess” (ee) achieved during asymmetric synthesis is given here by the following formula: ee [%]=(R−S)/(R+S)×100. R and S are the descriptors of the CIP system for the two enantiomers and describe the absolute configuration on the asymmetric atom. The enantiomerically pure compound (ee=100%) is also referred to as “homochiral compound”.

“Diastereomers” are stereoisomers which are not enantiomeric to one another.

The compound of the invention can exist in various isomeric forms, as well as in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, or via chemical separation of stereoisomers through the employment of optically active resolving agents.

Unless otherwise indicated“stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

A“stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

Compounds of the invention or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography.

Relative configuration in stereochemistry (relative stereochemistry) is the arrangement of atoms or groups of atoms that is described relative to other atoms or groups of atoms in the molecule. In other words, this term describes the position of atoms or groups of atoms in space in relation to other atoms or groups of atoms that are located elsewhere in the molecule.

Absolute configuration in stereochemistry (absolute stereochemistry) is the arrangement of atoms or group of atoms that is described independently of any other atom or group of atoms in the molecule. This type of configuration is defined for chiral molecular entities and their stereochemical descriptions (e.g. R or S).

Syn means that with regard to the orientation of the substituents on the 5-membered ring they are bound to (4 asymmetric carbon atoms) all substituents point in the same direction relative to the plane of the 5-membered ring.

The indication (+/−) in the formulae according to the invention indicates that the compounds are present as a racemic mixture.

Racemic mixture or racemate is defined as a mixture of compounds consisting of two molecules structured like image and mirror image (=enantiomers) and which are present in an equimolar mixture, i.e. in the ratio 1:1 (50:50).

Compounds of formulae (I.a′) and (I-A.a′)

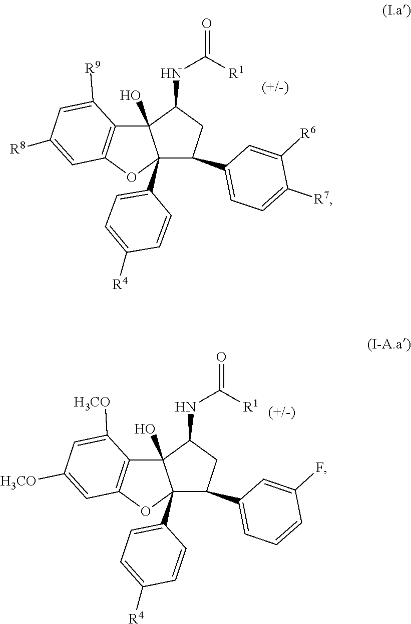

wherein R¹, R⁴, R⁶, R⁷, R⁸ and R⁹ have one of the meanings as defined above or below, relates to compounds, wherein the relative stereochemistry of the compounds is specified.

The compounds of formula (I.a′) and (I-A.a′) have a relative stereochemistry of all syn and are a racemic mixture of two enantiomers (all 4 substituents bound on the 5-membered ring oriented in the same direction).

One preferred embodiment of the invention is a racemic mixture of formula (I.a′).

Another preferred embodiment of the invention is a racemic mixture of formula (I-A.a′).

In other words compounds (I.a′) ((+/−)) relates to a mixture of compounds (I.a) and (I.b), which are depicted below, wherein the ratio of (I.a):(I.b) is 1:1

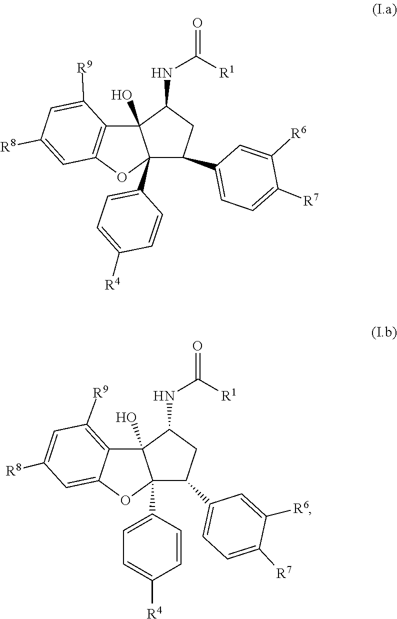

wherein R¹, R⁴, R⁶, R⁷, R⁸ and R⁹ have one of the meanings as defined above or below.

Further, compound (I-A.a′) ((+/−)) relates to a mixture of compounds (I-A.a) and (I-A.b), which are depicted below, wherein the ratio of (I-A.a):(I-A.b) is 1:1

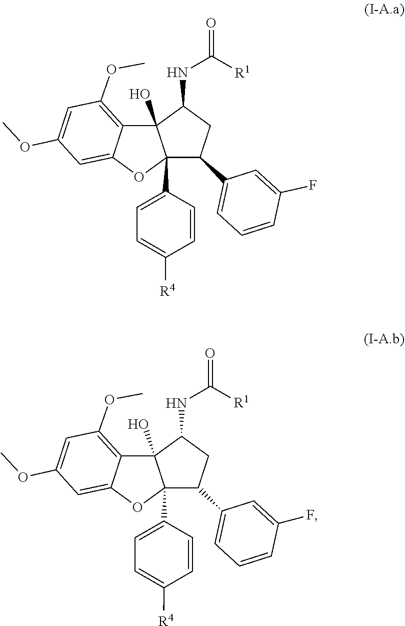

wherein R¹ and R⁴ have one of the meanings as defined above or below.

The compounds of formula (I)

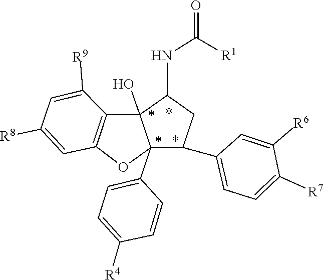

in which * designates the asymmetry centers, represent the isomers of formulae (I.a), (I.b), (I.c), (I.d), (I.e), (I.f), (I.g), (I.h), (I.i), (I.j), (I.k), (I.l), (I.m), (I.n), (I.o) and (I.p):

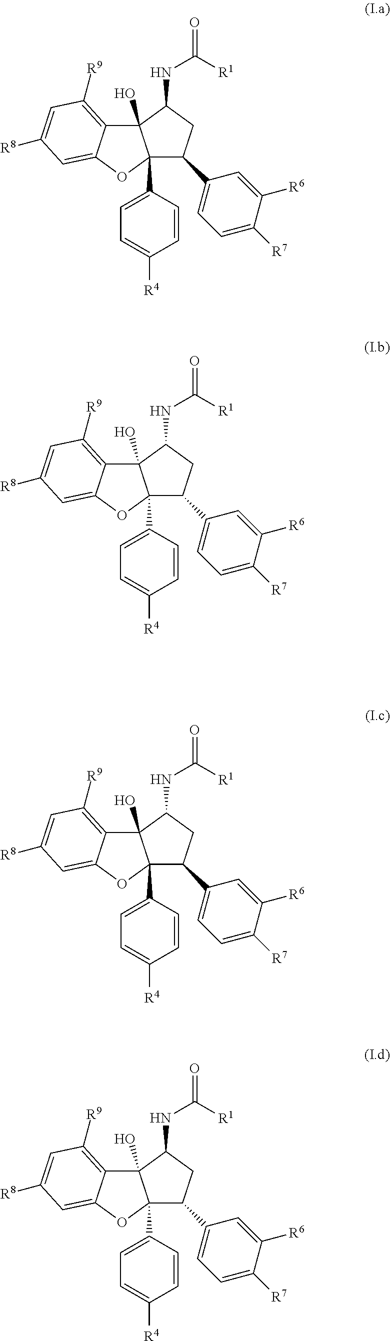

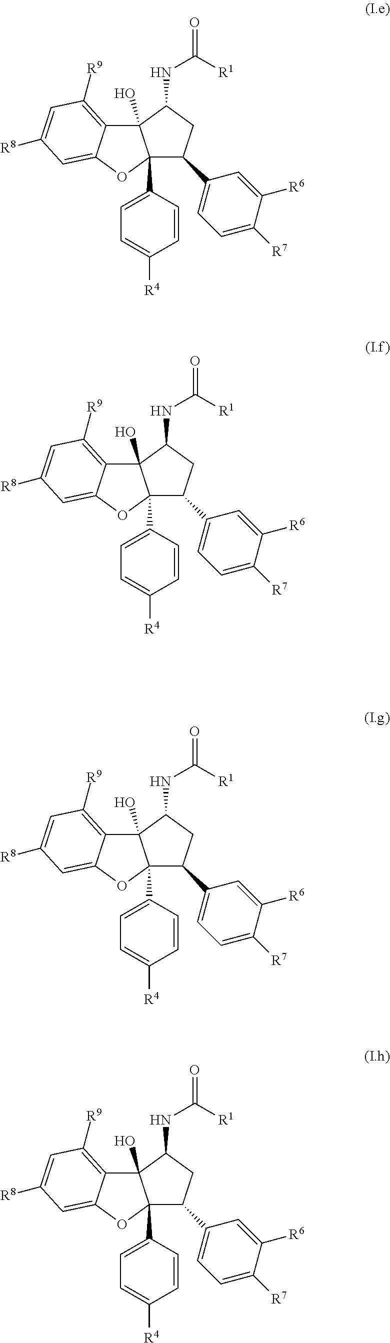

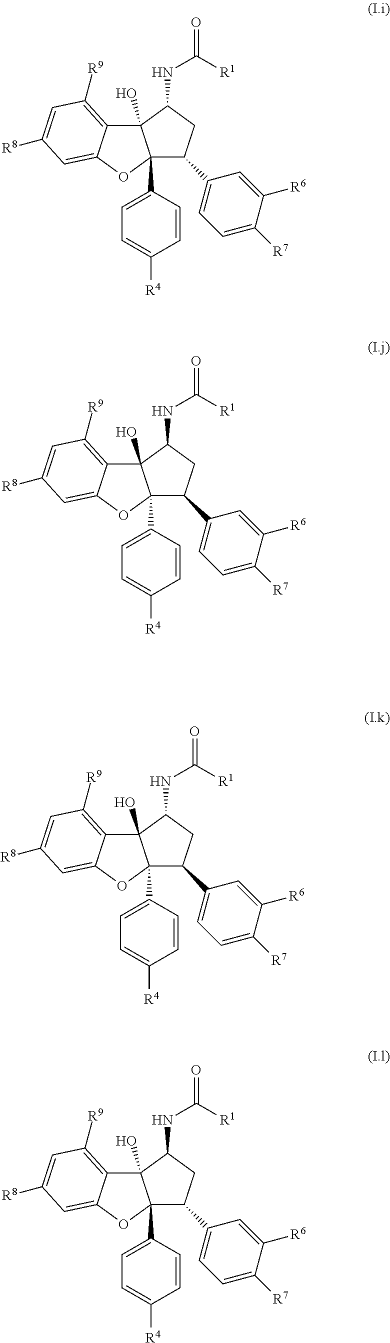

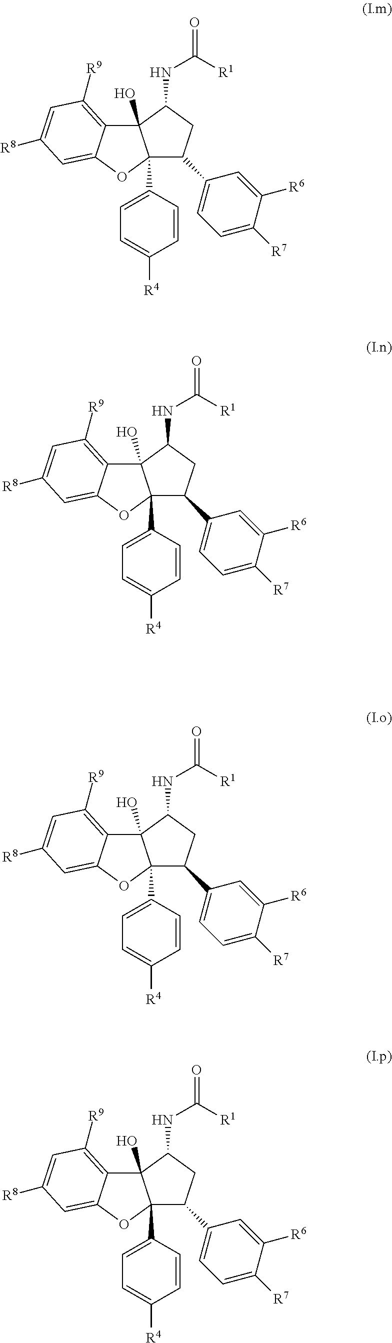

wherein, R¹, R⁴, R⁹, R⁷, R⁸ and R⁹ have one of the meanings as defined above or below. The compounds of formulae (I.a) to (I.p) are specified by their absolute stereochemistry. Further, the compounds of formula (I-A)

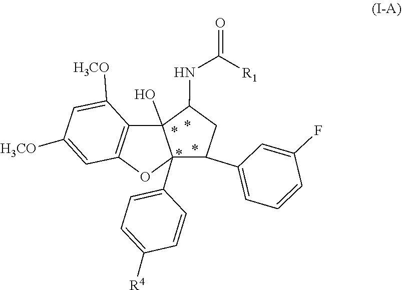

in which * designates the asymmetry centers, represent the isomers of formulae (I-A.a), (I-A.b), (I-A.c), (I-A.d), (I-A.e), (I-A.f), (I-A.g), (I-A.h), (I-A.i), (I-A.j), (I-A.k), (I-A.l), (I-A.m), (I-A.n), (I-A.o) and (I-A.p):

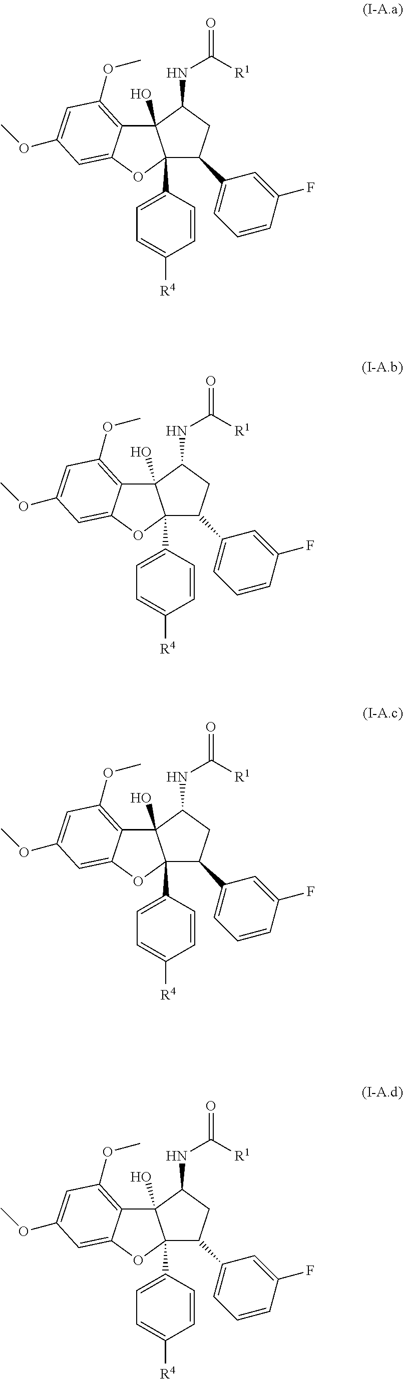

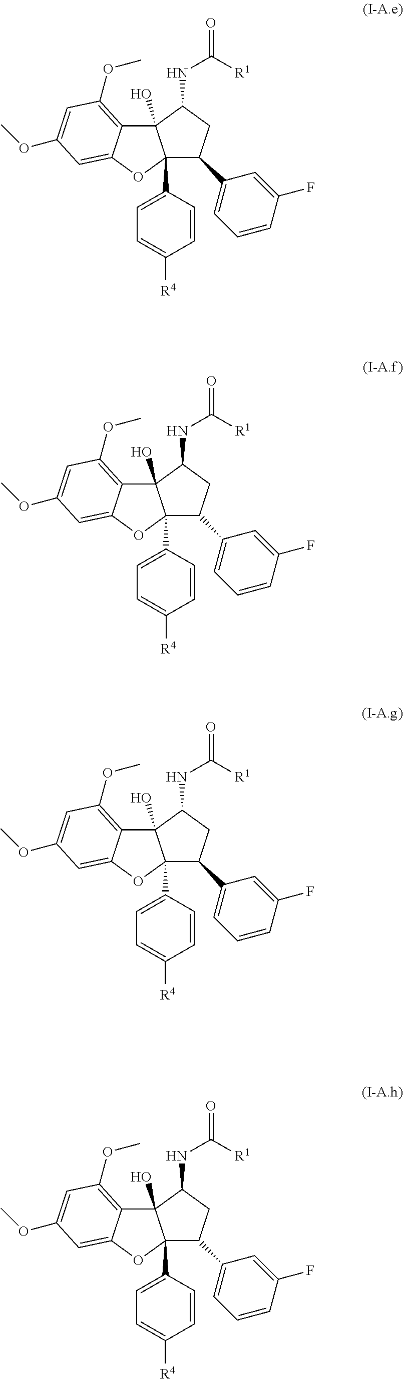

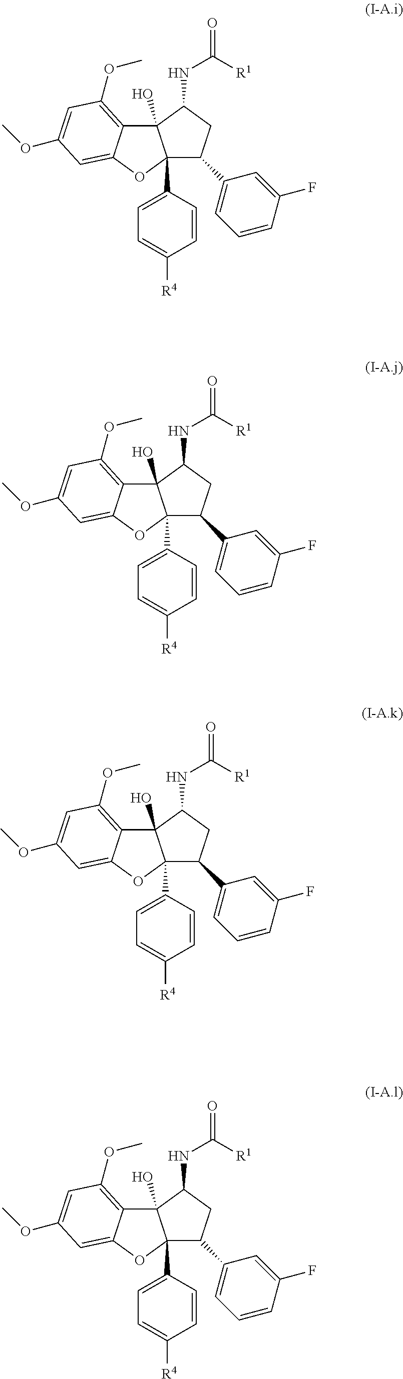

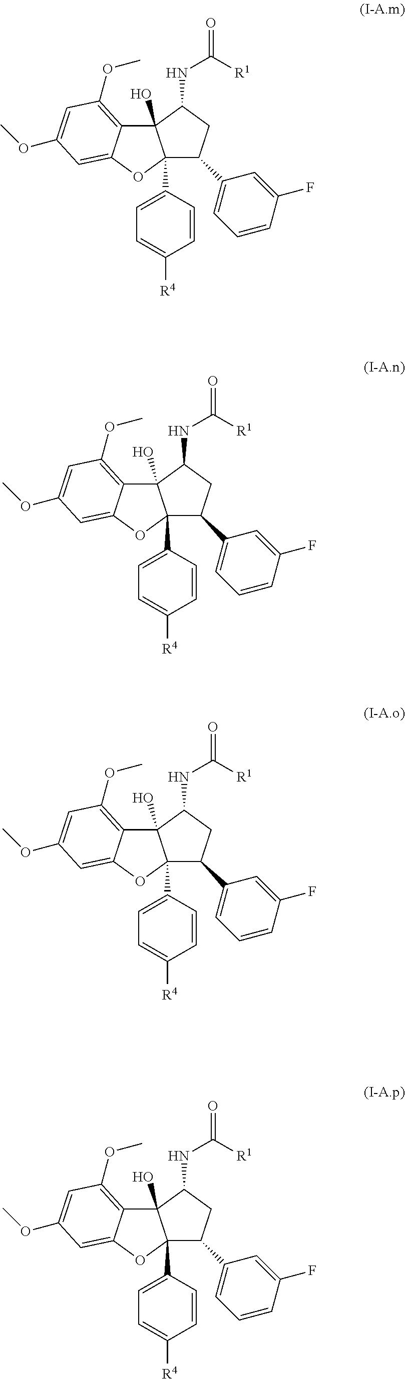

wherein, R¹ and R⁴ have one of the meanings as defined above or below.

The compounds of formulae (I-A.a) to (I-A.p) are specified by their absolute stereochemistry.

In a preferred embodiment the compound of formula (I) is a mixture of at least two enantiomers (I.a) to (I.p) or (I-A.a) to (I-A.p) or a mixture of the pharmaceutically acceptable salt thereof, wherein one enantiomer is enriched.

Preferably the compound of formula (I) is a mixture of (I.a) and (I.b) or a mixture of the pharmaceutically acceptable salt thereof, wherein the enantiomer excess (ee) of the enantiomer of formula (I.a) is at least 20%, preferably at least 50%, in particular at least 80%, especially at least 99%.

Preferably the compound of formula (I) is a mixture of (I-A.a) and (I-A.b) or a mixture of the pharmaceutically acceptable salt thereof, wherein the enantiomer excess (ee) of the enantiomer of formula (I-A.a) is at least 20%, preferably at least 50%, in particular at least 80%, especially at least 99%.

Preferred are compound of formula (I) according to the invention, or a pharmaceutically acceptable salt thereof, wherein R⁸ and R⁹ are both OCH₃.

Further, preferred are compound of formula (I) according to the invention, or a pharmaceutically acceptable salt thereof, wherein R⁶ is F and R⁷ is hydrogen.

Preferred are compounds of formula (I), wherein R¹ is selected from
C₁-C₄ alkyl, which is unsubstituted or substituted by 1, 2 or 3 substituents R^{a}; or
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom; or
NR²R³, wherein R² and R³ independently from each other are selected from hydrogen, C₁-C₄ alkyl and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c} S, SO and SO₂, wherein the heterocyclic ring is unsubstituted or substituted by 1, 2, 3, 4 or 5 identical or different radicals R^{d}; or
R² and R³ together with the nitrogen atom, which they are attached to, form a saturated or partly unsaturated bi- or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R^{f}; or
R² and R³ together with the nitrogen atom, which they are attached to, form a saturated or partly unsaturated spiro moiety, comprising 1, 2 identical or different heteroatoms or heteroatom-containing groups as ring members, selected from N, NR^{c} and O and wherein the heterocyclic ring is unsubstituted or substituted by 1, 2 or 3 identical or different radicals R^{g};
R⁴, R^{a}, R^{b}, R^{c}, R^{d}, R^{e}, R^{f} and R^{g} have one of the meanings as defined above and below.

Preferred are compounds of formula (I), wherein R¹ is selected from
C₁-C₄ alkyl, which is unsubstituted or substituted by 1, 2 or 3 substituents R^{a};
NR²R³, wherein R² and R³ independently from each other are selected from hydrogen and C₁-C₄ alkyl, and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c} or O, wherein the heterocyclic rings are unsubstituted or substituted by 1, 2, 3, 4 or 5 identical or different radicals R^{d}.

In particular R¹ is is selected from
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1 or 2 identical or different radicals R^{e} and wherein the heterocyloalkyl is connected to the remaining molecule via a carbon atom; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system; or
R² and R³ together with the nitrogen atom, which they are attached to, form a saturated or partly unsaturated spiro moiety, comprising 2 identical or different heteroatoms or heteroatom-containing groups as ring members, selected from N, NR^{c} and O, preferably R² and R³ together with the nitrogen atom, which they are attached to, form a 2-oxa-6-azaspiro[3.3]heptan-6-yl spiro compound.

More preferably, R¹ is is selected from
C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom, preferably C₅-C₇ heterocyloalkyl is selected from pyrrolidinyl and piperidinyl; or
NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents preferably hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c}, SO₂ and O, wherein R^{c} is selected from hydrogen, C₁-C₄-alkyl, wherein the heterocyclic ring is unsubstituted or substituted by 1 or 2 identical or different radicals selected from C₁-C₄-alkyl, C₁-C₄-hydroxyalkyl, C₁-C₄-alkoxy, NH₂, N(C₁-C₂ alkyl)₂ and NH(C₁-C₂ alkyl), preferably R² and R³ together with the nitrogen atom, which they are attached to, form a pyrrolidine ring, piperazine ring, acetidine ring or morpholine ring wherein the pyrrolidine ring, piperazine ring, acetidine ring, or morpholine ring is unsubstituted or substituted by 1 or 2 substituents selected from C₁-C₄-alkyl, C₁-C₄-hydroxyalkyl, C₁-C₄-alkoxy, NH₂, N(C₁-C₂ alkyl)₂, NH(C₁-C₂ alkyl) carbonyloxy-C₁-C₂-alkyl; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system.

Particularly, R¹ is NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents, especially R² and R³ independently from each other are selected from hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O, wherein R^{c} is selected from hydrogen and C₁-C₄-alkyl. Preferably R² and R³ together with the nitrogen atom, which they are attached to, form a pyrrolidine ring, piperazine ring, acetidin ring or morpholin ring, wherein the pyrrolidine ring, piperazine ring, acetidin ring and morpholin ring is unsubstituted or substituted by 1 or 2 substituents selected from C₁-C₄-alkyl, C₁-C₄-hydroxyalkyl and C₁-C₄-alkoxy.

In particular, R¹ is selected from methyl, 4-methyl-piperazin-1-yl, pyrrolidin-1-yl, N,N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino, (cyclopropylmethyl)amino, 8-methyl-3,8-diazabicyclo[3.2.1]octan-3-yl, 1-methylpiperidin-4-yl, thiomorpholine-4-yl-1,1dioxide (1λ⁶-thiomorpholine-1,1-dionyl), 3-(dimethylamino)azetidin-1-yl, 4-(dimethylamino)piperidin-1-yl, N-ethan-1-ol-amino, azetidine-3-carbonyloxymethyl, N,N-dimethylamino methyl, 2-oxa-6-azaspiro[3.3]heptan-6-yl and pyrrolidine-3-yl especially, methyl, 4-methyl-piperazin-1-yl, pyrrolidin-1-yl, N,N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino, (cyclopropylmethyl)amino.

In a first preferred embodiment R¹ is selected from C₁-C₄ alkyl, which is unsubstituted or substituted by 1, 2 or 3 substituents R^{a}, in particular R¹ is selected from C₁-C₂ alkyl, especially R¹ is methyl.

In a second preferred embodiment R¹ is selected from NR²R³, wherein R² and R³ independently from each other are selected from hydrogen and C₂-C₃ alkyl, which is unsubstituted or substituted by 1 or 2 substituents R^{a}, preferably C₂-C₃ alkyl, which is unsubstituted. In particular, R¹ is hydrogen and R² is C₂-C₃ alkyl, which is unsubstituted, especially, R¹ is hydrogen and R² is selected from ethyl and isopropyl.

In a third preferred embodiment R¹ is selected from NR²R³, wherein R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O. Preferably R² and R³ together with the nitrogen atom, which they are attached to, form a pyrrolidine ring, piperazine ring, acetidin ring or morpholin ring, wherein the pyrrolidine ring, piperazine ring, acetidin ring or morpholin ring is unsubstituted or substituted by 1 or 2 substituents selected from C₁-C₄-alkyl, C₁-C₄-hydroxyalkyl and C₁-C₄-alkoxy.

In particular R² and R³ together with the nitrogen atom, which they are attached to, form a piperazin-1-yl ring, N-, acetidin-1-yl or morpholin-4-yl or pyrrolidin-1-yl ring, especially a 4-methyl-piperazin-1-yl ring, pyrrolidin-1-yl ring, acetidin-1-yl or morpholin-4-yl.

In a fourth preferred embodiment R¹ is selected from C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom, in particular R¹ is selected from C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom.

In a fifth preferred embodiment R¹ is selected from NR²R³, wherein R² and R³ together with the nitrogen atom, which they are attached to, form a saturated or partly unsaturated bi- or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R^{f}, in particular R¹ is selected from NR²R³, wherein R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system.

In a sixth preferred embodiment R¹ is selected from NR²R³, wherein R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g}, in particular R¹ is selected from NR²R³, wherein R² and R³ together with the nitrogen atom, which they are attached to, form a saturated or partly unsaturated spiro compound, comprising 1 or 2 identical or different heteroatoms or heteroatom-containing groups as ring members, selected from N, NR^{c} and O and wherein the heterocyclic ring is unsubstituted or substituted by 1, 2 or 3 identical or different radicals R^{g}, preferably R² and R³ together with the nitrogen atom, which they are attached to, form saturated or partly unsaturated spiro moiety, comprising 2 identical or different heteroatoms or heteroatom-containing groups as ring members, selected from N, NR^{c} and O, especially R² and R³ together with the nitrogen atom, which they are attached to, form a 2-oxa-6-azaspiro[3.3]heptan-6-yl spiro compound. In particular R⁴ is selected from Cl, CN and C₃-C₆ cycloalkyl. Especially, R⁴ is selected from Cl, CN and cyclopropyl.

Irrespective of its occurrence, R^{a} is preferably selected from C₁-C₂-alkyl, C₃-C₆ cycloalkyl, and phenyl, wherein C₃-C₆ cycloalkyl and phenyl is unsubstituted or substituted by 1 or 2 substituents selected from F and Cl. Especially, R^{a} is selected from C₁-C₂-alkyl, C₃-C₆ cycloalkyl, and 4-fluoro-phenyl.

Irrespective of its occurrence, R^{b} is preferably selected from C₁-C₂-alkyl.

Irrespective of its occurrence, R^{c} is preferably selected from hydrogen and C₁-C₄-alkyl, in particular C₁-C₂-alkyl, especially methyl.

Irrespective of its occurrence, R^{d} is preferably selected from C₁-C₄-alkyl, C₁-C₄-hydroxyalkyl and C₁-C₄-alkoxy, in particular C₁-C₂-alkyl and C₁-C₂-hydroxyalkyl.

Irrespective of its occurrence, R^{e} is selected from halogen, C₁-C₄-alkyl, C₁-C₄-haloalkyl and NR^{5a}R^{5b}.

Irrespective of its occurrence, R^{f} is preferably selected from halogen, C₁-C₄-alkyl and C₁-C₄-haloalkyl.

Irrespective of its occurrence, R^{g} is preferably selected from halogen, C₁-C₄-alky and C₁-C₄-haloalkyl.

Irrespective of its occurrence, R^{h} is preferably selected from halogen, C₁-C₄-alkyl and C₁-C₄-haloalkyl.

Irrespective of its occurrence, R^{5a} and R^{5b} independently of each other are preferably selected from hydrogen and C₁-C₄-alkyl.

Another preferred embodiment are the compound of formula (I) as defined above and below or a mixture of the pharmaceutically acceptable salt thereof, which is a racemic mixture (I.a′) or (I-A.a′)

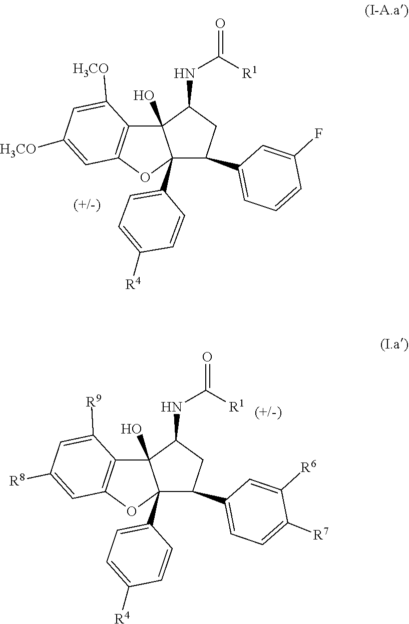

wherein R¹, R⁴, R⁶, R⁷, R⁸ and R⁹ have the meanings as defined above.

Another preferred embodiment is the compound of formula (I), which is a compound of formula (I.a) or an enantiomeric mixture comprising the compounds of formula (I.a) and (I.b) or the pharmaceutically acceptable salt thereof,

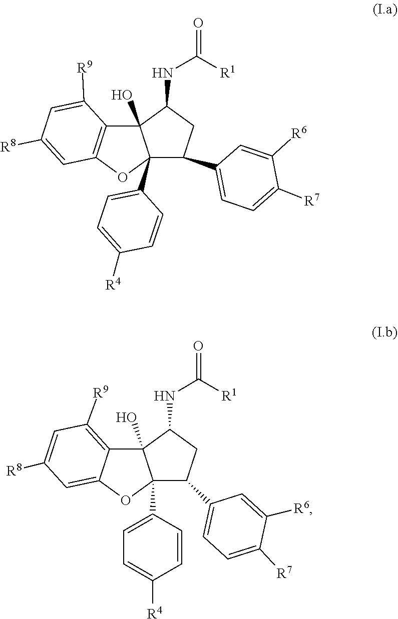

wherein R¹, R⁴, R⁶, R⁷, R⁸ and R⁹ have the same meanings as defined above and below.

In particular, the compound of formula (I) is a mixture of (I.a) and (I.b) or a mixture of the pharmaceutically acceptable salt thereof, wherein the enantiomeric excess (ee) of the enantiomer of formula (I.a) is at least 20%, preferably at least 50%, in particular at least 80%, especially at least 99%.

Especially the compound of formula (I) is a compound (I.a) or (I-A.a) or a pharmaceutically acceptable salt thereof,

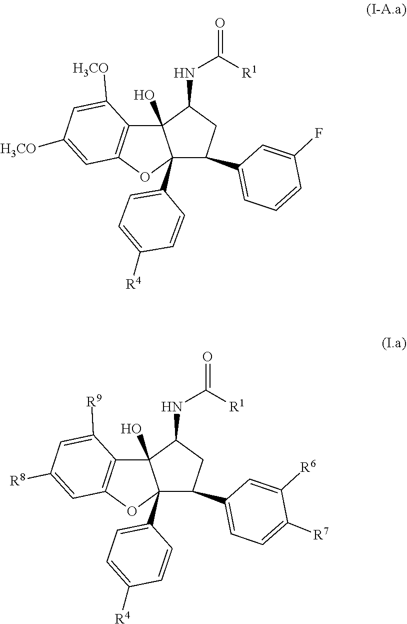

wherein R¹, R⁴, R⁶, R⁷, R⁸ and R⁹ have the meanings as defined above.

Preferred are compounds of formulae (I.a), or the racemic mixture (I.a′), or an enantiomeric mixture comprising the compounds of formula (I.a) and (I.b) in a different ratios from 1:1, or a pharmaceutically acceptable salt thereof, wherein R¹ is selected from
C₁-C₄ alkyl, wherein alkyl is unsubstituted or substituted by 1, 2 or 3 substituents R^{a}; or
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom; or
NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d}; or
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f}; or
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g}; or
R^{a} is selected from halogen, OH, C₃-C₇ cycloalkyl, C₁-C₃ alkoxy, phenyl, NR^{5a}R^{5b}, C₁-C₄-alkylsulfonyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b};
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃ and OCD₃;
R⁹ is selected from OCH₃ and OCD₃.

Preferred are compounds of formula (I) according to the invention, or a pharmaceutically acceptable salt thereof, wherein R⁸ and R⁹ are both OCH₃.

Further, preferred are compounds of formula (I) according to the invention, or a pharmaceutically acceptable salt thereof, wherein R⁶ is F and R⁷ is hydrogen.

Preferred are compounds of formulae (I.a), (I.a′) or a pharmaceutically acceptable salt thereof, wherein R¹ is selected from
C₁-C₄ alkyl, which is unsubstituted or substituted by 1, 2 or 3 substituents R^{a};
NR²R³, wherein R² and R³ independently from each other are selected from hydrogen and C₁-C₄ alkyl and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c} and O, wherein the heterocyclic rings are unsubstituted or substituted by 1, 2, 3, 4 or 5 identical or different radicals R^{d}.

In particular R¹ is is selected from
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1 or 2 identical or different radicals R^{e} and wherein the heterocyloalkyl is connected to the remaining molecule via a carbon atom; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system; or
R² and R³ together with the nitrogen atom, which they are attached to, form a saturated or partly unsaturated spiro moiety, comprising 2 identical or different heteroatoms or heteroatom-containing groups as ring members, selected from N, NR^{c} and O, preferably R² and R³ together with the nitrogen atom, which they are attached to, form a 2-oxa-6-azaspiro[3.3]heptan-6-yl spiro compound.

More preferably, R¹ is is selected from
C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom, preferably C₅-C₇ heterocyloalkyl is selected from pyrrolidinyl and piperidinyl; or
NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents, preferably hydrogen, C₂-C₃ alkyl, which is unsubstituted, and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c}, SO₂ and O, wherein R^{c} is selected from hydrogen, C₁-C₄-alkyl, wherein the heterocyclic ring is unsubstituted or substituted by 1 or 2 identical or different radicals selected from C₁-C₄-alkyl, C₁-C₄-hydroxyalkyl, C₁-C₄-alkoxy, NH₂, N(C₁-C₂ alkyl)₂ and NH(C₁-C₂ alkyl), preferably R² and R³ together with the nitrogen atom, which they are attached to, form a pyrrolidine ring, piperazine ring, acetidin ring or morpholin ring wherein the pyrrolidine ring, piperazine ring, acetidin ring or morpholin ring is unsubstituted or substituted by 1 or 2 substituents selected from C₁-C₄-alkyl, C₁-C₄-hydroxyalkyl, C₁-C₄-alkoxy, NH₂, N(C₁-C₂ alkyl)₂, NH(C₁-C₂ alkyl) carbonyloxy-C₁-C₂-alkyl; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system.

Particularly, R¹ is NR²R³, wherein R² and R³ independently from each other are selected from hydrogen and C₁-C₃ alkyl, and C₃-C₆ cycloalkyl, wherein alkyl is unsubstituted or substituted by 1 or 2 substituents R^{a}, wherein cycloalkyl is unsubstituted or substituted by 1 or 2 substituents R^{b}, especially R² and R³ independently from each other are selected from hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O, wherein R^{c} is selected from hydrogen and C₁-C₄-alkyl. Preferably R² and R³ together with the nitrogen atom, which they are attached to, form a pyrrolidine ring, piperazine ring, acetidin ring or morpholin ring, wherein the pyrrolidine ring, piperazine ring, acetidin ring and morpholin ring is unsubstituted or substituted by 1 or 2 substituents selected from C₁-C₄-alkyl, C₁-C₄-hydroxyalkyl and C₁-C₄-alkoxy.

In particular, R¹ is selected from methyl, 4-methyl-piperazin-1-yl, pyrrolidin-1-yl, N,N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino, (cyclopropylmethyl)amino, 8-methyl-3,8-diazabicyclo[3.2.1]octan-3-yl, 1-methylpiperidin-4-yl, thiomorpholine-4-yl-1,1dioxide (1λ⁶-thiomorpholine-1,1-dionyl), 3-(dimethylamino)azetidin-1-yl, 4-(dimethylamino)piperidin-1-yl, N-ethan-1-ol-amino, azetidine-3-carbonyloxymethyl, N.N-dimethylaminometkyl, 2-oxa-6-azaspiro[3.3]heptan-6-yl and pyrrolidine-3-yl especially, methyl, 4-methyl-piperazin-1-yl, pyrrolidin-1-yl, N,N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino and (cyclopropylmethyl)amino.

In a first preferred embodiment R¹ is selected from C₁-C₄ alkyl, which is unsubstituted or substituted by 1, 2 or 3 substituents R^{a}, in particular R¹ is selected from C₁-C₂ alkyl, especially R¹ is methyl.

In a second preferred embodiment R¹ is selected from NR²R³, wherein R² and R³ independently from each other are selected from hydrogen and C₂-C₃ alkyl, which is unsubstituted or substituted by 1 or 2 substituents R^{a}, preferably C₂-C₃ alkyl, which is unsubstituted. In particular, R¹ is hydrogen and R² is C₂-C₃ alkyl, which is unsubstituted, especially, R¹ is hydrogen and R² is selected from ethyl and isopropyl.

In a third preferred embodiment R¹ is selected from NR²R³, wherein R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c} and O. Preferably R² and R³ together with the nitrogen atom, which they are attached to, form a pyrrolidine ring, piperazine ring, acetidin ring or morpholin ring, wherein the pyrrolidine ring, piperazine ring, acetidin ring or morpholin ring is unsubstituted or substituted by 1 or 2 substituents selected from C₁-C₄-alkyl, C₁-C₄-hydroxyalkyl and C₁-C₄-alkoxy. In particular R² and R³ together with the nitrogen atom, which they are attached to, form a piperazin-1-yl ring, N-acetidin-1-yl ring or morpholin-4-yl ring or pyrrolidin-1-yl ring, especially a 4-methyl-piperazin-1-yl ring, pyrrolidin-1-yl ring, acetidin-1-yl or morpholin-4-yl.

In a fourth preferred embodiment R¹ is selected from C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom, in particular R¹ is selected from C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom.

In a fifth preferred embodiment R¹ is selected NR²R³, wherein from R² and R³ together with the nitrogen atom, which they are attached to, form a saturated or partly unsaturated bi- or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R′, in particular R¹ is selected NR²R³, wherein from R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}. Preferably R² and R³ together with the nitrogen atom, which they are attached to, form a 8-methyl-3,8-diazabicyclo[3.2.1]octan-3-yl ring system.

In a sixth preferred embodiment R¹ is selected from NR²R³, wherein R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R⁹, in particular R¹ is selected from NR²R³, wherein R² and R³ together with the nitrogen atom, which they are attached to, form a saturated or partly unsaturated spiro moiety, comprising 1, 2 identical or different heteroatoms or heteroatom-containing groups as ring members, selected from N, NR^{c} and O and wherein the heterocyclic ring is unsubstituted or substituted by 1, 2 or 3 identical or different radicals R^{g}, preferably R² and R³ together with the nitrogen atom, which they are attached to, form saturated or partly unsaturated spiro compound, comprising 2 identical or different heteroatoms or heteroatom-containing groups as ring members, selected from N, NR^{c} and O, especially R² and R³ together with the nitrogen atom, which they are attached to, form a 2-oxa-6-azaspiro[3.3]heptan-6-yl spiro compound.

In particular R⁴ is selected from Cl, CN and C₃-C₆ cycloalkyl. Especially, R⁴ is selected from Cl, CN and cyclopropyl.

Another special embodiment are the compounds of formula (I-A)

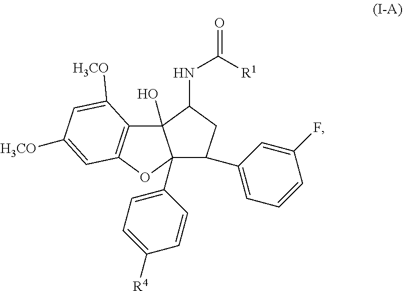

wherein R¹ and R⁴ are selected from the definition given in one line of table 1:

TABLE 1

| Nr. | R¹ | R⁴ |
|---|---|---|
| 1. | 4-methyl-piperazin-1-yl | Cl |
| 2. | pyrrolidin-1-yl | Cl |
| 3. | N,N-diethylamino | Cl |
| 4. | N-isopropylamino | Cl |
| 5. | N-ethylamino | Cl |
| 6. | N,N-methyl-isopropylamino | Cl |
| 7. | acetidin-1-yl | Cl |
| 8. | morpholin-4-yl | Cl |
| 9. | N-cyclopentylamino | Cl |
| 10. | [1-(4-fluorophenyl)ethyl]amino | Cl |
| 11. | (cyclopropylmethyl)amino | Cl |
| 12. | methyl | Cl |
| 13. | 8-methyl-3,8- | Cl |
| | diazabicyclo[3.2.1]octan-3-yl | |
| 14. | 1-methylpiperidin-4-yl | Cl |
| 15. | thiomorpholine-4-yl-1,1dioxide | Cl |
| 16. | 3-(dimethylamino)azetidin-1-yl | Cl |
| 17. | 4-(dimethylamino)piperidin-1-yl | Cl |
| 18. | N-ethan-1-ol-amino | Cl |
| 19. | azetidine-3-carbonyloxy methyl | Cl |
| 20. | N,N-dimethylamino methyl | Cl |
| 21. | 2-oxa-6-azaspiro[3.3]heptan-6-yl | Cl |
| 22. | pyrrolidine-3-yl | Cl |
| 23. | (piperazin-1-yl)ethyl | Cl |
| 24. | 4-methyl-piperazin-1-yl | cyclopropyl |
| 25. | pyrrolidin-1-yl | cyclopropyl |
| 26. | N,N-diethylamino | cyclopropyl |
| 27. | N-isopropylamino | cyclopropyl |
| 28. | N-ethylamino | cyclopropyl |
| 29. | N,N-methyl-isopropylamino | cyclopropyl |
| 30. | acetidin-1-yl | cyclopropyl |
| 31. | morpholin-4-yl | cyclopropyl |
| 32. | N-cyclopentylamino | cyclopropyl |
| 33. | [1-(4-fluorophenyl)ethyl]amino | cyclopropyl |
| 34. | (cyclopropylmethyl)amino | cyclopropyl |
| 35. | methyl | cyclopropyl |
| 36. | 8-methyl-3,8- | cyclopropyl |
| | diazabicyclo[3.2.1]octan-3-yl | |
| 37. | 1-methylpiperidin-4-yl | cyclopropyl |
| 38. | thiomorpholine-4-yl-1,1dioxide | cyclopropyl |
| 39. | 3-(dimethylamino)azetidin-1-yl | cyclopropyl |
| 40. | 4-(dimethylamino)piperidin-1-yl | cyclopropyl |
| 41. | N-ethan-1-ol-amino | cyclopropyl |
| 42. | azetidine-3-carbonyloxy methyl | cyclopropyl |
| 43. | N,N-dimethylamino methyl | cyclopropyl |
| 44. | 2-oxa-6-azaspiro[3.3]heptan-6-yl | cyclopropyl |
| 45. | pyrrolidine-3-yl | cyclopropyl |
| 46. | (piperazin-1-yl)ethyl | cyclopropyl |
| 47. | 4-methyl-piperazin-1-yl | CN |
| 48. | pyrrolidin-1-yl | CN |
| 49. | N,N-diethylamino | CN |
| 50. | N-isopropylamino | CN |
| 51. | N-ethylamino | CN |
| 52. | N,N-methyl-isopropylamino | CN |
| 53. | acetidin-1-yl | CN |
| 54. | morpholin-4-yl | CN |
| 55. | N-cyclopentylamino | CN |
| 56. | [1-(4-fluorophenyl)ethyl]amino | CN |
| 57. | (cyclopropylmethyl)amino | CN |
| 58. | methyl | CN |
| 59. | 8-methyl-3,8- | CN |
| | diazabicyclo[3.2.1]octan-3-yl | |
| 60. | 1-methylpiperidin-4-yl | CN |
| 61. | thiomorpholine-4-yl-1,1dioxide | CN |
| 62. | 3-(dimethylamino)azetidin-1-yl | CN |
| 63. | 4-(dimethylamino)piperidin-1-yl | CN |
| 64. | N-ethan-1-ol-amino | CN |
| 65. | azetidine-3-carbonyloxy methyl | CN |
| 66. | N,N-dimethylamino methyl | CN |
| 67. | 2-oxa-6-azaspiro[3.3]heptan-6-yl | CN |
| 68. | pyrrolidine-3-yl | CN |
| 69. | (piperazin-1-yl)ethyl | CN |

Another special embodiment are the compounds of formula (I-A.a′)

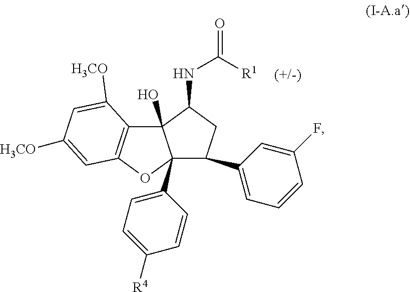

wherein R¹ and R⁴ have one of the meanings selected from the definition given in one line of table 1 above.

Another special embodiment are the compounds of formula (I-A.a)

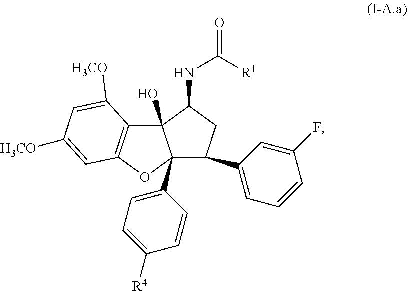

wherein R¹ and R⁴ have one of the meanings selected from the definition given in one line of table 1 above.

Another special embodiment are the compounds of formula of formula (I), an enantiomeric mixture comprising the compounds of formula (I-A.a) and (I-A.b) or the pharmaceutically acceptable salt thereof,

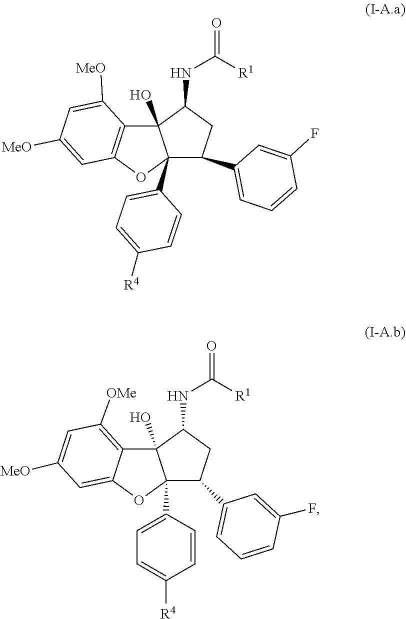

wherein R¹ and R⁴ have one of the meanings selected from the definition given in one line of table 1 above.

In particular compound of formula (I) is a mixture of (I-A.a) and (I-A.b), wherein the enantiomer excess (ee) of the enantiomer of formula (I-A.a) is at least 20%, preferably at least 50%, in particular at least 80%, especially at least 99% and wherein R¹ and R⁴ have one of the meanings selected from the definition given in one line of table 1 above.

Another special embodiment are the compounds of formula (I.b)

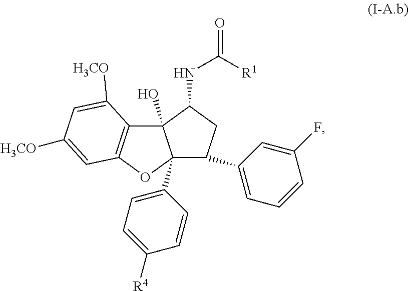

wherein R¹ and R⁴ have one of the meanings selected from the definition given in one line of table 1 above.

Another special embodiment are the compounds selected from A, B, C, D, E, F, G, H, I, J, K, L, M, N, O, P, Q, R, S, T, U, V and the mixture of the each of compounds A to V with its respective enantiomer:

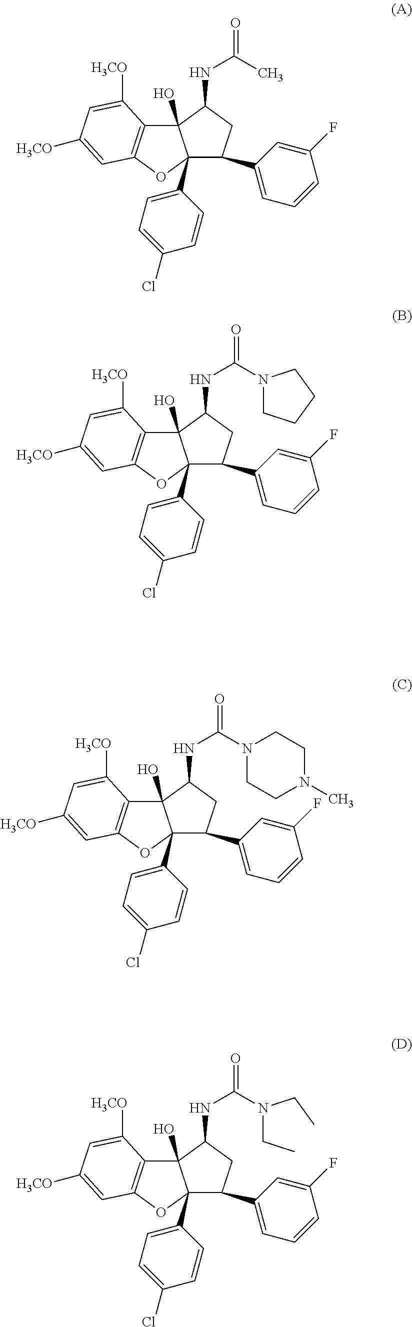 [SMILES: COc1cc(OC)c2c(c1)O[C@@]1(c3ccc(Cl)cc3)[C@H](c3cccc(F)c3)C[C@H](NC(C)=O)[C@@]21O ; COc1cc(OC)c2c(c1)O[C@@]1(c3ccc(Cl)cc3)[C@H](c3cccc(F)c3)C[C@H](NC(=O)N3CCCC3)[C@@]21O ; COc1cc(OC)c2c(c1)O[C@@]1(c3ccc(Cl)cc3)[C@H](c3cccc(F)c3)C[C@H](NC(=O)N3CCN(C)CC3)[C@@]21O ; CCN(CC)C(=O)N[C@H]1C[C@@H](c2cccc(F)c2)[C@]2(c3ccc(Cl)cc3)Oc3cc(OC)cc(OC)c3[C@]12O]

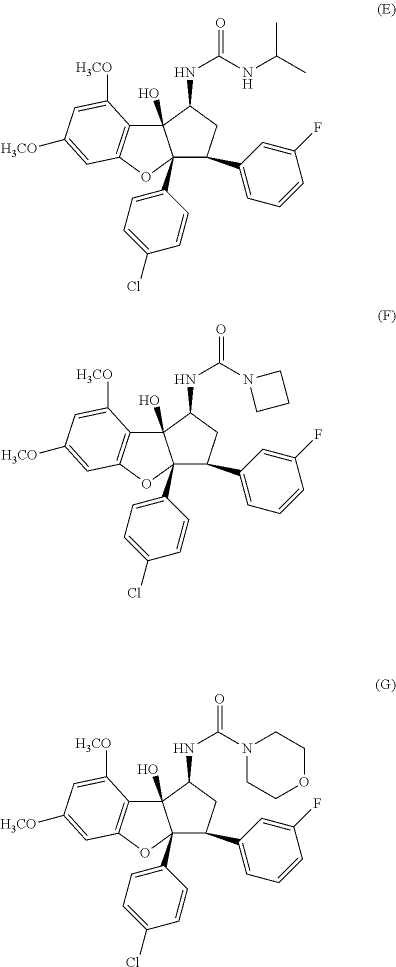 [SMILES: COc1cc(OC)c2c(c1)O[C@@]1(c3ccc(Cl)cc3)[C@H](c3cccc(F)c3)C[C@H](NC(=O)NC(C)C)[C@@]21O ; COc1cc(OC)c2c(c1)O[C@@]1(c3ccc(Cl)cc3)[C@H](c3cccc(F)c3)C[C@H](NC(=O)N3CCC3)[C@@]21O ; COc1cc(OC)c2c(c1)O[C@@]1(c3ccc(Cl)cc3)[C@H](c3cccc(F)c3)C[C@H](NC(=O)N3CCOCC3)[C@@]21O]

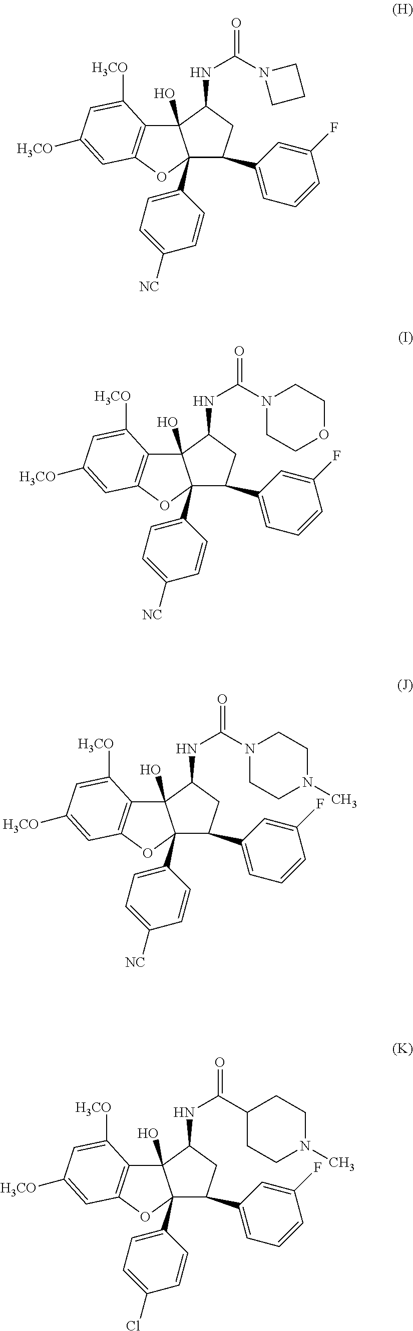 [SMILES: COc1cc(OC)c2c(c1)O[C@@]1(c3ccc(C#N)cc3)[C@H](c3cccc(F)c3)C[C@H](NC(=O)N3CCC3)[C@@]21O ; COc1cc(OC)c2c(c1)O[C@@]1(c3ccc(C#N)cc3)[C@H](c3cccc(F)c3)C[C@H](NC(=O)N3CCOCC3)[C@@]21O ; COc1cc(OC)c2c(c1)O[C@@]1(c3ccc(C#N)cc3)[C@H](c3cccc(F)c3)C[C@H](NC(=O)N3CCN(C)CC3)[C@@]21O ; COc1cc(OC)c2c(c1)O[C@@]1(c3ccc(Cl)cc3)[C@H](c3cccc(F)c3)C[C@H](NC(=O)C3CCN(C)CC3)[C@@]21O]

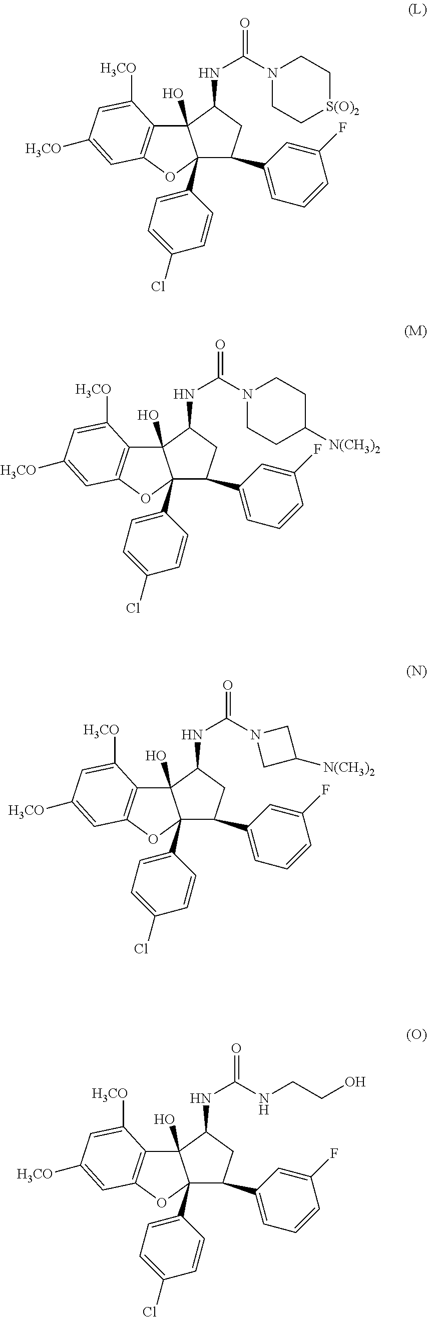

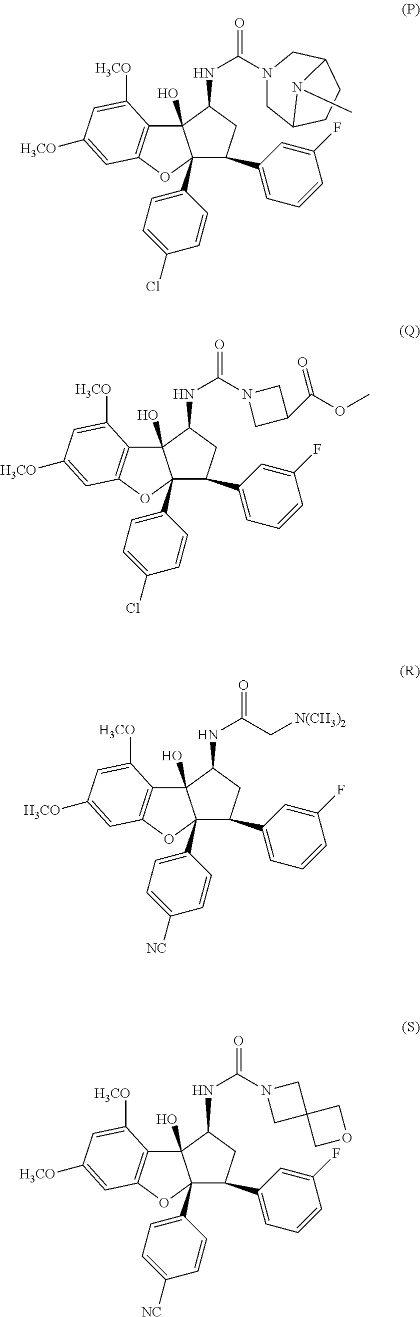 [SMILES: COc1cc(OC)c2c(c1)O[C@@]1(c3ccc(Cl)cc3)[C@H](c3cccc(F)c3)C[C@H](NC(=O)N3CC4CCC(C3)N4C)[C@@]21O ; COC(=O)C1CN(C(=O)N[C@H]2C[C@@H](c3cccc(F)c3)[C@]3(c4ccc(Cl)cc4)Oc4cc(OC)cc(OC)c4[C@]23O)C1 ; COc1cc(OC)c2c(c1)O[C@@]1(c3ccc(C#N)cc3)[C@H](c3cccc(F)c3)C[C@H](NC(=O)CN(C)C)[C@@]21O ; COc1cc(OC)c2c(c1)O[C@@]1(c3ccc(C#N)cc3)[C@H](c3cccc(F)c3)C[C@H](NC(=O)N3CC4(COC4)C3)[C@@]21O]

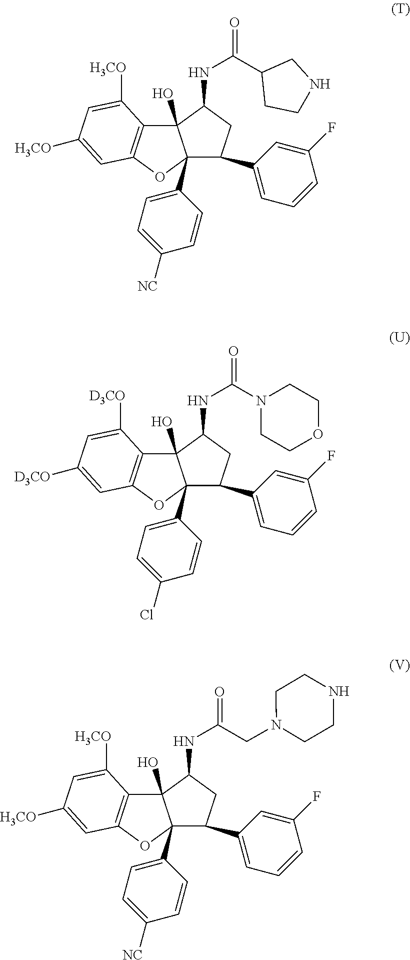

or mixtures thereof.

Preferred is a compound of formula (A) or an enantiomeric mixture comprising the compounds of formula (A) and its enantiomer, in particular wherein the enantiomer excess (ee) of the enantiomer of formula (A) is at least 20%, preferably at least 50%, in particular at least 80%, especially at least 99%.

Preferred is a compound of formula (B) or an enantiomeric mixture comprising the compounds of formula (B) and its enantiomer, in particular wherein the enantiomer excess (ee) of the enantiomer of formula (B) is at least 20%, preferably at least 50%, in particular at least 80%, especially at least 99%.

Preferred is a compound of formula (C) or an enantiomeric mixture comprising the compounds of formula (C) and its enantiomer, in particular wherein the enantiomer excess (ee) of the enantiomer of formula (C) is at least 20%, preferably at least 50%, in particular at least 80%, especially at least 99%.

Preferred is a compound of formula (D) or an enantiomeric mixture comprising the compounds of formula (D) and its enantiomer, in particular wherein the enantiomer excess (ee) of the enantiomer of formula (D) is at least 20%, preferably at least 50%, in particular at least 80%, especially at least 99%.

Preferred is a compound of formula (E) or an enantiomeric mixture comprising the compounds of formula (E) and its enantiomer, in particular wherein the enantiomer excess (ee) of the enantiomer of formula (E) is at least 20%, preferably at least 50%, in particular at least 80%, especially at least 99%.

Preferred is a compound of formula (F) or an enantiomeric mixture comprising the compounds of formula (F) and its enantiomer, in particular wherein the enantiomer excess (ee) of the enantiomer of formula (F) is at least 20%, preferably at least 50%, in particular at least 80%, especially at least 99%.

Preferred is a compound of formula (G) or an enantiomeric mixture comprising the compounds of formula (G) and its enantiomer, in particular wherein the enantiomer excess (ee) of the enantiomer of formula (G) is at least 20%, preferably at least 50%, in particular at least 80%, especially at least 99%.

Preferred is a compound of formula (H) or an enantiomeric mixture comprising the compounds of formula (H) and its enantiomer, in particular wherein the enantiomer excess (ee) of the enantiomer of formula (H) is at least 20%, preferably at least 50%, in particular at least 80%, especially at least 99%.

Preferred is a compound of formula (I) or an enantiomeric mixture comprising the compounds of formula (I) and its enantiomer, in particular wherein the enantiomer excess (ee) of the enantiomer of formula (I) is at least 20%, preferably at least 50%, in particular at least 80%, especially at least 99%.

Preferred is a compound of formula (J) or an enantiomeric mixture comprising the compounds of formula (J) and its enantiomer, in particular wherein the enantiomer excess (ee) of the enantiomer of formula (J) is at least 20%, preferably at least 50%, in particular at least 80%, especially at least 99%.

Preferred is a compound of formula (K) or an enantiomeric mixture comprising the compounds of formula (K) and its enantiomer, in particular wherein the enantiomer excess (ee) of the enantiomer of formula (K) is at least 20%, preferably at least 50%, in particular at least 80%, especially at least 99%.

Preferred is a compound of formula (L) or an enantiomeric mixture comprising the compounds of formula (L) and its enantiomer, in particular wherein the enantiomer excess (ee) of the enantiomer of formula (L) is at least 20%, preferably at least 50%, in particular at least 80%, especially at least 99%.

Preferred is a compound of formula (M) or an enantiomeric mixture comprising the compounds of formula (M) and its enantiomer, in particular wherein the enantiomer excess (ee) of the enantiomer of formula (M) is at least 20%, preferably at least 50%, in particular at least 80%, especially at least 99%.

Preferred is a compound of formula (N) or an enantiomeric mixture comprising the compounds of formula (N) and its enantiomer, in particular wherein the enantiomer excess (ee) of the enantiomer of formula (N) is at least 20%, preferably at least 50%, in particular at least 80%, especially at least 99%.

Preferred is a compound of formula (O) or an enantiomeric mixture comprising the compounds of formula (O) and its enantiomer, in particular wherein the enantiomer excess (ee) of the enantiomer of formula (O) is at least 20%, preferably at least 50%, in particular at least 80%, especially at least 99%.

Preferred is a compound of formula (Q) or an enantiomeric mixture comprising the compounds of formula (Q) and its enantiomer, in particular wherein the enantiomer excess (ee) of the enantiomer of formula (Q) is at least 20%, preferably at least 50%, in particular at least 80%, especially at least 99%.

Preferred is a compound of formula (R) or an enantiomeric mixture comprising the compounds of formula (R) and its enantiomer, in particular wherein the enantiomer excess (ee) of the enantiomer of formula (R) is at least 20%, preferably at least 50%, in particular at least 80%, especially at least 99%.

Preferred is a compound of formula (S) or an enantiomeric mixture comprising the compounds of formula (S) and its enantiomer, in particular wherein the enantiomer excess (ee) of the enantiomer of formula (S) is at least 20%, preferably at least 50%, in particular at least 80%, especially at least 99%.

Preferred is a compound of formula (T) or an enantiomeric mixture comprising the compounds of formula (T) and its enantiomer, in particular wherein the enantiomer excess (ee) of the enantiomer of formula (T) is at least 20%, preferably at least 50%, in particular at least 80%, especially at least 99%.

Preferred is a compound of formula (U) or an enantiomeric mixture comprising the compounds of formula (U) and its enantiomer, in particular wherein the enantiomer excess (ee) of the enantiomer of formula (U) is at least 20%, preferably at least 50%, in particular at least 80%, especially at least 99%.

Preferred at least a compound of formula (V) or an enantiomeric mixture comprising the compounds of formula (V) and its enantiomer, in particular wherein the enantiomer excess (ee) of the enantiomer of formula (V) is at least 20%, preferably at least 50%, in particular at least 80%, especially at least 99%.

Especially preferred is compound C

Especially preferred is compound I.

### Preparation

The compounds of the present invention can be synthesized using the methods described below, together with synthetic methods known in the art of synthetic organic chemistry, or variations thereof as appreciated by those skilled in the art. Preferred methods include, but are not limited to, those described below. All references cited herein are hereby incorporated by reference in their entirety.

The compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents that are compatible with the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene's Protective Groups In Organic Synthesis, Editor P. G. M. Wuts, Fifth Edition, Wiley and Sons 2014.

The invention relates to a method for the production of compounds of formula (I) comprising the steps a1) to a9) as defined above and in the following.

### Step a1)

In step a1) of the process according to the invention a compound of formula (II) is provided. Compounds (II) can be synthesized by standard methods known to a person skilled in the art. For example, the synthesis of 2-(4-chlorophenyl)-4,6-dimethoxybenzofuran-3(2H)-one, i.e. a compound (II) wherein R⁴ is Cl (denoted in the following also as SM4), is outlined in the following scheme 1 and described in reaction steps 1 to 4 of the working examples.

### Step a2)

Reaction of compound (II) with compound (III) in the sense of a Michael addition yields the adduct (IV). This reaction usually leads predominantly to a single diastereoisomer. A suitable base for the Michael addition is benzyltrimethylammonium hydroxide (Triton B).

### Step a3)

In reaction step a3), the aldehyde group of compound (IV) is subjected to a cyanosilylation with trimethylsilylcyanide (TMSCN) to give the cyanhydrin silylether (V). A preferred catalyst for the addition of TMSCN is zinc iodide. The ratio of the stereoisomers with regard to the two asymmetric carbon atoms carrying the two benzene rings remains unchanged.

### Step a4)

In step a4) the cyanhydrin silylether (V) is subjected to a coupling reaction in the sense of an intramolecular acyloin condensation under ring formation to yield the protected α-hydroxyketone (VI). A preferred base for this reaction is lithium diisopropylamide that is preferably employed in an ether solvent like tetrahydrofuran (THF). The reaction can be quenched e.g. with aqueous NH₄Cl solution. Again, the ratio of the stereoisomers with regard to the two asymmetric carbon atoms carrying the two benzene rings remains unchanged.

### Step a5)

Deprotection of the silylether with tetra-n-butylammonium fluoride (TBAF) yields the keto compound (VII). The reaction can be performed in THF as solvent and quenched with water.

In a special embodiment of the process according to the invention, if the reaction product of step a5) is obtained as a mixture of diastereomers with regard to the two asymmetric carbon atoms carrying the two benzene rings, substituted by R⁴ and F, respectively, said mixture of diastereomers is subjected to a separation.

### Step a6)

In step a6) the keto group of compound (VII) is reacted with methoxyamine hydrochloride (O-methylhydroxylamine hydrochloride) to give the oxime (VIII). The reaction can be performed in an alcoholic solution, e.g. ethanol, in the presence of a base, e.g. pyridine.

### Step a7)

Reduction of the oxime compound (VIII) to obtain the amine compound (IX) can be performed using borane and borane complexes, preferably borane-tetrahydrofuran complex.

### Step a7.1) (Optional, if R⁴ is CN)

In step a7.1) compound (IX) is reacted with a protecting group, in particular benzyl carbonochloridate to give the compound (IX′).

### Step a7.2) (Optional, if R⁴ is CN)

The compound (IX′) is reacted with dicyanozinc. R⁴′ is replaced by CN. The cleavage of the protecting group, in particular Cbz group is followed to give the compound (IX″).

### Step a8)

In a first embodiment (step a8.1), the amine compound (IX) obtained in step a7) is subjecting to a reaction with a compound of the formula (X.1) R¹—C(═O)—X, wherein X is a leaving group, preferably selected from Cl, Br, O-benzyl, CH₃SO₃ and CF₃SO₃, to give the compound of the formula (I).

In a second embodiment (step a8.2), the amine compound (IX) obtained in step a7) is subjecting to a reaction with an isocyanate of the formula (X.2) R²—N═C═O to give a compound of the formula (I), wherein R¹ is a group NHR², wherein R² is selected from C₁-C₄ alkyl and C₃-C₇ cycloalkyl, wherein alkyl is unsubstituted or substituted by 1, 2 or 3 substituents R^{a}, wherein cycloalkyl is unsubstituted or substituted by 1, 2 or 3 substituents R^{b}. The substituent R^{a} and R^{b} are as defined above. The reaction mixture can be quenched with water and purified e.g. by column chromatography.

### Step a9)

In principle, the reaction products of each of the reaction steps a2), a3), a4), a5), a6), a7), a8.1) and/or a8.2) can be subjected to one or more purification steps.

Suitable purification methods are crystallization, sublimation, extraction, chromatographic methods and combinations thereof.

A special embodiment for a purification is a separation of chiral molecules. Here the purification may be employed to obtain a product enriched in a certain stereoisomer or containing an essentially pure stereoisomer. Separation of chiral molecules may also be employed for enrichment of or pure isolation of a certain isomer from a mixture of diastereomers.

Preferably, purification of chiral molecules (I) or a precursor thereof comprises a preparative chiral chromatography. Chiral chromatography makes use of enantioselective retention mechanisms of the employed column material, in particular surface interaction and inclusion.

Suitable stationary phases for chiral separations are known in principle and can be based e.g. on oligo- and polysaccharides, such as cyclodextrins, cellulose or amylose, modified polysaccharides and cyclodextrins, polycyclic amines, copper complexes, macrocyclic glycopeptides, 1-(3,5-dinitrobenzamido)tetrahydrophenanthrene, etc.

In one embodiment, purification or separation of chiral compounds (I) or a precursor thereof is effected by high performance liquid chromatography (HPLC). In another embodiment, purification or separation of chiral compounds (I) or a precursor thereof is effected by supercritical fluid chromatography (SFC). SFC is a chromatography similar to HPLC that uses a supercritical fluid as the mobile phase. Preferably, the mobile phase comprises carbon dioxide, alcohols, acetonitrile, dichloromethane, chloroform, ethyl acetate and mixtures thereof. Preferred alcohols used as mobile phase are selected from methanol, ethanol, isopropyl alcohol and mixtures thereof. In a special embodiment, carbon dioxide or mixtures of carbon dioxide and at least one further solvent are used as mobile phase. In principle, in SFC the same columns can be employed as in standard HPLC systems. In HPLC and SFC techniques, a variety of detection methods may be used, e.g. UV/VIS spectroscopy, mass spectrometry, FID, evaporative light scattering, etc.

Compounds of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be in particular prepared by the methods illustrated in the following schemes. Concrete conditions for the reactions can be taken from the following examples.

Any compound of formula (I) may be produced in particular by the depicted schemes by suitable selection of reagents with appropriate substitution. Solvents, temperatures, pressures, and other reaction conditions may readily be selected by one of ordinary skill in the art. Starting materials are commercially available or can be readily prepared by one of ordinary skill in the art. Constituents of compounds are as defined herein or elsewhere in the specification.

General routes to compounds described in the invention are illustrated in schemes 1, 2, 3 and 4, where the R¹ and R⁴ has one of the meanings as defined previously in the text or a functional group that can be converted to the desired final substituent and X is halogen or a leaving group. The formation of the inventive compounds is accomplished by a multi-step synthetic sequence starting with compound 1. Scheme 1 describes the general route for the preparation of the starting materials. Starting from compound 1, which is subjected to a bromination reaction to form compound 2. Compound 3 is obtained by substitution of compound 2 in the presence of 3,5-dimethoxyphenol. Compound 4 is obtained by hydrolysis of compound 3. The cyclization to compound 5 is carried out in the presence of a phosphoryl chloride and tin chloride.

Scheme 2 describes the Michael addition of compound 5 to an aldehyde yields 6. The aldehyde 6 is subjected to trimethylsilyl cyanide (TMSCN) to yield in the cyanohydrin 7. Formation of ketone 8 is initiated by addition of lithium diisopropylamide (LDA), followed by deprotection of the resulting mixture with tetra-n-butylammonium fluoride (TBAF). The purification of 8 is carried out by HPLC.

Scheme 3 describes the reaction of 8 to the oxime 9. Reduction of compound 9 yields compound 10.

Scheme 4 describes the final conversion to compounds of formula (I). The compounds of formula (I) in its enantiomeric pure form are finally obtained via chiral HPLC separation.

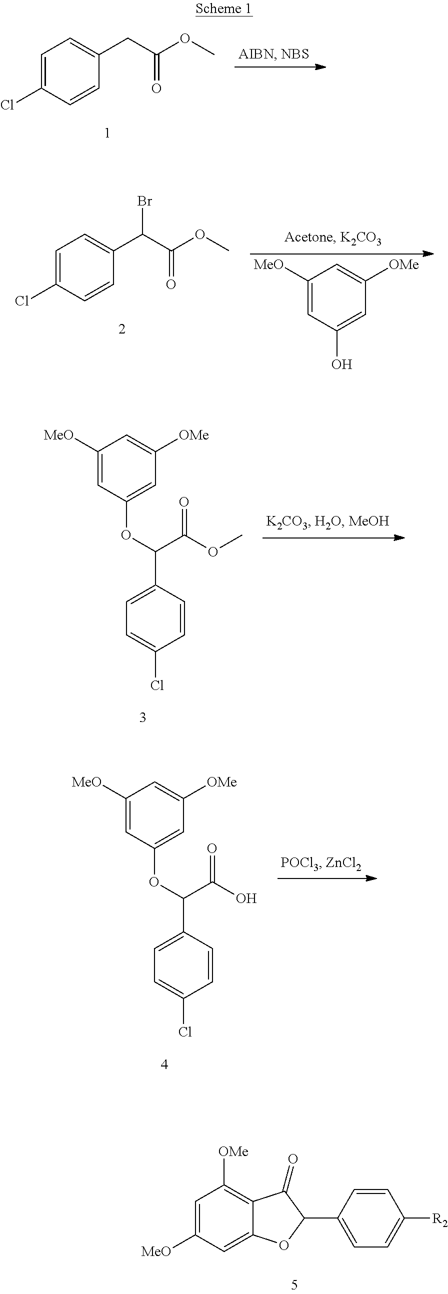

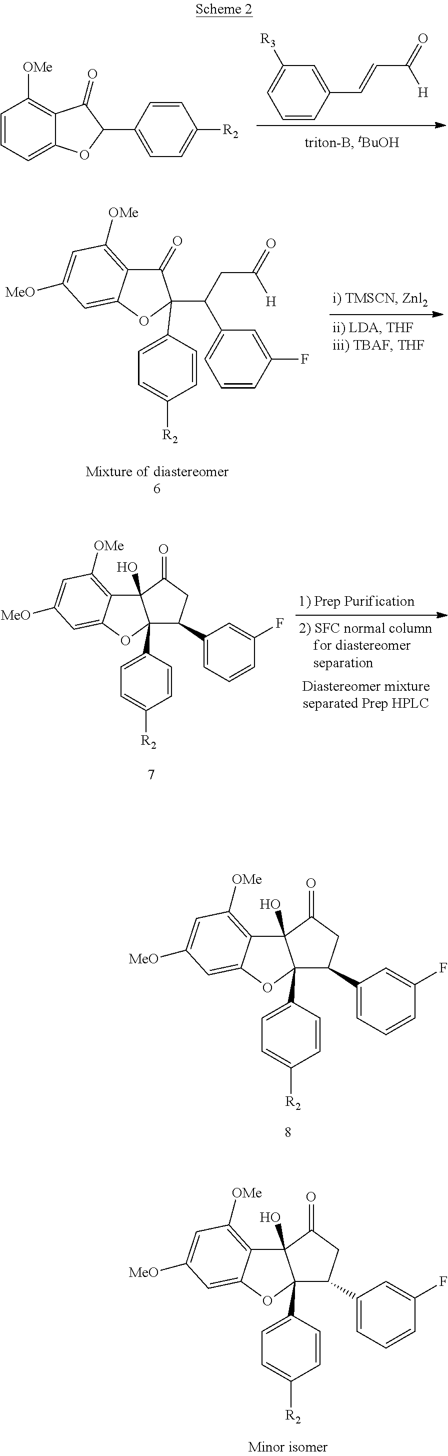

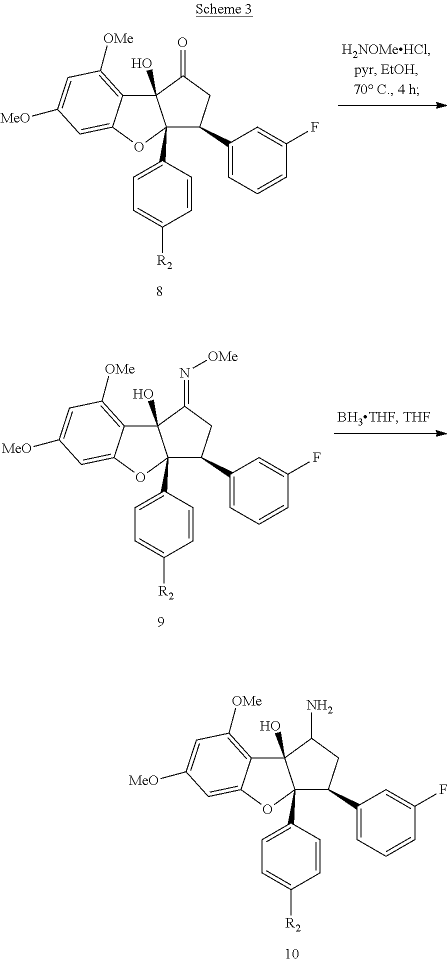

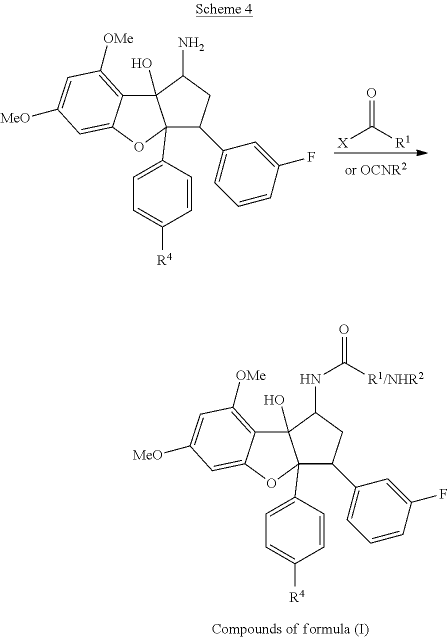

### Pharmaceutical Composition

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “therapeutically effective” is intended to qualify the amount of each agent, which will achieve the goal of improvement in disorder severity and the frequency of incidence, while avoiding adverse side-effects typically associated with alternative therapies. For example, effective anticancer agents prolong the survivability of the patient or his/her life quality, inhibit the rapidly proliferating cell growth associated with the neoplasm, or effect a regression of the neoplasm.

The terms “treat,” “treating,” and “treatment,” as used herein, refer to any type of intervention or process performed on, or administering an active agent to, the subject with the objective of reversing, alleviating, ameliorating, inhibiting or slowing down or preventing the progression, development, severity or recurrence of a symptom, complication, condition or biochemical indicia associated with a disease. By contrast, “prophylaxis” or “prevention” refers to administration to a subject who does not have a disease to prevent the disease from occurring.

As used herein, the term “cell” is meant to refer to a cell that is in vitro, ex vivo or in vivo. In the sense of the invention, an ex vivo cell can be part of a tissue sample excised from an organism such as a mammal. In the sense of the invention, an in vitro cell can be a cell in a cell culture. In the sense of the invention, an in vivo cell is a cell living in an organism such as a mammal.

The term “patient” includes humans and animals that receive either therapeutic or prophylactic treatment.

The term “subject” includes any human or animal. For example, the methods and compositions herein disclosed can be used to treat a subject having cancer.

A (non-human) animal includes all vertebrates, e.g. mammals and non-mammals, including cows, sheep, pigs, goats, horses, poultry, dogs, cats, non-human primates, rodents etc. In one embodiment, the subject is a human subject.

The phrase “pharmaceutically acceptable carrier” as used herein means a pharmaceutically acceptable material, composition or vehicle, such as a liquid or solid diluent, solvent, excipient, manufacturing aid (e.g. lubricant) or encapsulating material, involved in carrying or transporting the subject compound from one organ, or portion of the body, to another organ, or portion of the body. Each carrier must be “acceptable” in the sense of being compatible with the other ingredients of the formulation.

Suitable other ingredients are the afore-mentioned carrier and further additives, including adjuvants, preserving agents, fillers, flow regulating agents, disintegrating agents, wetting agents, emulsifying agents, suspending agents, sweetening agents, flavoring agents, bittering agents, perfuming agents, antibacterial agents, antifungal agents, lubricating agents, dispensing agents, etc. Suitable additives are selected depending on the nature of the mode of administration and dosage forms; and not injurious to the patient.

The term “pharmaceutical composition” means a composition comprising a compound of the invention in combination with at least one further compound selected from
a) at least one further pharmaceutically active substance and
b) at least one additional pharmaceutically acceptable carrier and or additive.

### RAS

The term “RAS inhibitor” refers to an agent capable of decreasing RAS protein levels, decreasing RAS activity levels and/or inhibiting RAS expression levels in the cells. The RAS inhibitor may be a reversible or irreversible inhibitor. As used herein, “RAS” protein refers to a protein that is a member of a family of related proteins that are expressed in all human and animal cell lineages and organs. All RAS protein family members belong to a class of proteins called small GTPase (also known as small G proteins, a family of hydrolase enzymes that can bind and hydrolyse GTP), and are involved in transmitting signals within cells (cellular signal transduction). RAS is the prototypical member of the RAS superfamily of proteins, which are all related in three-dimensional structure and regulate diverse cell behaviours. When RAS is ‘switched on’ by incoming signals, it subsequently switches on other proteins, which ultimately turn on genes involved in cell growth, differentiation, and survival. Mutations in RAS genes can lead to the production of permanently activated RAS proteins, which can cause unintended and overactive signaling inside the cell, even in the absence of incoming signals. Because these signals result in cell growth and division, overactive RAS signaling can ultimately lead to cancer. The three RAS genes in humans (HRAS, KRAS, and NRAS) are the most common oncogenes in human cancer. As mentioned, the clinically most notable members of the RAS subfamily are HRAS, KRAS and NRAS. However, there are other members of this subfamily, which are e.g. selected from DIRAS1, DIRAS2, DIRAS3, ERAS, GEM, MRAS, NKIRAS1, NKIRAS2, NRAS, RALA, RALB, RAP1A, RAP1B, RAP2A, RAP2B, RAP2C, RASD1, RASD2, RASL10A, RASL10B, RASL11A, RASL11B, RASL12, REM1, REM2, RERG, RERGL, RRAD, RRAS, RRAS2.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and a pharmaceutical composition comprising in at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be administered to humans and animals, preferably humans.

In principle any method of administration may be used to deliver the compound or pharmaceutical composition according to the invention to a subject. Suitable methods of administration are orally, enterally, parenterally, intravenously, topically, intramuscular, subcutaneous/dermal routes.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can selectively decrease RAS protein levels, decrease RAS activity levels and/or inhibit RAS expression levels in the cells. For example, the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can be used to selectively decrease RAS activity levels and/or inhibit RAS expression levels in cells or in an individual in need of a decrease in RAS protein levels, decrease in RAS activity levels and/or inhibition of RAS expression levels by administering an inhibiting amount of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above or a salt thereof.

In one embodiment, the present invention provides a combined preparation of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or a pharmaceutically acceptable salt thereof, and an additional therapeutic agent(s) for simultaneous, separate or sequential use in the treatment and/or prophylaxis of multiple diseases, preferably of proliferative disorders (e.g. cancer), in particular disorders associated with the activity of RAS protein.

Additional therapeutic agent(s) are selected from chemotherapeutic agents, radiotherapeutic agents, immuno-oncology agents, and combinations thereof.

In one aspect, the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above are sequentially administered prior to administration of the immuno-oncology agent. In another aspect, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above are administered concurrently with the immuno-oncology agent. In yet another aspect, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above are sequentially administered after administration of the immuno-oncology agent.

In another aspect, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be co-formulated with an immuno-oncology agent.

Immuno-oncology agents include, for example, a small molecule drug, antibody or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Suitable of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTpR, LIGHT, DcR3, HVEM, VEGETLiA, TRAMP/DR3, EDAR, EDA1, XEDAR, EDA2, TNFR1, Lymphotoxin a/TNFp, TNFR2, TNFa, LTpR, Lymphotoxin a 1b2, FAS, FASL, RELT, DR6, TROY, NGFR.

In one aspect, T cell responses can be stimulated by a combination of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and one or more of
(i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and
(ii) an agonist of a protein that stimulates T cell activation such as B7-1, B7-2, CD28, 4-1BB (CD 137), 4-1BBL, ICOS, ICOS-L, OX40, OX40L, GITR, GITRL, CD70, CD27, CD40, DR3 and CD28H.

Other agents that can be combined with compound(s) of formulae (I), (I-A), (I.a′), (I-A.a′), (I.a), (I-A.a), (A), (B), (C), (D), (E), (F), (G), (H), ( ), (J), (K), (L), (M), (N), (O), (P), (Q), (R), (S), (T), (U), (V) and an enantiomeric mixture as defined above for the treatment of cancer include antagonists of inhibitory receptors on NK cells or agonists of activating receptors on NK cells. For example, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can be combined with antagonists of KIR, such as Lirilumab.

Yet other agents for combination therapies include agents that inhibit or deplete macrophages or monocytes, including but not limited to CSF-1R antagonists such as CSF-1R antagonist antibodies including RG7155.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy can also embrace the administration of the therapeutic agents as described above in further combination with other biologically active ingredients and non-drug therapies (e.g surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Types of cancers that may be treated with the compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above include, but are not limited to, prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, haematological malignancies (including blood, bone marrow and lymph nodes) or testicular seminoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above for treatment of RAS protein associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may also be combined with immunotherapy drugs, including cytokines such as interferon alpha, interleukin 2, and tumor necrosis factor (TNF) in the treatment of melanoma. Compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), NV) and an enantiomeric mixture as defined above may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compound(s) of formulae (I), (I.a), (I.b, (A), (B), (C), (D), (E), (F), (G), (H), (I), (J), (K), (L), (M), (N), (O), (P), (Q), (R), (S), (T), (U), (V) and an enantiomeric mixture as defined, using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C.

Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-α), etoposide, and teniposide.

Other cytotoxic agents include navelbene, CPT-11, anastrazole, letrazole, capecitabine, reloxafme, and droloxafme.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (HERCEPTIN®), antibodies to costimulatory molecules such as CTLA-4, 4-1BB and PD-1, or antibodies to cytokines (IL-IO or TGF-b).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

In a specific embodiment of the present invention, at least one compound of formulae (I), (I.a), (I.b, (A), (B), (C), (D), (E), (F), (G), (H), (I), (J), (K), (L), (M), (N), (O), (P), (Q), (R), (S), (T), (U), (V) and an enantiomeric mixture as defined above and at least one chemotherapeutic agent are administered to the patient concurrently or sequentially. In other words, at least one compound(s) of formulae (I), (I.a), (I.b, (A), (B), (C), (D), (E), (F), (G), (H), (I), (J), (K), (L), (M), (N), (O), (P), (Q), (R), (S), (T), (U), (V) and an enantiomeric mixture as defined above may be administered first, at least one chemotherapeutic agent may be administered first, or at least one compound of formulae (I), (I.a), (I.b, (A), (B), (C), (D), (E), (F), (G), (H), (I), (J), (K), (L), (M), (N), (O), (P), (Q), (R), (S), (T), (U), (V) and an enantiomeric mixture as defined above may be administered at the same time. Additionally, when more than one compound of formulae (I), (I.a), (I.b, (A), (B), (C), (D), (E), (F), (G), (H), (I), (J), (K), (L), (M), (N), (O), (P), (Q), (R), (S), (T), (U), (V) and an enantiomeric mixture as defined above and/or chemotherapeutic agent is used, the compounds may be administered in any order.

The invention also provides pharmaceutically compositions which comprise a therapeutically effective amount of one or more of the compound(s) of formulae (I), (I-A), (I.a′), (I-A.a′), (I.a), (I-A.a), (A), (B), (C), (D, (E), (F), (G), (H), (I), (J), (K), (L), (M), (N), (O), (P), (Q), (R), (S), (T), (U), (V) and an enantiomeric mixture as defined above, formulated together with one or more pharmaceutically acceptable carriers (additives) and/or diluents, and optionally, one or more additional therapeutic agents described above.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, may be administered by any suitable route, preferably in the form of a pharmaceutical composition adapted to such a route, and in a dose effective for the treatment intended. The compound(s) and compositions of the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g. as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or animal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, bittering agents, coloring agents, demulcents, antioxidants, and preserving agents.

A tablet can, for example, be prepared by admixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasantly tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or at least one salt thereof with at least one inert solid diluent, such as, for example, calcium carbonate; calcium phosphate; and kaolin.

Soft gelatin capsules can, for example, be prepared by mixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, hydroxypropyl-methylcellulose and hydroxypropyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof in either a vegetable oil, such as, for example, arachis oil, olive oil, sesame oil and coconut oil or in mineral oil, such as, for example, liquid paraffin. An oily suspension can also contain at least one thickening agent, such as, for example, beeswax, hard paraffin and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof can, for example, be prepared as an oil-in-water emulsion. The oily phase of the emulsions comprising compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilize) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers, including saline, dextrose, water or with cyclodextrin solubilization (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above in an oily phase, such as, for example, a mixture of soybean oil and lecithin; 2) combining the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol poly ethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

The amounts of compounds that are administered and the dosage regimen for treating a disease condition with the compounds and/or compositions of this invention depend on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

Pharmaceutical compositions of this invention comprise at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof, and optionally an additional agent selected from any pharmaceutically acceptable carrier, adjuvant, and vehicle. Alternate compositions of this invention comprise a compound of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, or a prodrug thereof, and a pharmaceutically acceptable carrier, adjuvant, or vehicle.

The present invention also includes pharmaceutical kits useful, for example, in the treatment or prevention of RAS protein-associated diseases. Thus, the present invention also relates to a kit containing a formulation comprising: a) a pharmaceutical composition comprising a compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, or a therapeutically acceptable salt thereof and a pharmaceutically acceptable carrier; and b) instructions for dosing of the pharmaceutical composition for the treatment of a disorder in which inhibition of RAS activation is effective in treating the disorder.

Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compound(s) of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g. oral tablets, capsules, elixirs, and syrups, and consistent with conventional pharmaceutical practices. Dosage forms (pharmaceutical compositions) suitable for administration may contain from about 1 milligram to about 200 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compound of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture (250 mg), lactose (75 mg), and magnesium stearate (15 mg). The mixture is passed through a 60 mesh sieve and packed into a no. 1 gelatin capsule.

A typical injectable preparation is produced by aseptically placing at least one of the compound of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, alone or in combination with a pharmaceutical carrier. Optionally, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can be used alone, in combination with other compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, or in combination with one or more other therapeutic agent(s), e.g. an anticancer agent or other pharmaceutically active material.

Regardless of the route of administration selected, the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above employed in the pharmaceutical composition at levels lower than that required in order to achieve the desired therapeutic effect and gradually increase the dosage until the desired effect is achieved.

In general, a suitable daily dose of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above will be that amount of the compound which is the lowest dose effective to produce a therapeutic effect. Such an effective dose will generally depend upon the factors described above. Generally, oral, intravenous, intracerebroventricular and subcutaneous doses of the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day.

While it is possible for compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above to be administered alone, it is preferable to administer the compound as a pharmaceutical formulation (composition).

The above other therapeutic agents, when employed in combination with the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### Eukaryotic Ignition Factor 4A

The mechanisms governing the basic subsistence of eukaryotic cells are immensely complex; it is therefore unsurprising that regulation occurs at a number of stages of protein synthesis. Human translational control is of increasing research interest as it has connotations in a range of diseases. Orthologs of many of the factors involved in human translation are shared by a range of eukaryotic organisms. Synthesis of protein from mature messenger RNA in eukaryotes is divided into translation initiation, elongation, and termination of these stages; the initiation of translation is the rate limiting step. Within the process of translation initiation; the bottleneck occurs shortly before the ribosome binds to the 5′ m7GTP facilitated by a number of proteins; it is at this stage that constrictions born of stress, amino acid starvation etc. take effect.

Eukaryotic initiation factor complex 2 (eIF2) forms a ternary complex with GTP and the initiator Met-tRNA—this process is regulated by guanine nucleotide exchange and phosphorylation and serves as the main regulatory element of the bottleneck of gene expression. Before translation can progress to the elongation stage, a number of initiation factors must facilitate the synergy of the ribosome and the mRNA and ensure that the 5′ UTR of the mRNA is sufficiently devoid of secondary structure. Binding in this way is facilitated by group 4 eukaryotic initiation factors; eIF4F has implications in the normal regulation of translation as well as the transformation and progression of cancerous cells.

eIF4F is responsible for the binding of capped mRNA to the 40S ribosomal subunit via eIF3. The mRNA cap is bound by eIF4E (25 kDa), eIF4G (185 kDa) acts as a scaffold for the complex whilst the ATP-dependent RNA helicase eIF4A (46 kDa) processes the secondary structure of the mRNA 5′ UTR to render it more conducive to ribosomal binding and subsequent translation. Together these three proteins are referred to as eIF4F. For maximal activity; eIF4A also requires eIF4B (80 kDa), which itself is enhanced by eIF4H (25 kDa). Once bound to the 5′ cap of mRNA, this 48S complex then searches for the (usually) AUG start codon and translation begins.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and a pharmaceutical composition comprising in at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be administered to humans and animals, preferably humans.

In principle any method of administration may be used to deliver the compound or pharmaceutical composition according to the invention to a subject. Suitable methods of administration are orally, enterally, parenterally, intravenously, topically, intramuscular, subcutaneous routes.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can selectively decrease activity of eIF4A, decrease eIF4A activity levels and/or inhibit activity of eIF4 in the cytosol. For example, the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can be used to selectively decrease eIF4A activity levels and/or inhibit eIF4A in cytosol or in an individual in need of a decrease in activity of eIF4A, decrease in eIF4A activity and/or inhibition of eIF4A activity levels by administering an inhibiting amount of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above or a salt thereof.

In one embodiment, the present invention provides a combined preparation of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or a pharmaceutically acceptable salt thereof, and an additional therapeutic agent(s) for simultaneous, separate or sequential use in the treatment and/or prophylaxis of multiple diseases, preferably of proliferative disorders (e.g. cancer), in particular disorders associated with the activity of eIF4A.

Additional therapeutic agent(s) are selected from chemotherapeutic agents, radiotherapeutic agents, immuno-oncology agents, and combinations thereof.

In one aspect, the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above are sequentially administered prior to administration of the immuno-oncology agent. In another aspect, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above are administered concurrently with the immuno-oncology agent. In yet another aspect, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above are sequentially administered after administration of the immuno-oncology agent.

In another aspect, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be co-formulated with an immuno-oncology agent.

Immuno-oncology agents include, for example, a small molecule drug, antibody or other biologic or small molecule. Examples of biologic immuno-oncology agents include, but are not limited to, cancer vaccines, antibodies, and cytokines. In one aspect, the antibody is a monoclonal antibody. In another aspect, the monoclonal antibody is humanized or human.

In one aspect, the immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses (often referred to as immune checkpoint regulators).

Suitable of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTpR, LIGHT, DcR3, HVEM, VEGETLiA, TRAMP/DR3, EDAR, EDA1, XEDAR, EDA2, TNFR1, Lymphotoxin a/TNFp, TNFR2, TNFa, LTpR, Lymphotoxin a 1b2, FAS, FASL, RELT, DR6, TROY, NGFR.

In one aspect, T cell responses can be stimulated by a combination of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and one or more of
(i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4, and
(ii) an agonist of a protein that stimulates T cell activation such as B7-1, B7-2, CD28, 4-1BB (CD 137), 4-1BBL, ICOS, ICOS-L, OX40, OX40L, GITR, GITRL, CD70, CD27, CD40, DR3 and CD28H.

Other agents that can be combined with compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above for the treatment of cancer include antagonists of inhibitory receptors on NK cells or agonists of activating receptors on NK cells. For example, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can be combined with antagonists of KIR, such as Lirilumab.

Yet other agents for combination therapies include agents that inhibit or deplete macrophages or monocytes, including but not limited to CSF-1R antagonists such as CSF-1R antagonist antibodies including RG7155.

The combination therapy is intended to embrace administration of these therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration can be accomplished, for example, by administering to the subject a single dosage form having a fixed ratio of each therapeutic agent or in multiple, single dosage forms for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Combination therapy can also embrace the administration of the therapeutic agents as described above in further combination with other biologically active ingredients and non-drug therapies (e.g surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

Types of cancers that may be treated with the compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above include, but are not limited to, prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, haematological malignancies (including blood, bone marrow and lymph nodes) or testicular seminoma.

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2 and GM-CSF), and/or tyrosine kinase inhibitors can be optionally used in combination with the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above for treatment of eIF4A associated diseases, disorders or conditions. The agents can be combined with the present compounds in a single dosage form, or the agents can be administered simultaneously or sequentially as separate dosage forms.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (CYTOXAN®), ifosfamide, melphalan, chlorambucil, pipobroman, triethylene-melamine, triethylenethiophosphoramine, busulfan, carmustine, lomustine, streptozocin, dacarbazine, and temozolomide.

In the treatment of melanoma, suitable agents for use in combination with the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined include: dacarbazine (DTIC), optionally, along with other chemotherapy drugs such as carmustine (BCNU) and cisplatin; the “Dartmouth regimen”, which consists of DTIC, BCNU, cisplatin and tamoxifen; a combination of cisplatin, vinblastine, and DTIC, temozolomide or YERVOY™. Compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may also be combined with immunotherapy drugs, including cytokines such as interferon alpha, interleukin 2, and tumor necrosis factor (TNF) in the treatment of melanoma. Compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with a combination of agents including one or more compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined, using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 38.9° C. to 40° C. Melphalan is the drug most often used in this chemotherapy procedure. This can be given with another agent called tumor necrosis factor (TNF).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (Taxol), mithramycin, deoxyco-formycin, mitomycin-C, L-asparaginase, interferons (especially IFN-a), etoposide, and teniposide.

Other cytotoxic agents include navelbene, CPT-11, anastrazole, letrazole, capecitabine, reloxafme, and droloxafme.

Also suitable are cytotoxic agents such as epidophyllotoxin; an antineoplastic enzyme; a topoisomerase inhibitor; procarbazine; mitoxantrone; platinum coordination complexes such as cisplatin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (HERCEPTIN®), antibodies to costimulatory molecules such as CTLA-4, 4-1BB and PD-1, or antibodies to cytokines (IL-IO or TGF-b).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

In a specific embodiment of the present invention, at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and at least one chemotherapeutic agent are administered to the patient concurrently or sequentially. In other words, at least one compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be administered first, at least one chemotherapeutic agent may be administered first, or at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be administered at the same time. Additionally, when more than one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or chemotherapeutic agent is used, the compounds may be administered in any order.

The invention also provides pharmaceutically compositions which comprise a therapeutically effective amount of one or more of the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, formulated together with one or more pharmaceutically acceptable carriers (additives) and/or diluents, and optionally, one or more additional therapeutic agents described above.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, may be administered by any suitable route, preferably in the form of a pharmaceutical composition adapted to such a route, and in a dose effective for the treatment intended. The compound(s) and compositions of the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, can be administered for any of the uses described herein by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g. as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 0.1 to 1000 mg, preferably from about 0.25 to 250 mg, and more preferably from about 0.5 to 100 mg. A suitable daily dose for a human or animal may vary widely depending on the condition of the patient and other factors, but, can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, bittering agents, coloring agents, demulcents, antioxidants, and preserving agents.

A tablet can, for example, be prepared by admixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasantly tasting drug, or delay disintegration and absorption of the active ingredient in the gastrointestinal tract thereby sustaining the effects of the active ingredient for a longer period. Exemplary water soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or at least one salt thereof with at least one inert solid diluent, such as, for example, calcium carbonate; calcium phosphate; and kaolin.

Soft gelatin capsules can, for example, be prepared by mixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, hydroxypropyl-methylcellulose and hydroxypropyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof in either a vegetable oil, such as, for example, arachis oil, olive oil, sesame oil and coconut oil or in mineral oil, such as, for example, liquid paraffin. An oily suspension can also contain at least one thickening agent, such as, for example, beeswax, hard paraffin and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof can, for example, be prepared as an oil-in-water emulsion. The oily phase of the emulsions comprising compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilize) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions and aqueous or oleaginous suspensions.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers, including saline, dextrose, water or with cyclodextrin solubilization (i.e. Captisol), cosolvent solubilization (i.e. propylene glycol) or micellar solubilization (i.e. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above in an oily phase, such as, for example, a mixture of soybean oil and lecithin; 2) combining the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V. Jr. et al. Remington: The Science and Practice of Pharmacy (2 Volumes), 22nd Edition (2012), Pharmaceutical Press.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol poly ethyleneglycol 1000 succinate, surfactants used in pharmaceutical dosage forms such as Tweens, polyethoxylated castor oil such as CREMOPHOR surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

The amounts of compounds that are administered and the dosage regimen for treating a disease condition with the compounds and/or compositions of this invention depend on a variety of factors, including the age, weight, sex, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.0025 and about 50 mg/kg body weight and most preferably between about 0.005 to 10 mg/kg body weight, may be appropriate. The daily dose can be administered in one to four doses per day. Other dosing schedules include one dose per week and one dose per two day cycle.

Pharmaceutical compositions of this invention comprise at least one compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above and/or at least one pharmaceutically acceptable salt thereof, and optionally an additional agent selected from any pharmaceutically acceptable carrier, adjuvant, and vehicle. Alternate compositions of this invention comprise a compound of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, or a prodrug thereof, and a pharmaceutically acceptable carrier, adjuvant, or vehicle.

The present invention also includes pharmaceutical kits useful, for example, in the treatment or prevention of eIF4a-associated diseases. Thus, the present invention also relates to a kit containing a formulation comprising: a) a pharmaceutical composition comprising a compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, or a therapeutically acceptable salt thereof and a pharmaceutically acceptable carrier; and b) instructions for dosing of the pharmaceutical composition for the treatment of a disorder in which inhibition of the activity eIF4A, is effective in treating the disorder.

Such kits can further include, if desired, one or more of various conventional pharmaceutical kit components, such as, for example, containers with one or more pharmaceutically acceptable carriers, additional containers, as will be readily apparent to those skilled in the art. Instructions, either as inserts or as labels, indicating quantities of the components to be administered, guidelines for administration, and/or guidelines for mixing the components, can also be included in the kit.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.001 to about 5000 mg per day, preferably between about 0.01 to about 1000 mg per day, and most preferably between about 0.1 to about 250 mg per day. Intravenously, the most preferred doses will range from about 0.01 to about 10 mg/kg/minute during a constant rate infusion. Compound(s) of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g. oral tablets, capsules, elixirs, and syrups, and consistent with conventional pharmaceutical practices. Dosage forms (pharmaceutical compositions) suitable for administration may contain from about 1 milligram to about 200 milligrams of active ingredient per dosage unit. In these pharmaceutical compositions the active ingredient will ordinarily be present in an amount of about 0.1-95% by weight based on the total weight of the composition.

A typical capsule for oral administration contains at least one of the compound of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture (250 mg), lactose (75 mg), and magnesium stearate (15 mg). The mixture is passed through a 60 mesh sieve and packed into a no. 1 gelatin capsule.

A typical injectable preparation is produced by aseptically placing at least one of the compound of the formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture (250 mg) into a vial, aseptically freeze-drying and sealing. For use, the contents of the vial are mixed with 2 mL of physiological saline, to produce an injectable preparation.

The present invention includes within its scope pharmaceutical compositions comprising, as an active ingredient, a therapeutically effective amount of at least one of the compound of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, alone or in combination with a pharmaceutical carrier. Optionally, compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above can be used alone, in combination with other compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, or in combination with one or more other therapeutic agent(s), e.g. an anticancer agent or other pharmaceutically active material.

Regardless of the route of administration selected, the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above employed in the pharmaceutical composition at levels lower than that required in order to achieve the desired therapeutic effect and gradually increase the dosage until the desired effect is achieved.

In general, a suitable daily dose of compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above will be that amount of the compound which is the lowest dose effective to produce a therapeutic effect. Such an effective dose will generally depend upon the factors described above. Generally, oral, intravenous, intracerebroventricular and subcutaneous doses of the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above for a patient will range from about 0.01 to about 50 mg per kilogram of body weight per day.

If desired, the effective daily dose of the active compound may be administered as two, three, four, five, six or more sub-doses administered separately at appropriate intervals throughout the day, optionally, in unit dosage forms. In certain aspects of the invention, dosing is one administration per day, every other day, twice per week or per week.

While it is possible for compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above to be administered alone, it is preferable to administer the compound as a pharmaceutical formulation (composition).

The above other therapeutic agents, when employed in combination with the compound(s) of formulae (I), (I-A), (I.a′), (I-A.a′), (I.a), (I-A.a), (A), (B), (C), (D), (E), (F), (G), (H), (I), (J), (K), (L), (M), (N), (O), (P), (Q), (R), (S), (T), (U), (V) and an enantiomeric mixture as defined above, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

The invention will be illustrated further with reference to the examples that follow, without restricting the scope to the specific embodiments described. The invention includes all combinations of described and especially of preferred features that do not exclude each other.

### DESCRIPTION OF THE DRAWINGS

FIG. 1: NanoBiT assay for RAS activation (KRAS G12V, NRAS G12V and HRAS G12V)

FIG. 2: MTT assay for cell viability in 96 well cell culture plate Cells with the KRAS mutations were treated with compounds for 48 h and cell viability was evaluated by MTT assay. The bars represent mean±SEM from 3 independent experiments. The dot plot indicate the value from each experiment.

FIG. 3: MTT assay for the growth of different tumour cell lines with RAS mutations in soft agar with the listed compounds. Shown are data from three independent experiments.

ASPC-1, NCI-2122, HCT-116, and MDA-MB-231 cells were used for the soft agar colony formation assay. After the treatment with compounds (25 nM), the colonies were stained with MTT and the value was quantified by measuring absorbance at 570 nm after solubilization. The bars represent mean±SD from 3 independent experiments. The dot plot indicates the value from each.

FIG. 4: NanoBit assay for RAS activation (KRAS G12, KRAS G13, KRAS Q61)

NanoBiT assay for the RAS GTP-loading was performed in HeLa cells transfected with LgBit-KRAS mutants and SmBit-CRAF-RBD. Cells were treated with compounds according to the invention (250 nM) for 2 h in serum-free DMEM. After incubation, the substrate for NanoLuc was added, and the luminescence was measured in a multiplate reader. Data were normalized to cells transfected with the indicated mutant and exposed to DMSO for 2 h. DMSO-treated cells were set as 1. The bars represent mean±SEM from 3 independent experiments.

FIG. 5: Plasmid MAP of the dual luciferase assay system

FIG. 6: Dual luciferase assay for cap dependent translation initiation (eIF4A)

FIG. 7: NanoBiT assay for RAS activation (KRAS G12V, NRAS G12V and HRAS G12V)

FIG. 8: Nanobit assay to measure activation of different KRAS mutants

FIG. 9: MTT assay for cell growth of different RAS mutated cancer cell lines in soft agar

FIG. 10: NanoBiT assay for RAS activation (KRAS G12V, NRAS G12V and HRAS G12V)

FIG. 11: Nano bit assay to measure activation of different KRAS mutations

FIG. 12: MTT assay measuring the growth of different tumour cell lines with RAS mutations

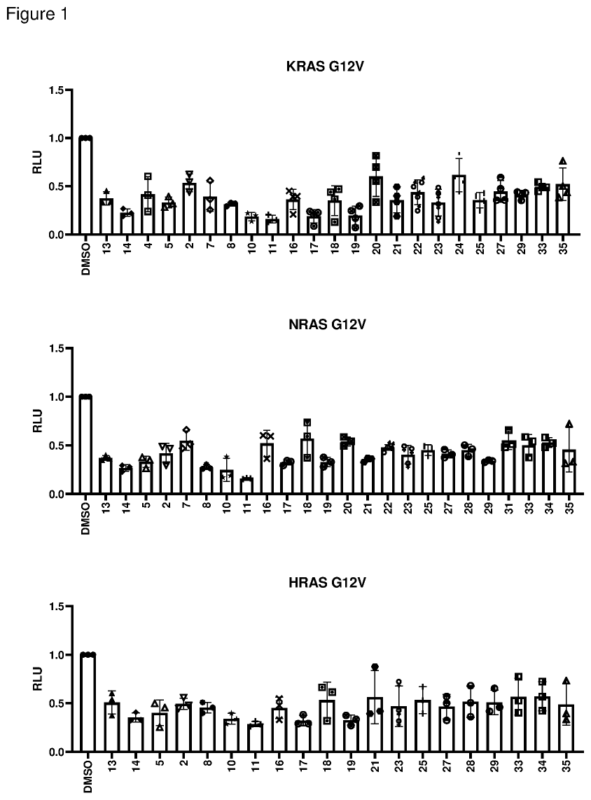

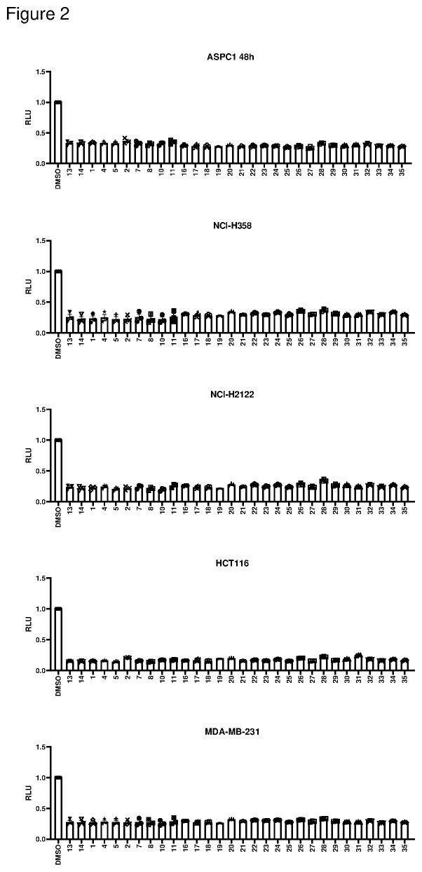

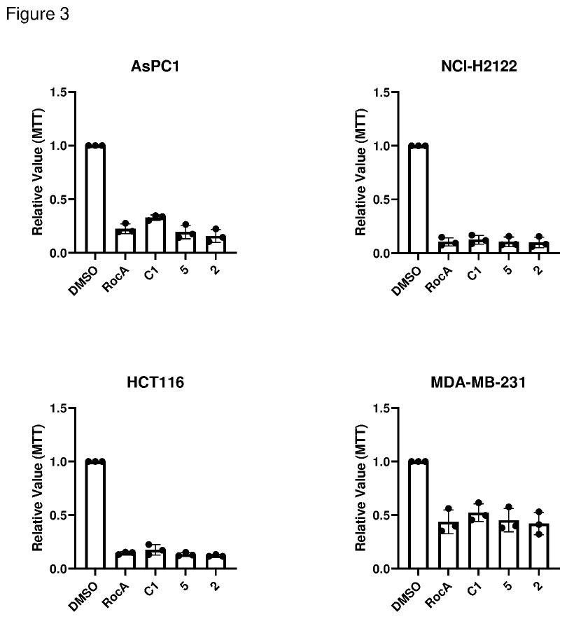

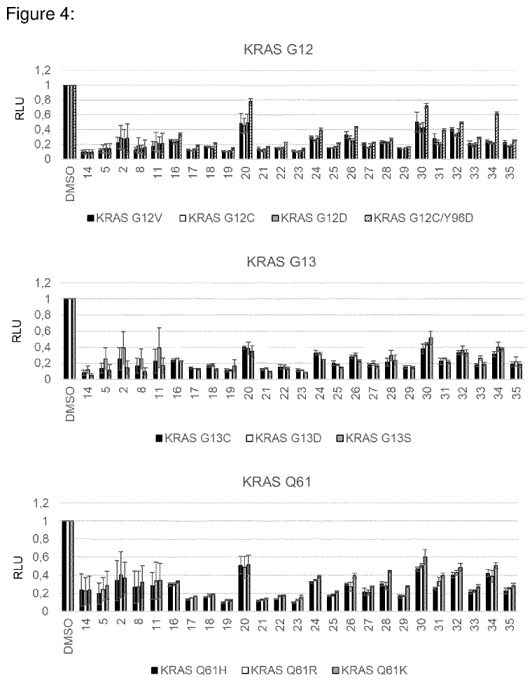

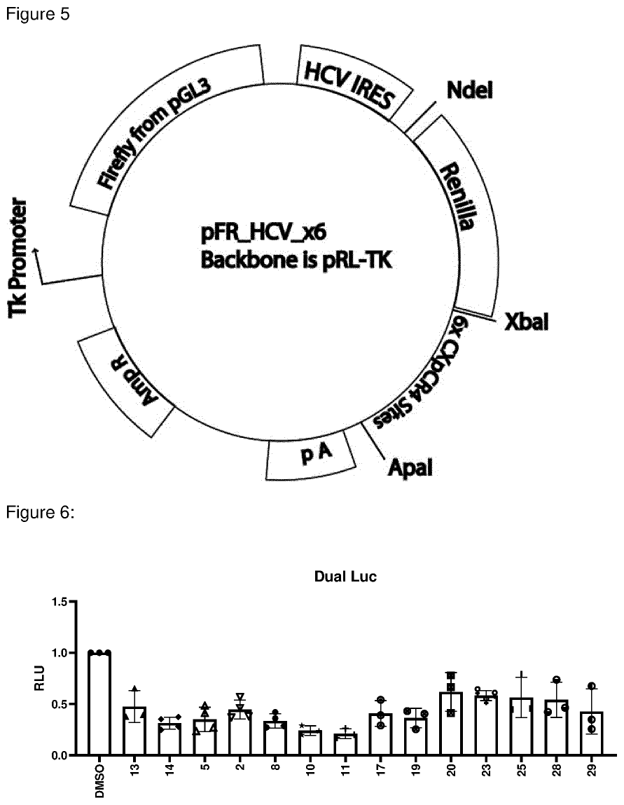

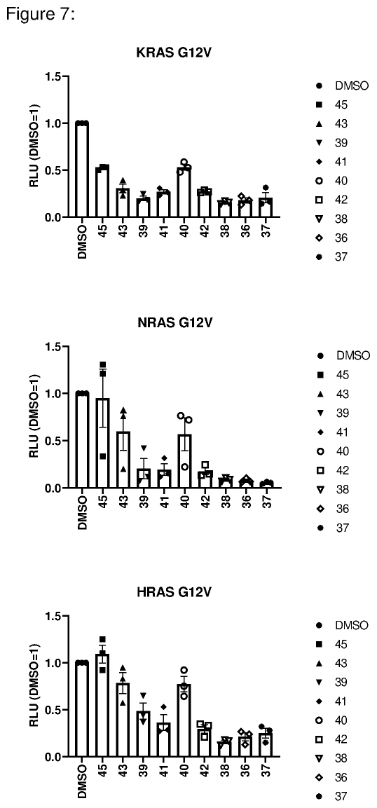

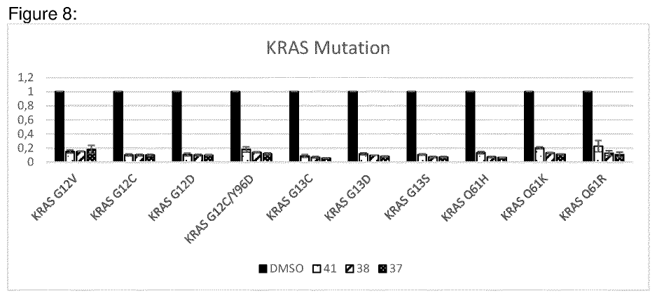

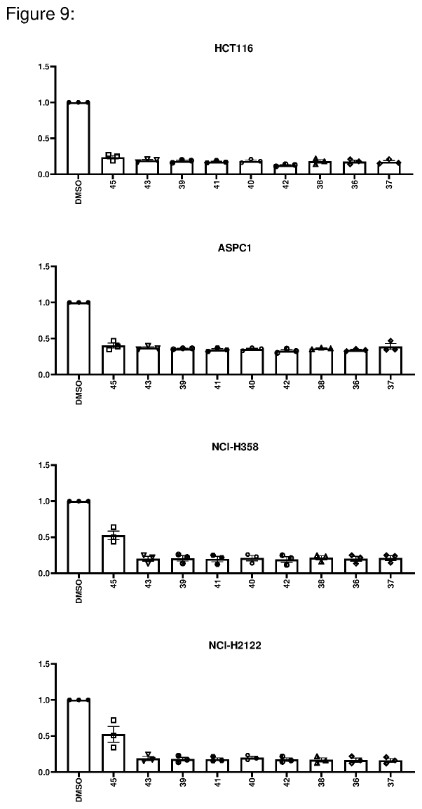

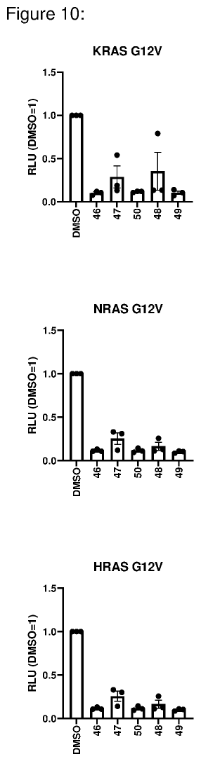

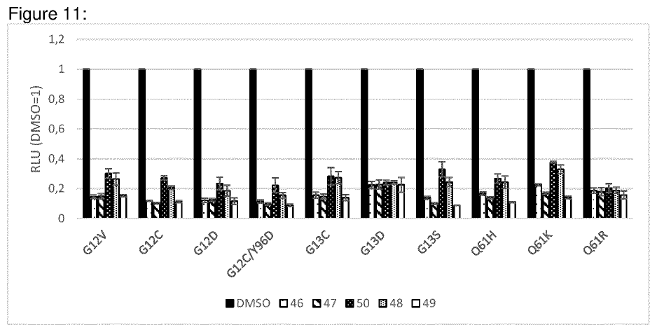

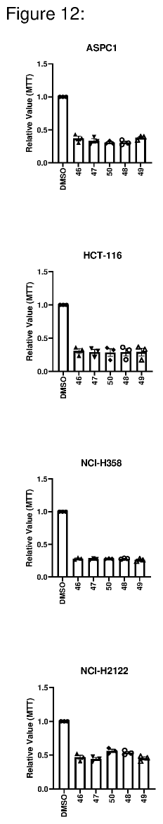

Dual luciferase assay for Cap-dependent translation initiation was performed in HeLa cells transfected with pFR_HCV_xb. Cells were treated with compounds according to the invention (100 nM) for 24 h in serum-free DMEM. After incubation, Dual luciferase reporter assay was performed according to the manufacturer's instruction and the luminescence was measured using a multiplate reader. Data were normalized to cells transfected with the indicated mutant and exposed to DMSO for 24 h. DMSO-treated cells were set as 1. The bars represent mean±SD from 3 independent experiments. The dot plot indicate the value from each experiment.

### EXAMPLES

### Abbreviations

ACN acetonitrile
AlBN azo-bis-(isobutylonitrile)
DCE Dichloroethane
DMEM Dulbecco's modified Eagle's Medium
DCM dichloromethane
DIPEA Diisopropylethylamine
DMAP Dimethylaminopyridine
DMSO dimethylsulfoxid
FA Formic acid
FBS fetal bovine serum
IPA Isopropyl alcohol
LDA lithium diisopropylamide
NBS N-bromosuccinimide
PBS phosphate-buffered saline
PE Pet-ether
RLU relative light unit (luciferase activity)/relative luciferase units
SD standard deviation
SEM trimethylsilylethoxymethyl
TBAF tetra-n-butylammonium fluoride
TFA Trifluoroacetic acid
THE tetrahydrofuran
TLC thin-layer chromatography
TMSCN trimethylsilyl cyanide
Triton B benzyltrimethylammonium hydroxide

### Cell Culture

HeLa S3 (DSMZ) were authenticated by Eurofin genomics and cultured in DMEM (10% heat inactivated FBS). HCT116 and MDA-MB-231 cells were cultured in DMEM supplemented with 10% heat inactivated fetal bovine serum (FBS). H2122 cells and AsPC-1 cells were cultured in RPMI-1640 (10% heat inactivated FBS).

### NanoBiT Assay

N-terminal LgBiT and C-terminal SmBiT construct was purchased from Promega and K, N and HRAS G12V (full length) was cloned with Xho I and Bgl I to LgBit and CRAF Ras binding domain (1-149) was cloned with EcoRI and Bgl II to SmBit. For transfections, 1 μg or 2 μg of (12 well/6 well) plasmids were transfected into cells with 0.5 mM of PEI reagent in 100 μl or 200 μl PBS. One day after transfection, cells were harvested and seeded into 96 well white plates (Greiner) or 384 well white plate (Greiner). After an additional day the medium was changed to serum free DMEM and the cells were incubated for 2 h with the compounds according to the invention. NanoGlo assay was performed according to the manufacturer's instructions. The luminescence was measured using Tecan infinite (Tecan).

### Cell Viability Assay (MTT)

Metabolic activity was quantified using Cell Proliferation Kit I (Roche, Basel, Switzerland). Cells were seeded in 96-well cell culture plates, using 5000 cells per well for HCT116, H2122 and AsPC1, and 10⁴ cells per well for MDA-MB-231 cells. After 24 h, cells were treated for 24 or 48 hours with the compounds diluted in complete medium, in triplicates. After treatment, 10 μl of MTT solution was added and incubated for 3 h in CO₂ incubator. Then 100 μl of solubilization buffer was added to each well and incubated overnight in CO₂ incubator. Cell viability, assessed by the amount of metabolized MTT, was quantified by measuring absorbance at 570 nm. IC50 calculations by non-linear regression were conducted with GraphPad Prism 5.0a.

### Soft Agar Colony Formation Assay

Agarose solution of 1.5% was mixed with equal volume of 2× growth medium supplemented with 20% FBS. The resulting agarose/medium solution was dispensed in a 96-well plate, using 50 μl per well, and incubated at room temperature for 10 min to allow bottom layer to solidify. To each well a 75 μl upper layer was added containing 5000 cells per well in 0.5% agarose/1× complete medium. After solidification of the upper layer, 125 μl of 2× compound dilution was added to each well. The cells seeded in soft agar were cultured for 5 to 10 days to allow formation of colonies.

Cell viability was then assessed using Cell Proliferation Kit I (Roche, Basel, Switzerland), by adding 25 μl MTT solution and incubating for 4 h in CO₂ incubator. Then medium was removed and 175 μl of solubilization buffer was added to each well and incubated 1 h at 70° C. Once agar was melted, cell viability, assessed by the amount of metabolized MTT, was quantified by measuring absorbance at 570 nm.

### LCMS:

Instrument name: Agilent Technologies 1290 infinity II.

Method A: Mobile Phase: A: 0.1% HCOOH in H₂O: ACN (95:5), B: ACN; Flow Rate: 1.5 mL/min; Column: ZORBAX XDB C-18 (50×4.6 mm) 3.5 μM.

Method B: Mobile Phase: A: 10 mM NH₄HCO₃ in water, B: ACN; Flow Rate: 1.2 mL/min; Column: XBridge C8 (50×4.6 mm), 3.5 μM.

Method C: Mobile Phase: A: 10 mM Ammonium acetate in water, B: ACN; Flow Rate: 1.2 mL/min; Column: Zorbax Extend C18 (50×4.6 mm), 5 μM.

Method D: Mobile Phase: A: 0.1% TFA in H₂O: ACN (95:5), B: 0.1% TFA in ACN; Flow Rate: 1.5 mL/min; Column: XBridge C8 (50×4.6 mm), 3.5 μM.

### HPLC:

Instrument name: Agilent 1260 Infinity II series instruments as followed using % with UV detection (maxplot).

Method A: Method: A: 10 mM NH₄HCO₃ in water, B: ACN; flow rate: 1.0 mL/min; column: X-Bridge C8 (50×4.6 mm, 3.5 μm).

Method B: Method: A: 0.1% TFA in water, B: 0.1% TFA in ACN; flow rate: 2.0 mL/min; column: X-Bridge C8 (50×4.6 mm, 3.5 μm).

Method C: Method: A: 10 mM Ammonium acetate in milli-q water, B: ACN; flow rate: 1.0 ml/min; column: X-Bridge C8 (50×4.6 mm, 3.5 μm).

Method D: Method: A: 0.1% TFA in water, B: ACN, flow rate: 2.0 mL/min; column: X-Bridge C8 (50×4.6 mm, 3.5 μm).

Method E: Method: A: 0.1% FA in water, B: ACN, flow rate: 2.0 mL/min; column: X-Bridge C8 (50×4.6 mm, 3.5 μm).

### Chiral SFC:

Instrument name: PIC SFC 10 (analytical)

Ratio between CO₂ and co-solvent is ranging between 60:40 and 80:20

Method A: Mobile Phase: 0.5% Isopropyl Amine in Methanol, flow rate: 3 mL/min; column: Lux A1.

Method B: Mobile Phase: 0.5% Isopropyl Amine in IPA, flow rate: 4 mL/min; column: Chiracel OD-H (250×4.6 mm, 5 μm).

Method C: Mobile Phase: IPA, flow rate: 3 mL/min; column: YMC Amylose-SA (250×4.6 mm, 5 μm).

Method D: Mobile Phase: IPA, flow rate: 3 mL/min; column: YMC Amylose-C (250×4.6 mm, 5 μm).

### Prep-HPLC:

Instrument name: Agilent 1290 Infinity II

Method A: Mobile phase: A: water; Mobile phase; B: ACN, Flow: 2.0 mL/min; Column: X-Bridge C18 (50×4.6 mm, 3.5 μM).

### Step 1: methyl 2-bromo-2-(4-chlorophenyl)acetate (SM1)

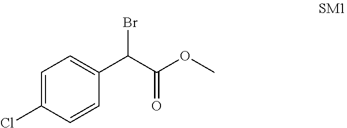

To a stirred solution of methyl 2-(4-chlorophenyl)acetate (100 g, 0.54 mol) in DCE (1 Lit) at 10-15° C., AlBN (8.90 g, 0.05 mol) and followed by NBS (127 g, 0.65 mol) was added portion wise. The resulting reaction mixture was continued to stir 16 h at 65° C. After the completion of the reaction (monitored by TLC), the reaction mixture was poured in ice-cold water (2 Lit). The aqueous part was extracted with DCM (2×500 mL), then the combined organic solution was washed with saturated brine solution (2 Lit) and dried over anhydrous Na₂SO₄. The organic part was concentrated under vacuum and the resulting crude was purified by Isolera column chromatography (eluent: 3-8% EtOAc/PE; silica gel: 230-400 mesh) to afford the title compound. Yield: 89.5% (128 g, colourless liquid). ¹H NMR (400 MHz, CDCl₃): δ 7.51 (dd, J=6.4, 2.0 Hz, 2H), 7.37 (dd, J=2.0, 6.4 Hz, 2H), 5.34 (s, 1H), 3.82 (s, 3H).

### Step 2: methyl 2-(4-chlorophenyl)-2-(3,5-dimethoxyphenoxy)acetate (SM2)

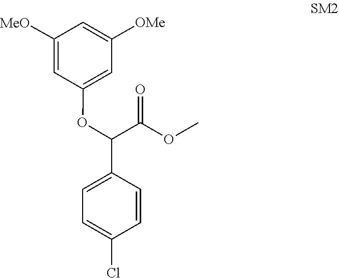 [SMILES: COC(=O)C(Oc1cc(OC)cc(OC)c1)c1ccc(Cl)cc1]

To a stirred solution of methyl 2-bromo-2-(4-chlorophenyl)acetate (SM1) (128 g, 0.48 mol) in acetone (1.3 Lit), K₂CO₃ (111 g, 0.80 mol) and 3,5-dimethoxyphenol (62 g, 0.40 mol) were added at RT. The resulting reaction mixture was stirred 16 h at 70° C. After the completion of the reaction (monitored by TLC), the reaction mixture was filtered through Buchner funnel to remove the excess K₂CO₃ and washed with EtOAc (2×500 mL). The filtrate was concentrated under vacuum, then the obtained residue was dissolved in EtOAc (3×500 mL). The EtOAc layer was washed with dilute HCl (1.5 N, 2×500 mL), water (2 Lit) then brine solution (1 Lit) and dried over anhydrous Na₂SO₄. The organic part was concentrated under vacuum and the resulting crude was purified by Isolera column chromatography (eluent: 10-10% EtOAc/PE; silica gel: 230-400 mesh) to afford the title compound. Yield: 95.5% (129 g, colorless gum). ¹H NMR (400 MHz, CDCl₃): δ 7.53 (d, J=8.4 Hz, 2H), 7.39 (d, J=8.8 Hz, 2H), 6.13 (s, 3H), 5.60 (s, 1H), 3.76 (s, 9H). LCMS: (Method A) 337.1 (M⁺+H), Rt. 2.56 min, 99.08% (Max).

### Step 3: 2-(4-chlorophenyl)-2-(3,5-dimethoxyphenoxy)acetic acid (SM3)

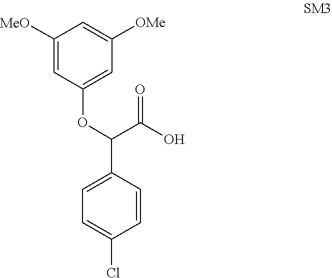 [SMILES: COc1cc(OC)cc(OC(C(=O)O)c2ccc(Cl)cc2)c1]

To a stirred solution of methyl 2-(4-chlorophenyl)-2-(3,5-dimethoxyphenoxy)acetate (SM2) (129 g, 0.38 mol) in MeOH (850 mL) and Water (70 mL), K₂CO₃ (64 g, 0.46 mol) was added and the resulting reaction mixture was stirred 3 h at 50° C. After the completion of reaction (monitored by TLC), the reaction mixture was concentrated under vacuum and the resulting crude was dissolved in water (2 L). The aqueous layer was washed with pet-ether (2×500 mL) and was acidified with dilute HCl (6N, 500 mL). The aqueous layer was extracted with EtOAc (3×500 mL), the EtOAc layer was washed with saturated brine solution (1 L) and then dried over anhydrous Na₂SO₄. The organic part was concentrated under vacuum and the obtained material was forwarded as such without any further purification. Yield: 88.6% (109 g, white solid). ¹H NMR (400 MHz, CDCl₃): δ 7.52 (dd, J=4.0, 2.0 Hz, 2H), 7.40 (dd, J=6.8, 2.0 Hz, 2H), 6.15-6.12 (m, 3H), 5.60 (s, 1H), 3.76 (s, 6H). LCMS: (Method A) 323.1 (M⁺+H), Rt. 2.16 min, 95.72% (Max).

### Step 4: 2-(4-chlorophenyl)-4,6-dimethoxybenzofuran-3(2H)-one (SM4)

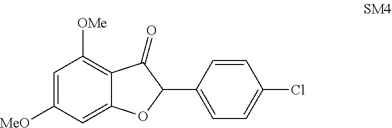 [SMILES: COc1cc(OC)c2c(c1)OC(c1ccc(Cl)cc1)C2=O]

To a stirred solution of 2-(4-chlorophenyl)-2-(3,5-dimethoxyphenoxy)acetic acid (SM3) (25 g, 0.07 mol) in POCl₃ (125 mL), ZnCl₂ (13.7 g, 0.10 mol) was added and the resulting reaction mixture was stirred 16 h at RT. After completion of the reaction (monitored by TLC), the reaction mixture was concentrated under vacuum at 50° C. The resulting crude was poured into ice-cold water (100 mL) and the aqueous layer was extracted with EtOAc (2×300 mL). The EtOAc layer was washed with water (3×500 mL), saturated NaHCO₃ solution (500 mL) and brine solution (500 mL). The organic part was dried over anhydrous Na₂SO₄, concentrated under vacuum and the obtained solid was triturated with methanol (70 mL). The resulting compound in methanol was stirred 15 mins at RT, the obtained solid was filtered off and dried under vacuum at 45° C. to afford the title compound. Yield: 72% (17 g, white solid). ¹H NMR (400 MHz, CDCl₃): δ 7.40-7.35 (m, 4H), 6.28 (d, J=1.6 Hz, 1H), 6.08 (d, J=2.0 Hz, 1H), 5.47 (s, 1H), 3.93 (s, 6H). LCMS: (Method D) 305.0 (M⁺+H), Rt. 2.73 min, 95.86% (Max).

### Step 5: 3-(2-(4-chlorophenyl)-4,6-dimethoxy-3-oxo-2,3-dihydrobenzofuran-2-yl)-3-(3-fluorophenyl)propanal (diastereomeric mixture SM5a, SM5b, SM6a and SM6b)

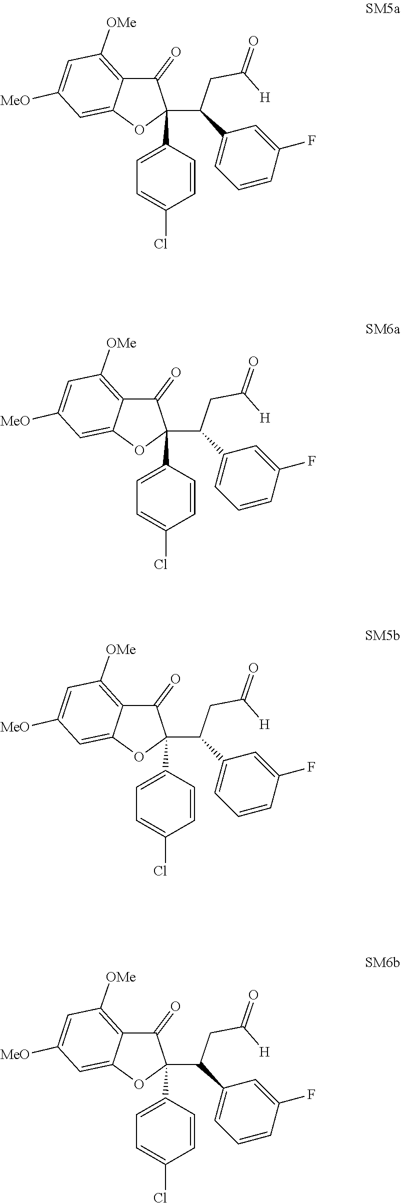

To a stirred solution of 2-(4-chlorophenyl)-4,6-dimethoxybenzofuran-3(2H)-one (SM4) (20 g, 0.06 mol) in butanol (200 mL), (E)-3-(3-fluorophenyl)acrylaldehyde (12.5 g, 0.08 mol), then benzyl trimethylammonium hydroxide (triton B) in MeOH (0.54 g, 0.003 mol) were added at RT and the reaction mixture was stirred 2 h at 65° C. After completion of reaction (monitored by TLC), the reaction mixture was cooled to RT and concentrated under vacuum to remove ^{t}BuOH. The resulting mixture was dissolved in EtOAc (200 mL), then the organic part was washed with water (500 mL) and brine solution (500 mL). The organic layer was separated, dried over anhydrous Na₂SO₄ and concentrated under vacuum. The resulting crude was purified by Isolera column chromatography (eluent: 1:1:0.1% PE/DCM/EtOAc; silica gel: 230-400 mesh) to afford the title compound as diastereomeric mixture (of SM5a, SM5b, SM6a and SM6b).Yield: 26% (8.0 g, pale yellow solid; 2:1 syn & anti diastereomeric mixture). ¹H NMR (400 MHz, CDCl₃): δ 9.45 (s, 1H), 7.71-7.69 (m, 1H), 7.38-7.36 (m, 2H), 7.16-7.12 (m, 2H), 7.07-7.03 (m, 2H), 6.94-6.80 (m, 1H), 4.24-4.20 (m, 1H), 3.92 (s, 3H), 3.85 (s, 3H), 3.11-3.07 (m, 1H), 2.67-2.62 (m, 1H). LCMS: (Method C) 455.0 (M⁺+H), Rt. 3.44 min, 55.43% (Max).

### Step 6: 4-(2-(4-chlorophenyl)-4,6-dimethoxy-3-oxo-2,3-dihydrobenzofuran-2-yl)-4-(3-fluorophenyl)-2-((trimethylsilyl)oxy)butanenitrile (diastereomeric mixture SM7a, SM7b, SM8a and SM8b)

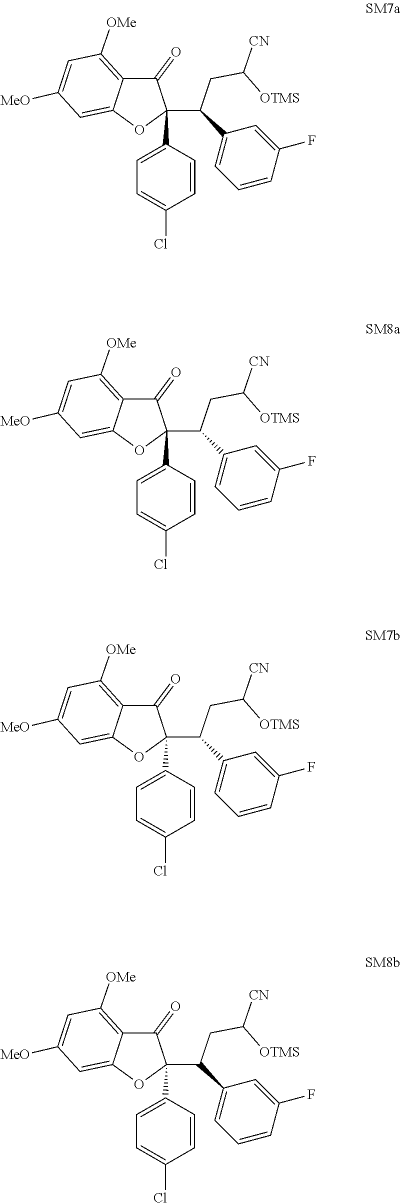

To a stirred solution of (3-(2-(4-chlorophenyl)-4,6-dimethoxy-3-oxo-2,3-dihydrobenzofuran-2-yl)-3-(3-fluorophenyl)propanal (diastereomeric mixture of SM5a, SM5b, SM6a and SM6) (9.5 g, 20.92 mmol) in ACN (100 mL), TMSCN (5.2 mL, 41.85 mmol) and ZnI₂ (0.33 g, 1.04 mmol) were added and the resulting mixture was stirred 16 h at RT. After completion of reaction (monitored by TLC), the reaction mixture was quenched with water (100 mL) and the aqueous layer was extracted with EtOAc (200 mL). The organic layer was separated, washed with saturated brine solution (100 mL), dried over anhydrous Na₂SO₄ and concentrated under vacuum. The resulting crude material was forwarded as such to the next step without any further purification. Yield: 10 g (crude, pale yellow solid); diastereomeric mixture of SM7a, SM7b, SM8a and SM8b. LCMS: (Method C) 454.0 (M⁺+H), Rt. 4.07 min, 68.63% (Max).

### Step 7: 3a-(4-chlorophenyl)-3-(3-fluorophenyl)-8b-hydroxy-6,8-dimethoxy-1-((trimethylsilyl)oxy)-2,3,3a,8b-tetrahydro-1H-cyclopenta[b]benzofuran-1-carbonitrile (diastereomeric mixture of SM9)

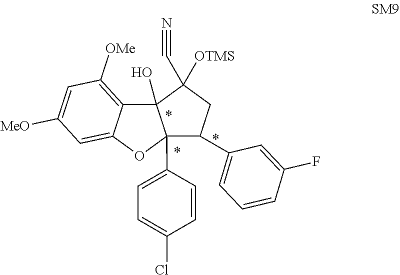

To a stirred solution of 4-(2-(4-chlorophenyl)-4,6-dimethoxy-3-oxo-2,3-dihydrobenzofuran-2-yl)-4-(3-fluorophenyl)-2-((trimethylsilyl)oxy)butanenitrile (diastereomeric mixture of SM7a, SM7b, SM8a and SM8b) (5 g, 9.03 mol) in dry THE (15 mL), LDA (2 M THF, 13.5 mL, 27.07 mol) was added dropwise at −78° C. and the resulting mixture was stirred 2 h at −78° C. After completion of the reaction (monitored by TLC), the reaction mixture was quenched with saturated NH₄Cl solution (50 mL), the aqueous layer was extracted with EtOAc (2×50 mL). The combined organic layer was washed with water (50 mL), brine solution (50 mL) and dried over anhydrous Na₂SO₄. The organic part was concentrated under vacuum and the resulting crude was forwarded as such to the next step without any further purification as diastereomer mixture. Yield: 5 g (crude, pale yellow solid).

### Step 8: 3a-(4-chlorophenyl)-3-(3-fluorophenyl)-8b-hydroxy-6,8-dimethoxy-2,3,3a,8b-tetrahydro-1H-cyclopenta[b]benzofuran-1-one (diastereomeric mixture of SM10; the isomers SM11 and SM12)

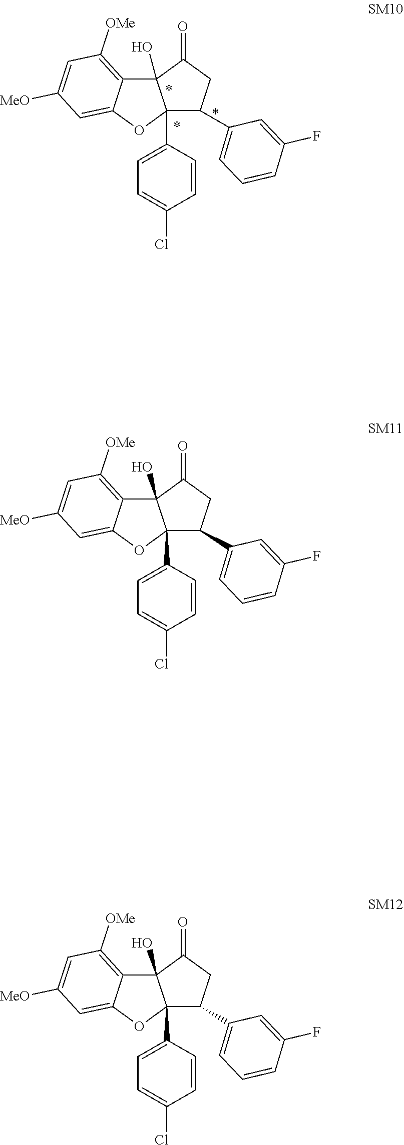 [SMILES: COc1cc(OC)c2c(c1)OC1(c3ccc(Cl)cc3)C(c3cccc(F)c3)CC(=O)C21O ; COc1cc(OC)c2c(c1)O[C@@]1(c3ccc(Cl)cc3)[C@H](c3cccc(F)c3)CC(=O)[C@@]21O ; COc1cc(OC)c2c(c1)O[C@@]1(c3ccc(Cl)cc3)[C@@H](c3cccc(F)c3)CC(=O)[C@@]21O]

To a stirred solution of 3a-(4-chlorophenyl)-3-(3-fluorophenyl)-8b-hydroxy-6,8-dimethoxy-1-((trimethylsilyl)oxy)-2,3,3a,8b-tetrahydro-1H-cyclopenta[b]benzofuran-1-carbonitrile (diastereomer mixture of SM9) (10 g, 18.05 mol) in dry THE (100 mL), TBAF (1M in THF, 22.5 mL, 22.56 mol) was added at 10-15° C. and the resulting mixture was stirred 16 h at RT. After completion of the reaction (monitored by TLC), the reaction mixture was quenched with water (100 mL) and the aqueous layer was extracted with EtOAc (2×100 mL). The combined organic layer was separated, washed with brine solution (100 mL) and dried over anhydrous Na₂SO₄. The organic part was concentrated under vacuum and the resulting crude was purified by Isolera column chromatography (eluent: 1:1:0.1% PE/DCM/EtOAc; silica gel: 230-400 mesh) to afford the title compound SM10 (2.2 g). The isomers were separated by SFC ((Mobile Phase: IPA, flow rate: 3 mL/min; column: Hilic (250×4.6 mm, 5 μm)); the first eluting peak was concentrated under vacuum to afford the title compound SM12. Yield: 20% (1.6 g, off white solid). ¹H NMR (400 MHz, CDCl₃): δ 7.18 (d, J=2.8 Hz, 2H), 7.15 (t, J=2.0 Hz, 1H), 7.02 (dd, J=6.8, 2.0 Hz, 2H), 6.86 (t, J=1.6 Hz, 1H), 6.75 (d, J=7.6 Hz, 2H), 6.37 (d, J=2.0 Hz, 1H), 6.14 (d, J=2.0 Hz, 1H), 3.94 (s, 1H), 3.88 (s, 6H), 3.22 (s, 1H), 3.14-3.06 (m, 1H), 3.02-2.94 (m, 1H). LCMS: (Method C) 455.9 (M⁺+2), Rt. 3.42 min, 97.71% (Max). HPLC: (Method A) Rt. 13.19 min, 98.33% (Max).

The isomers were separated by SFC ((Mobile Phase: IPA, flow rate: 3 mL/min; column: Hilic (250×4.6 mm, 5 μm)); the second eluting peak was concentrated under vacuum to afford the title compound SM11. Yield: 2.5% (250 mg, off white solid). ¹H NMR (400 MHz, CDCl₃): δ 7.17-7.10 (m, 3H), 7.03-7.00 (m, 2H), 6.84-6.74 (m, 3H), 6.37 (d, J=2.0 Hz, 1H), 6.14 (d, J=1.6 Hz, 1H), 3.94-3.91 (m, 1H), 3.88 (s, 3H), 3.85 (s, 3H), 3.24 (s, 1H), 3.09-3.07 (m, 1H), 3.02-2.99 (m, 1H). LCMS: (Method C) 454.9 (M⁺+H), Rt. 3.01 min, 97.92% (Max). HPLC: (Method C) Rt. 6.69 min, 99.64% (Max).

### Step 9: 3a-(4-chlorophenyl)-3-(3-fluorophenyl)-8b-hydroxy-6,8-dimethoxy-2,3,3a,8b-tetrahydro-1H-cyclopenta[b]benzofuran-1-one O-methyl oxime (SM13)

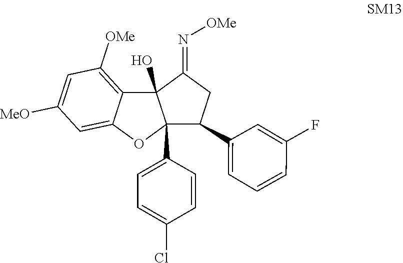 [SMILES: CO/N=C1\C[C@@H](c2cccc(F)c2)[C@]2(c3ccc(Cl)cc3)Oc3cc(OC)cc(OC)c3[C@]12O]

To a stirred solution of 3a-(4-chlorophenyl)-3-(3-fluorophenyl)-8b-hydroxy-6,8-dimethoxy-2,3,3a,8b-tetrahydro-1H-cyclopenta[b]benzofuran-1-one (SM12) (0.9 g, 1.97 mol) in pyridine/ethanol (1:1, 10 mL), O-methyl hydroxylamine hydrochloride (0.87 g, 9.89 mol) was added and the resulting mixture was refluxed 3 h at 70° C. After completion of reaction (monitored by TLC), the reaction mixture was concentrated under vacuum and the obtained residue was dissolved in water (50 mL) and the aqueous layer was extracted with EtOAc (2×20 mL). Combined organic layers were separated, washed with diluted HCl (10 mL), water (20 mL), saturated brine solution (20 mL), dried over anhydrous Na₂SO₄ and concentrated under reduced pressure to get title compound. Yield: 80% (0.77 g, off white solid). ¹H NMR (400 MHz, CDCl₃): δ 7.18-7.17 (m, 2H), 7.11-7.09 (m, 3H), 6.82-6.77 (m, 3H), 6.32 (d, J=2.0 Hz, 1H), 6.15 (d, J=2.0 Hz, 1H), 4.11 (s, 3H), 3.87 (s, 6H), 3.76-3.71 (m, 1H), 3.12 (d, J=4.4 Hz, 1H), 3.08 (d, J=3.2 Hz, 1H), 2.71 (s, 1H). LCMS: (Method C) 466.0 (M⁺−18+H), Rt. 3.65 min, 99.09% (Max). HPLC: (Method C) Rt. 6.99 min, 99.80% (Max).

### Step 10: (1S,3S,3aR,8bS)-1-amino-3a-(4-chlorophenyl)-3-(3-fluorophenyl)-6,8-dimethoxy-1,2,3,3a-tetrahydro-8bH-cyclopenta[b]benzofuran-8b-ol and (1R,3R,3aS,8bR)-1-amino-3a-(4-chlorophenyl)-3-(3-fluorophenyl)-6,8-dimethoxy-1,2,3,3a-tetrahydro-8bH-cyclopenta[b]benzofuran-8b-ol (+/−)(SM14)

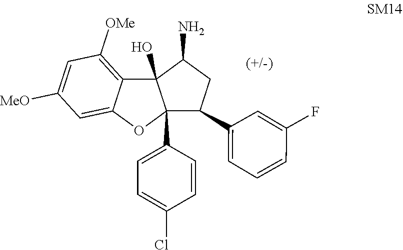 [SMILES: COc1cc(OC)c2c(c1)O[C@@]1(c3ccc(Cl)cc3)[C@H](c3cccc(F)c3)C[C@H](N)[C@@]21O]

To a stirred solution of 3a-(4-chlorophenyl)-3-(3-fluorophenyl)-8b-hydroxy-6,8-dimethoxy-2,3,3a,8b-tetrahydro-1H-cyclopenta[b]benzofuran-1-one O-methyl oxime (SM13) (0.75 g, 1.549 mol) in dry THE (5 mL), BH₃·THF (31 mL, 30.99 mol) was added at 0° C. and the resulting mixture was stirred 16 h at 70° C. After completion of the reaction (monitored by TLC), the reaction mixture was quenched with 10% NaOH solution (20 mL) and stirred 30 min at RT. The aqueous layer was extracted with EtOAc (30 mL), the organic layer was separated and dried over anhydrous Na₂SO₄. The organic part was concentrated under vacuum to afford the crude compound which was forwarded as such to the next step without any further purification. Yield: 700 mg (crude, off white solid). ¹H NMR (400 MHz, CDCl₃): δ 7.22-7.08 (m, 5H), 6.85-6.77 (m, 3H), 6.26 (d, J=1.6 Hz, 1H), 6.11 (d, J=1.2 Hz, 1H), 3.95-3.92 (m, 1H), 3.91 (s, 3H), 3.83 (s, 3H), 3.65-3.60 (m, 1H), 2.57-2.54 (m, 1H), 2.20-2.13 (m, 1H), 1.43 (s, 1H), 1.31-1.26 (m, 2H).

### Step 11: 3a-(4-chlorophenyl)-3-(3-fluorophenyl)-6,8-dimethoxy-1,2,3,3a-tetrahydro-8bH-cyclopenta[b]benzofuran-1,8b-diol (SM15)

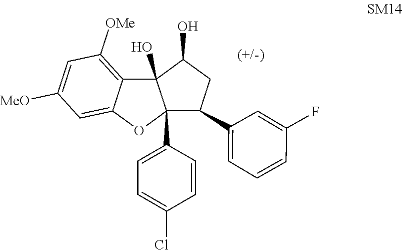 [SMILES: COc1cc(OC)c2c(c1)O[C@@]1(c3ccc(Cl)cc3)[C@H](c3cccc(F)c3)C[C@H](O)[C@@]21O]

To a stirred solution of 3a-(4-chlorophenyl)-3-(3-fluorophenyl)-8b-hydroxy-6,8-dimethoxy-2,3,3a,8b-tetrahydro-1H-cyclopenta[b]benzofuran-1-one (SM12) (0.025 g, 0.05 mmol) in ACN (1 mL) at 0° C., NaBH₄ (41 mg, 1.1 mmol) was added. Then AcOH (0.1 mL) was added and the resulting mixture was stirred 6 h at RT. After completion of the reaction (monitored by TLC), the reaction mixture was quenched with water (10 mL) and aqueous layer was extracted with DCM (2×15 mL). The combined organic layer was separated, dried over anhydrous Na₂SO₄ and was concentrated under vacuum. The resulting crude material was purified by prep-HPLC (Method A) to afford the title compound. Yield: 16% (4.0 mg, off white solid). ¹H NMR (400 MHz, CDCl₃): δ 7.28-7.23 (m, 2H), 7.18-7.15 (m, 2H), 7.09-7.06 (m, 1H), 6.82-6.78 (m, 3H), 6.29 (d, J=2.0 Hz, 1H), 6.14 (d, J=2.0 Hz, 1H), 4.85-4.81 (m, 1H), 3.89 (s, 3H), 3.87 (s, 3H), 3.52-3.46 (m, 1H), 3.09 (d, J=2.80 Hz, 1H), 2.69 (d, J=1.60 Hz, 1H), 2.67-2.62 (m, 1H), 2.40-2.35 (m, 1H). LCMS: (Method A) 439.0 (M⁺−18+H), Rt. 3.52 min, 93.37% (Max). HPLC: (Method B) Rt. 4.86 min, 96.97% (Max).

### Step 12: 3a-(4-chlorophenyl)-3-(3-fluorophenyl)-6,8-dimethoxy-1,2,3,3a-tetrahydro-8bH-cyclopenta[b]benzofuran-1,8b-diol (SM16)

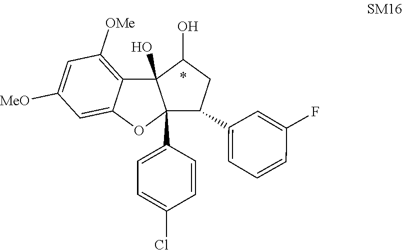 [SMILES: COc1cc(OC)c2c(c1)O[C@@]1(c3ccc(Cl)cc3)[C@@H](c3cccc(F)c3)CC(O)[C@@]21O]

To a stirred solution of 3a-(4-chlorophenyl)-3-(3-fluorophenyl)-8b-hydroxy-6,8-dimethoxy-2,3,3a,8b-tetrahydro-1H-cyclopenta[b]benzofuran-1-one (SM11) (0.05 g, 0.11 mmol) in ACN (3 mL) at 0° C., NaBH₄ (83 mg, 2.19 mmol) was added. Then AcOH (0.2 mL) was added and the resulting mixture was stirred 6 h at RT. After completion of the reaction (monitored by TLC), the reaction mixture was quenched with water (10 mL) and aqueous layer was extracted with DCM (2×30 mL). The organic layer was separated, dried over anhydrous Na₂SO₄ and concentrated under vacuum. The obtained crude material was purified by prep-HPLC (Method A) to afford the title compound. Yield: 12% (6.0 mg, off white solid). ¹H NMR (400 MHz, CDCl₃): δ 7.41-7.37 (m, 4H), 7.18-7.13 (m, 1H), 6.94-6.90 (m, 1H), 6.76-6.70 (m, 2H), 6.18 (d, J=2.0 Hz, 1H), 6.12 (d, J=2.0 Hz, 1H), 4.71-4.67 (m, 1H), 3.97-3.91 (m, 1H), 3.86 (s, 3H), 3.83 (s, 3H), 3.74-3.70 (m, 1H), 2.39-2.33 (m, 1H), 2.15-2.06 (m, 1H). LCMS: (Method A) 457.9 (M⁺+H), Rt. 2.31 min, 98.97% (Max). HPLC: (Method E) Rt. 10.33 min, 98.27% (Max).

### 1-Amino-3a-(4-bromophenyl)-3-(3-fluorophenyl)-6,8-dimethoxy-1,2,3,3a-tetrahydro-8bH-cyclopenta[b]benzofuran-8b-ol (SM17)

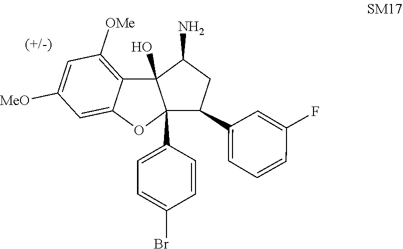 [SMILES: COc1cc(OC)c2c(c1)O[C@@]1(c3ccc(Br)cc3)[C@H](c3cccc(F)c3)C[C@H](N)[C@@]21O]

was prepared analogously to SM14.

### Step 13: Benzyl (3a-(4-bromophenyl)-3-(3-fluorophenyl)-8,8b-dihydroxy-6-methoxy-2,3,3a,8b-tetrahydro-1H-cyclopenta[b]benzofuran-1-yl)carbamate (SM18)

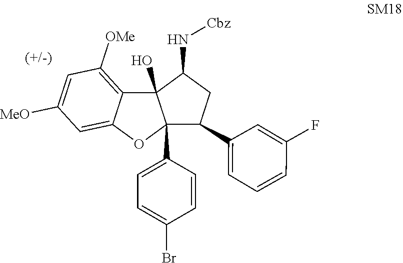 [SMILES: COc1cc(OC)c2c(c1)O[C@@]1(c3ccc(Br)cc3)[C@H](c3cccc(F)c3)C[C@H](NC(=O)OCc3ccccc3)[C@@]21O]

To a stirred solution of 1-amino-3a-(4-bromophenyl)-3-(3-fluorophenyl)-6,8-dimethoxy-1,2,3,3a-tetrahydro-8bH-cyclopenta[b]benzofuran-8b-ol (900 mg, 1.79 mmol)) in 1, 4-dioxane:water (22 mL, 10:1), sodium bicarbonate (453 mg, 5.40 mmol) and then benzyl carbonochloridate (921 mg, 2.70 mmol) were added at 0° C. The reaction mixture stirred 16 h at RT. After completion of the reaction (monitored by TLC), the reaction mixture was diluted with water (15 mL) and the aqueous layer was extracted with EtOAc (2×100 mL). The combined organic layer was washed with brine (50 mL), dried over anhydrous Na₂SO₄, filtered and concentrated under vacuum. The resulting crude material was triturated with n-hexane, the obtained solid was collected by filtration and dried under vacuum to afford the title compound. Yield: 82% (950 mg, off white solid). LCMS: (Method D) 617 (M⁺−18), Rt. 2.69 min, 98.91% (Max).

### Step 14: Benzyl (3a-(4-cyanophenyl)-3-(3-fluorophenyl)-8b-hydroxy-6,8-dimethoxy-2,3,3a,8b-tetrahydro-1H-cyclopenta[b]benzofuran-1-yl)carbamate (SM19)

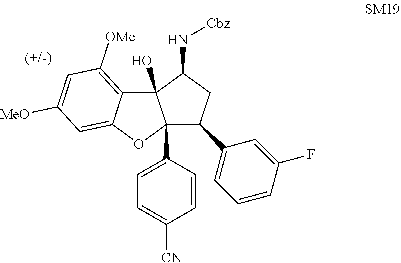 [SMILES: COc1cc(OC)c2c(c1)O[C@@]1(c3ccc(C#N)cc3)[C@H](c3cccc(F)c3)C[C@H](NC(=O)OCc3ccccc3)[C@@]21O]

To a stirred solution of benzyl (3a-(4-bromophenyl)-3-(3-fluorophenyl)-8,8b-dihydroxy-6-methoxy-2,3,3a,8b-tetrahydro-1H-cyclopenta[b]benzofuran-1-yl)carbamate (250 mg, 0.39 mmol) in DMF (3 mL), dicyanozinc (231 mg, 1.97 mmol) was added and nitrogen gas was purged thoroughly for 8-10 min. To this reaction mixture, xantphos (45.6 mg, 0.08 mmol) and Pd₂(dba)₃ (36.1 mg, 0.04 mmol) were added under nitrogen atmosphere and the reaction was continued 3 h at 120° C. After completion of the reaction (monitored by TLC), the reaction mixture was filtered through celite pad, then washed with EtOAc (100 mL). The filtrate was washed with water (2×50 mL), brine (50 mL) and was dried over anhydrous Na₂SO₄. The organic part was filtered, concentrated under vacuum and the resulting crude material was purified by Biotage Isolera column chromatography (eluent: 30-40% EtOAc/PE; silica gel: 230-400 mesh) to afford the title compound. Yield: 52.1% (120 mg, off white solid). ¹H NMR (400 MHz, CDCl₃): δ 7.54-7.38 (m, 7H), 7.32-7.30 (m, 2H), 7.10-7.02 (m, 1H), 6.82-6.77 (m, 3H), 6.26 (d, J=2.0 Hz, 1H), 6.07 (d, J=1.6 Hz, 1H), 5.88 (bs, 1H), 5.25-5.17 (m, 2H), 4.63-4.56 (m, 1H), 3.85 (s, 3H), 3.72-3.70 (m, 4H), 2.76-2.75 (m, 1H), 2.37-2.31 (m, 1H), 2.12-2.07 (m, 1H). LCMS: (Method D) 563.3 (M⁺−18+H), Rt. 2.54 min, 99.25% (Max).

### Step 15: 4-(1-Amino-3-(3-fluorophenyl)-8b-hydroxy-6,8-dimethoxy-1,2,3,8b-tetrahydro-3aH-cyclopenta[b]benzofuran-3a-yl)benzonitrile (SM20)

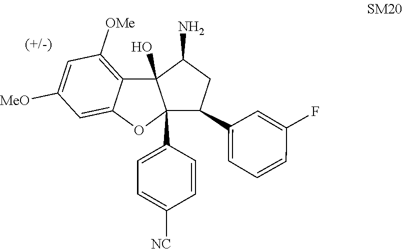 [SMILES: COc1cc(OC)c2c(c1)O[C@@]1(c3ccc(C#N)cc3)[C@H](c3cccc(F)c3)C[C@H](N)[C@@]21O]

To a stirred solution of benzyl (3a-(4-cyanophenyl)-3-(3-fluorophenyl)-8b-hydroxy-6,8-dimethoxy-2,3,3a,8b-tetrahydro-1H-cyclopenta[b]benzofuran-1-yl)carbamate (480 mg, 0.83 mmol) in MeOH (10 mL), nitrogen gas was thoroughly purged or a period of 5 min. To this reaction mixture, 5% Pd/C (210 mg, 0.09 mmol) was added under nitrogen atmosphere. The resulting reaction mixture was stirred under hydrogen gas (Balloon) pressure 2 h at room temperature. After completion of the reaction (monitored by TLC), the reaction mixture was filtered through celite pad, was washed with MeOH (100 mL). The filtrate was concentrated under reduced pressure and the resulting crude material was triturated with n-hexane. The obtained solid was collected by filtration and dried under vacuum to afford the title compound. Yield: 85% (345 mg, off white solid). ¹H NMR (400 MHz, CDCl₃): δ 7.44-7.42 (m, 2H), 7.34-7.32 (m, 2H), 7.11-7.05 (m, 1H), 6.81-6.77 (m, 3H), 6.27 (d, J=1.6 Hz, 1H), 6.12 (d, J=2.0 Hz, 1H), 3.97-3.94 (m, 1H), 3.88 (s, 3H), 3.85 (s, 3H), 3.71-3.66 (m, 1H), 2.60-2.54 (m, 1H), 2.18-2.09 (m, 1H). LCMS: (Method B) 445.1 (M⁺−H), Rt. 2.81 min, 90.88% (Max).

### Example 1: 3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-4-methylpiperazine-1-carboxamide (Syn-racemic mixture of N-((1S,3S,3aR,8bS)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-4-methylpiperazine-1-carboxamide and N-((1R,3R,3aS,8bR)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-4-methylpiperazine-1-carboxamide (2 and 3) enantiomers; 1)

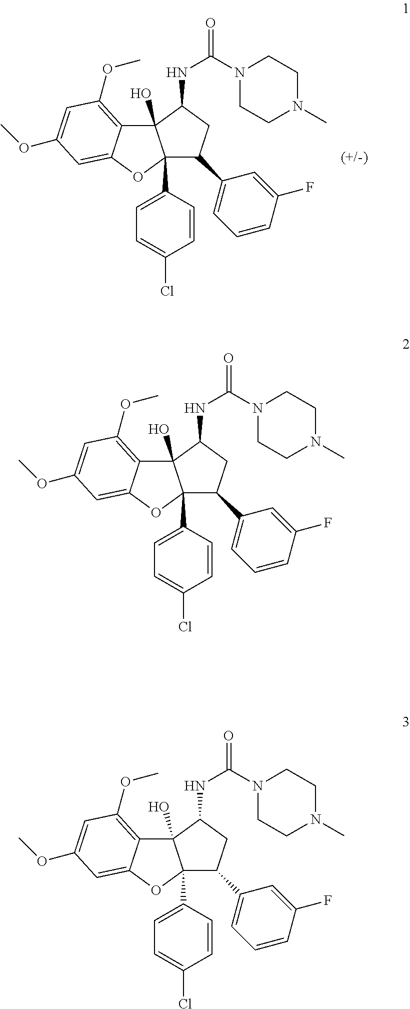

To a stirred solution of SM14 (0.1 g, 0.22 mmol) in dry DCM (5 mL) at 0° C., DIPEA (56.5 mg, 0.44 mmol) was added dropwise. Then 4-methyl-1-piperazinecarbonyl chloride hydrochloride (43.6 mg, 0.22 mmol) was added at 0° C. and the resulting mixture was stirred 16 h at RT. After the completion of reaction (monitored by TLC), the reaction mixture was quenched with water (10 mL) and the aqueous layer was extracted with DCM (2×25 mL). The combined organic layer was washed with water (20 mL), brine solution (20 mL) and dried over anhydrous Na₂SO₄. The organic part was concentrated under vacuum and the resulting crude was purified by Isolera column chromatography (eluent: 5-10% DCM/MeOH; silica gel: 230-400 mesh) to afford the title compound as Syn racemic (1). Yield: 13% (55 mg, off white solid). ¹H NMR (400 MHz, CDCl₃): δ 7.13-7.06 (m, 5H), 6.90-6.78 (m, 3H), 6.25 (d, J=2.0 Hz, 1H), 6.07 (d, J=2.0 Hz, 1H), 5.83 (d, J=5.6 Hz, 1H), 4.62-4.58 (m, 1H), 3.85 (s, 3H), 3.78 (s, 3H), 3.75-3.72 (m, 1H), 3.52-3.50 (m, 4H), 2.88-2.82 (m, 1H), 2.49-2.47 (m, 4H), 2.38-2.36 (m, 3H), 2.34-2.31 (m, 1H). LCMS: (Method A) 580.2 (M⁺−H), Rt. 1.63 min, 99.79% (Max). HPLC: (Method B) Rt. 3.69 min, 98.48% (Max).

### Examples 2 and 3: N-((1S,3S,3aR,8bS)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-4-methylpiperazine-1-carboxamide and N-((1R,3R,3aS,8bR)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-4-methylpiperazine-1-carboxamide (2 and 3)

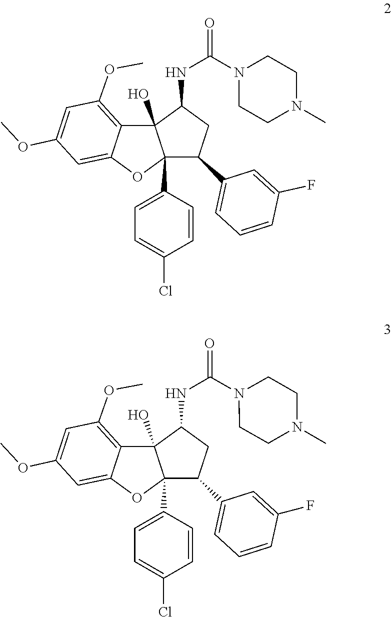

The enantiomers of syn-racemic compound 1 (50 mg, 0.08 mmol) was separated by SFC (Method B). The first eluting peak was concentrated under vacuum at 40° C. to afford the title compound 2 of chiral purity 100% and the second eluting peak afforded the title compound 3 of purity 99.49%.

Analytical data of 2: Yield: 22% (11 mg, white solid). ¹H NMR (400 MHz, DMSO-d₆): δ 7.17-7.12 (m, 5H), 6.87-6.84 (m, 2H), 6.76 (d, J=10.4 Hz, 1H), 6.33 (d, J=1.6 Hz, 1H), 6.24 (d, J=5.6 Hz, 1H), 6.14 (d, J=2.0 Hz, 1H), 5.66 (s, 1H), 4.39-4.36 (m, 1H), 3.78 (s, 3H), 3.69 (s, 3H), 3.57-3.52 (m, 1H), 3.42-3.31 (m, 4H), 2.68-2.67 (m, 4H), 2.53-2.47 (m, 3H), 2.43-2.40 (m, 1H), 2.34-2.31 (m, 1H). LCMS: (Method B) 580.2 (M⁺−H), Rt. 2.41 min, 97.06% (Max). HPLC: (Method C) Rt. 4.39 min, 99.92% (Max). Chiral SFC: (Method B) Rt. 1.89 min, 99.39% (Max).

Analytical data of 3: Yield: 28% (14 mg, white solid). ¹H NMR (400 MHz, DMSO-d₆) δ 7.20-7.12 (m, 5H), 6.89-6.84 (m, 2H), 6.76 (d, J=10.8 Hz, 1H), 6.33 (d, J=2.0 Hz, 1H), 6.23 (d, J=5.6 Hz, 1H), 6.14 (d, J=2.0 Hz, 1H), 5.67 (s, 1H), 4.39-4.36 (m, 1H), 3.78 (s, 3H), 3.69 (s, 3H), 3.57-3.57 (m, 1H), 3.45-3.31 (m, 4H), 2.62-2.58 (m, 4H), 2.53-2.45 (m, 3H), 2.34-2.31 (m, 1H), 2.30-2.26 (m, 1H). LCMS: (Method A) 580.2 (M⁺−H), Rt. 2.81 min, 99.37% (Max). HPLC: (Method C) Rt. 6.87 min, 98.68% (Max). Chiral Purity: (Method B) Rt. 3.09 min, 99.49% (Max).

### Example 4: 3a-(4-chlorophenyl)-3-(3-fluorophenyl)-8b-hydroxy-6,8-dimethoxy-2,3,3a,8b-tetrahydro-1H-cyclopenta[b]benzofuran-1-yl)pyrrolidine-1-carboxamide (Syn racemic; 4)

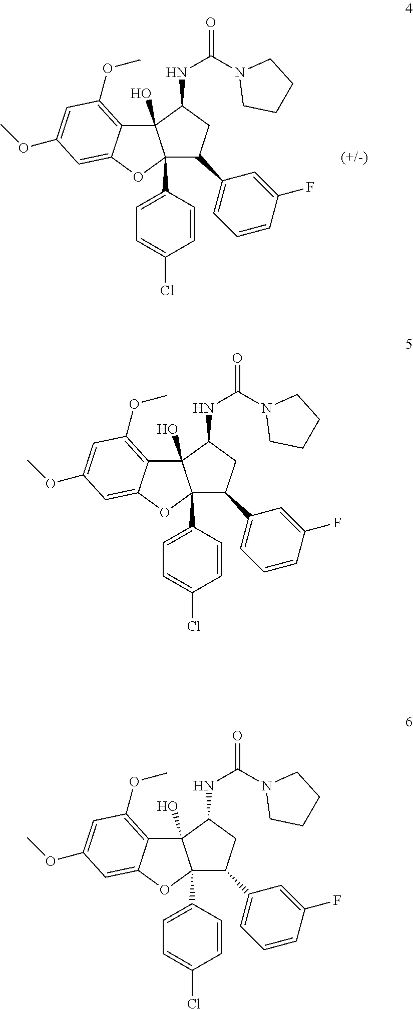

To a stirred solution of SM14 (0.2 g, 0.44 mmol) in dry DCM (5 mL) at 0° C., DIPEA (113.5 mg, 0.88 mmol) was added dropwise. Then pyrrolidine-1-carbonyl chloride (64.5 mg, 0.48 mmol) was added at 0° C. and the resulting mixture was stirred 16 h at RT. After completion of the reaction (monitored by TLC), the reaction mixture was quenched with water (25 mL) and the aqueous layer was extracted with DCM (2×50 mL). The combined organic layer was washed with water (20 mL), brine solution (20 mL) and dried over anhydrous Na₂SO₄. The organic part was concentrated under vacuum and the resulting crude was purified by Isolera column chromatography (eluent: 5-10% DCM/MeOH; silica gel: 230-400 mesh) to afford syn racemic (4) the title compound. Yield: 17% (46 mg, off white solid). ¹H NMR (400 MHz, DMSO-d₆): δ 7.18-7.12 (m, 5H), 6.89-6.85 (m, 2H), 6.79-6.76 (m, 1H), 6.33 (d, J=1.6 Hz, 1H), 6.14 (d, J=1.6 Hz, 1H), 5.84 (d, J=6.0 Hz, 1H), 5.74 (s, 1H), 4.38-4.33 (m, 1H), 3.78 (s, 3H), 3.67 (s, 3H), 3.58-3.53 (m, 1H), 3.33-3.29 (m, 4H), 2.44-2.41 (m, 1H), 2.34-2.27 (m, 1H), 1.89 (s, 4H). LCMS: (Method A) 551.2 (M⁺−H), Rt. 2.49 min, 99.40% (Max). HPLC: (Method B) Rt. 5.45 min, 99.41% (Max).

### Example 5 and 6: N-((1S,3S,3aR,8bS)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)pyrrolidine-1-carboxamide and N-((1R,3R,3aS,8bR)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)pyrrolidine-1-carboxamide (5 and 6)

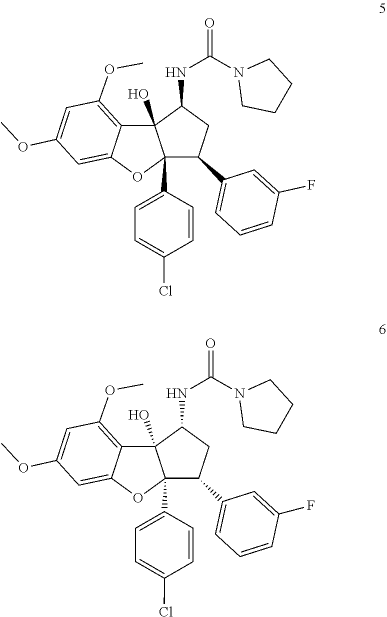 [SMILES: COc1cc(OC)c2c(c1)O[C@@]1(c3ccc(Cl)cc3)[C@H](c3cccc(F)c3)C[C@H](NC(=O)N3CCCC3)[C@@]21O ; COc1cc(OC)c2c(c1)O[C@]1(c3ccc(Cl)cc3)[C@@H](c3cccc(F)c3)C[C@@H](NC(=O)N3CCCC3)[C@]21O]

The enantiomers of syn-racemic compound 4 (45 mg, 0.08 mmol, 4) was separated by SFC (Method C). The first eluting peak was concentrated under vacuum at 40° C. to afford the title compound 5 of chiral purity 100% and the second eluting peak afforded the title compound 6 of purity 99.26%.

Analytical data of 5: Yield: 40% (18 mg, white solid). ¹H NMR (400 MHz, CDCl₃): δ 7.14-7.07 (m, 5H), 6.90-6.84 (m, 2H), 6.80-6.77 (m, 1H), 6.25 (d, J=2.0 Hz, 1H), 6.07 (d, J=2.0 Hz, 1H), 5.52 (d, J=6.4 Hz, 1H), 4.65-4.62 (m, 1H), 3.85 (s, 3H), 3.77-3.72 (m, 4H), 3.48-3.45 (m, 4H), 2.85-2.79 (m, 1H), 2.43-2.34 (m, 1H), 2.01-1.60 (m, 4H). LCMS: (Method A) 553.1 (M⁺+H), Rt. 2.58 min, 98.81% (Max). HPLC: (Method C) Rt. 6.71 min, 99.73% (Max). Chiral SFC: (Method C) Rt. 2.36 min, 98.45% (Max).

Analytical data of 6: Yield: 33% (15 mg, white solid). ¹H NMR (400 MHz, CDCl₃): δ 7.16-7.08 (m, 5H), 6.90-6.84 (m, 2H), 6.80-6.75 (m, 1H), 6.25 (d, J=2.0 Hz, 1H), 6.07 (d, J=2.0 Hz, 1H), 5.51-5.50 (m, 1H), 4.65-4.62 (m, 1H), 3.85 (s, 3H), 3.77-3.72 (m, 4H), 3.48-3.45 (m, 4H), 2.83-2.80 (m, 1H), 2.41-2.37 (m, 1H), 2.01-1.97 (m, 4H). LCMS: (Method A) 551.1 (M⁺−H), Rt. 2.58 min, 99.73% (Max). HPLC: (Method C) Rt. 6.71 min, 99.94% (Max). Chiral Purity: (Method C) Rt. 3.46 min, 99.26% (Max).

### Example 7: 3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-3-isopropylurea (Syn racemic; 7)

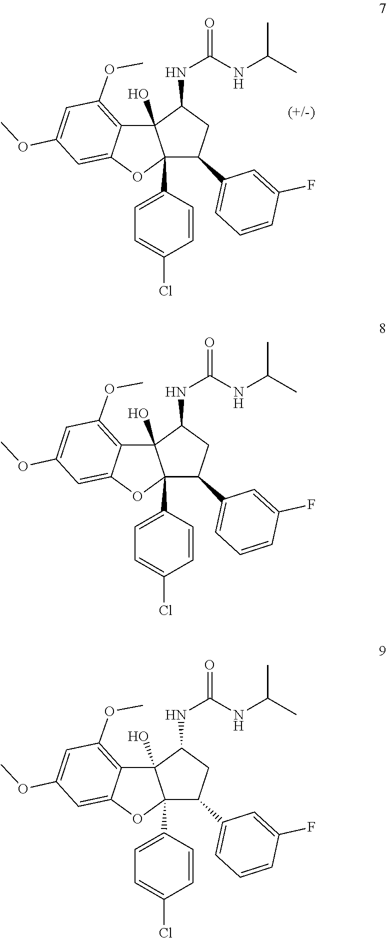 [SMILES: COc1cc(OC)c2c(c1)O[C@@]1(c3ccc(Cl)cc3)[C@H](c3cccc(F)c3)C[C@H](NC(=O)NC(C)C)[C@@]21O ; COc1cc(OC)c2c(c1)O[C@@]1(c3ccc(Cl)cc3)[C@H](c3cccc(F)c3)C[C@H](NC(=O)NC(C)C)[C@@]21O ; COc1cc(OC)c2c(c1)O[C@]1(c3ccc(Cl)cc3)[C@@H](c3cccc(F)c3)C[C@@H](NC(=O)NC(C)C)[C@]21O]

To a stirred solution of SM14 (0.2 g, 0.44 mmol) at 0° C. in dry DCM (5 mL), DIPEA (113.4 mg, 0.88 mmol) was added dropwise and stirred 15 min. Then isopropyl isocyanate (41.01 mg, 0.48 mmol) was added and the resulting reaction mixture was stirred 16 h at RT. After completion of the reaction (monitored by TLC), the reaction mixture was quenched with water (25 mL) and the aqueous part was extracted with DCM (2×50 mL). The combined organic layer was separated, washed with water (20 mL) and brine solution (20 mL). The organic part was dried over anhydrous Na₂SO₄ and concentrated under vacuum to afford the crude material. The resulting crude material was purified by Isolera column chromatography (Eluent: 5-10% DCM/MeOH; silica gel: 230-400 mesh) to afford the title compound as Syn racemic. Yield: 20% (40 mg, off white solid). ¹H NMR (400 MHz, DMSO-d₆): δ 7.19-7.14 (m, 5H), 6.85 (t, J=6.4 Hz, 1H), 6.81 (d, J=7.6 Hz, 1H), 6.73 (d, J=10.4 Hz, 1H), 6.40 (d, J=7.6 Hz, 1H), 6.33 (d, J=2.0 Hz, 1H), 6.15 (d, J=2.0 Hz, 1H), 6.05 (d, J=6.4 Hz, 1H), 5.61 (s, 1H), 4.41-4.35 (m, 1H), 3.79 (s, 3H), 3.74-3.70 (m, 4H), 3.37-3.34 (m, 1H), 2.43-2.38 (m, 1H), 2.18-2.11 (m, 1H), 1.11-1.08 (m, 6H). LCMS: (Method A) 541.2 (M⁺+H), Rt. 2.48 min, 99.78% (Max). HPLC: (Method B) Rt. 6.70 min, 98.81% (Max).

### Example 8 and 9: 1-((1S,3S,3aR,8bS)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-3-methylurea and 1-((1R,3R,3aS,8bR)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-3-isopropylurea (8 and 9)

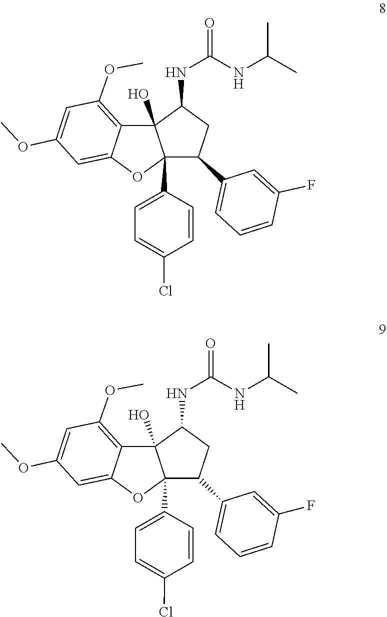

The enantiomers of syn-racemic compound 7—(40 mg, 0.07 mmol, 7) was separated by SFC (method C). The first eluting peak was concentrated under vacuum at 40° C. to afford the title compound 8 of chiral purity 100% and the second eluting peak afforded the title compound 9 of purity 99.39%.

Analytical data of 8: Yield: 25% (10 mg, white solid). ¹H NMR (400 MHz, DMSO-d₆): δ 7.20-7.11 (m, 5H), 6.86 (t, J=6.8 Hz, 1H), 6.81 (d, J=7.6 Hz, 1H), 6.73 (d, J=10.0 Hz, 1H), 6.40 (d, J=7.6 Hz, 1H), 6.33 (d, J=1.6 Hz, 1H), 6.15 (d, J=1.6 Hz, 1H), 6.05 (d, J=6.0 Hz, 1H), 5.62 (s, 1H), 4.41-4.35 (m, 1H), 3.79 (s, 3H), 3.75-3.72 (m, 1H), 3.70 (s, 3H), 3.46-3.41 (m, 1H), 2.42-2.34 (m, 1H), 2.18-2.11 (m, 1H), 1.09 (d, J=2.0 Hz, 6H). LCMS: (Method A) 540.1 (M⁺), Rt. 2.95 min, 99.97% (Max). HPLC: (Method C) Rt. 6.71 min, 98.22% (Max). Chiral Purity: (Method C) Rt. 2.78 min, 100% (Max).

Analytical data of 9: Yield: 25% (10 mg, white solid). ¹H NMR (400 MHz, DMSO-d₆): δ 7.20-7.11 (m, 5H), 6.86 (t, J=6.8 Hz, 1H), 6.81 (d, J=7.6 Hz, 1H), 6.73 (d, J=10.0 Hz, 1H), 6.40 (d, J=7.6 Hz, 1H), 6.33 (d, J=1.6 Hz, 1H), 6.15 (d, J=1.6 Hz, 1H), 6.05 (d, J=6.0 Hz, 1H), 5.62 (s, 1H), 4.41-4.35 (m, 1H), 3.79 (s, 3H), 3.75-3.72 (m, 1H), 3.70 (s, 3H), 3.46-3.41 (m, 1H), 2.42-2.34 (m, 1H), 2.18-2.11 (m, 1H), 1.09 (d, J=2.0 Hz, 6H). LCMS: (Method A) 540.2 (M⁺), Rt. 2.52 min, 99.98% (Max). HPLC: (Method C) Rt. 6.71 min, 96.49% (Max). Chiral Purity: (Method C) Rt. 3.65 min, 99.39% (Max).

### Example 10: 3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-1,1-diethylurea (Syn racemic; 10)

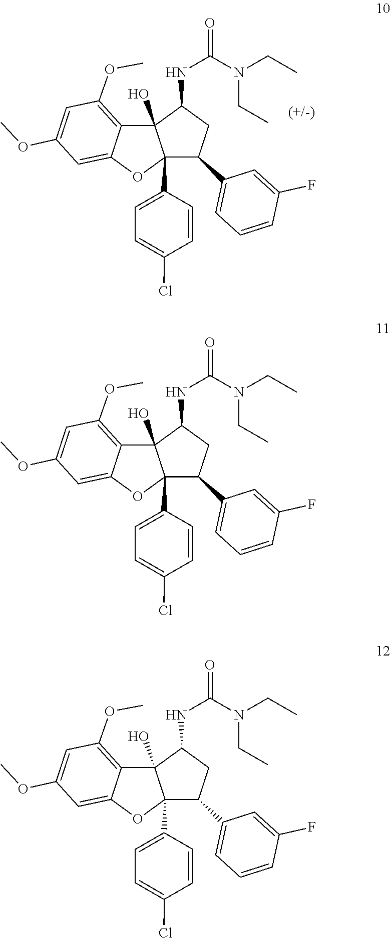 [SMILES: CCN(CC)C(=O)N[C@H]1C[C@@H](c2cccc(F)c2)[C@]2(c3ccc(Cl)cc3)Oc3cc(OC)cc(OC)c3[C@]12O ; CCN(CC)C(=O)N[C@H]1C[C@@H](c2cccc(F)c2)[C@]2(c3ccc(Cl)cc3)Oc3cc(OC)cc(OC)c3[C@]12O ; CCN(CC)C(=O)N[C@@H]1C[C@H](c2cccc(F)c2)[C@@]2(c3ccc(Cl)cc3)Oc3cc(OC)cc(OC)c3[C@@]12O]

To a stirred solution of SM14 (100 mg, 0.01 mmol) in dry DCM (4 mL) at 0-5° C., DIPEA (0.08 mL, 0.04 mmol) was added dropwise and stirred 15 min. Then N, N-diethylcarbamic chloride (0.03 mL, 0.02 mmol) was added and the resulting reaction mixture was stirred 24 h at RT. After completion of the reaction (monitored by TLC), the reaction mixture was concentrated under vacuum and the obtained residue was partitioned in DCM (5 mL) and water (5 mL). The aqueous layer was extracted with DCM (2×15 mL) and the combined organic layer was washed with saturated NaHCO₃ solution (20 mL), water (20 mL) and brine solution (20 mL). The organic part was dried over anhydrous Na₂SO₄ and concentrated under vacuum. The obtained crude material was purified by Prep-HPLC (method A) afford the crude compound as Syn racemic. Yield: 41% (50 mg, colorless liquid). ¹H NMR (400 MHz, CDCl₃): δ 7.18-7.09 (m, 5H), 6.90-6.85 (m, 2H), 6.79 (t, J=6.0 Hz, 1H), 6.25 (d, J=2.0 Hz, 1H), 6.07 (d, J=2.0 Hz, 1H), 5.70 (d, J=6.0 Hz, 1H), 4.67-4.64 (m, 1H), 3.85 (s, 3H), 3.76 (s, 3H), 3.74-3.70 (m, 1H), 3.43-3.37 (m, 4H), 2.85-2.82 (m, 1H), 2.36-2.30 (m, 1H), 1.94 (s, 1H), 1.28-1.24 (m, 6H). LCMS: (Method A) 553.2 (M⁺−H), Rt. 3.10 min, 99.05% (Max). HPLC: (Method B) Rt. 5.53 min, 99.47% (Max).

### Example 11 and 12: 3-((1S,3S,3aR,8bS)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-1,1-diethylurea and 3-((1R,3R,3aS,8bR)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-1,1-diethylurea (11 and 12)

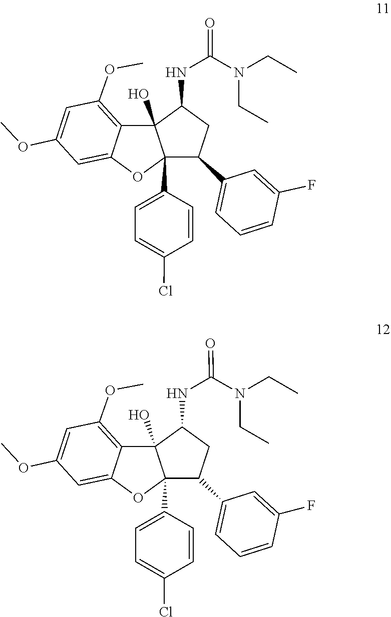

The enantiomers of syn-racemic compound 10 (40 mg; 10) was separated by SFC (Method A). The first eluting peak was concentrated under vacuum at 40° C. to afford the title compound 11 of chiral purity of 98.89% and the second eluting peak afforded the title compound 12 of chiral purity 99.26%.

Analytical data of 11: Yield: 25% (10 mg, white solid). ¹H NMR (400 MHz, CDCl₃): δ 7.18-7.10 (m, 5H), 6.90-6.84 (m, 2H), 6.79 (t, J=6.0 Hz, 1H), 6.25 (d, J=2.0 Hz, 1H), 6.07 (d, J=2.0 Hz, 1H), 5.83 (bs, 1H), 4.67-4.65 (m, 1H), 3.85 (s, 3H), 3.77 (s, 3H), 3.74-3.70 (m, 1H), 3.45-3.36 (m, 4H), 2.85-2.82 (m, 1H), 2.40-2.33 (m, 1H), 1.28-1.25 (m, 6H). LCMS: (Method B) 553.2 (M⁺−H), Rt. 2.61 min, 99.86% (Max). HPLC: (Method B) Rt. 5.69 min, 99.94% (Max). Chiral Purity: (Method A) Rt. 1.74 min, 98.89% (Max).

Analytical data of 12: Yield: 25% (11 mg, white solid). ¹H NMR (400 MHz, CDCl₃): δ 7.14-7.06 (m, 5H), 6.90-6.84 (m, 2H), 6.79 (t, J=6.0 Hz, 1H), 6.25 (d, J=2.0 Hz, 1H), 6.07 (d, J=1.6 Hz, 1H), 5.89 (s, 1H), 4.69-4.63 (m, 1H), 3.85 (s, 3H), 3.75 (s, 3H), 3.74-3.70 (m, 1H), 3.43-3.34 (m, 4H), 2.86-2.80 (m, 1H), 2.40-2.31 (m, 1H), 1.31-1.25 (m, 6H). LCMS: (Method B) 553.2 (M⁺−H), Rt. 2.26 min, 99.37% (Max). HPLC: (Method B) Rt. 5.60 min, 99.94% (Max). Chiral Purity: (Method A) Rt. 3.61 min, 99.26% (Max).

### Example 13: 3a-(4-chlorophenyl)-3-(3-fluorophenyl)-8b-hydroxy-6,8-dimethoxy-2,3,3a,8b-tetrahydro-1H-cyclopenta[b]benzofuran-1-yl)acetamide (Syn racemic; 13)

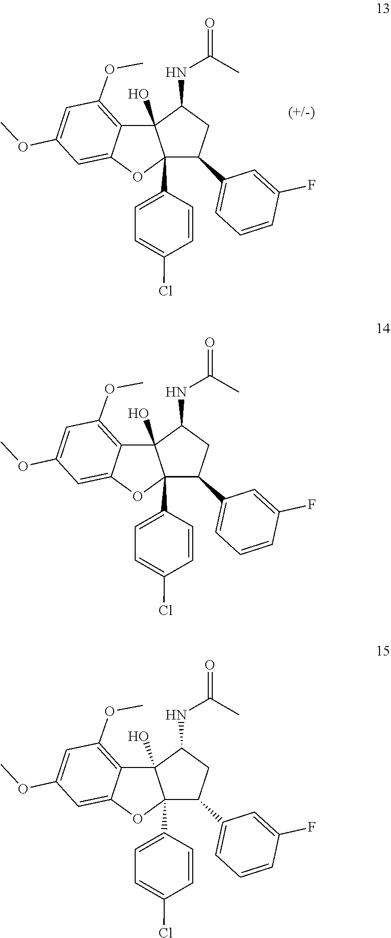 [SMILES: COc1cc(OC)c2c(c1)O[C@@]1(c3ccc(Cl)cc3)[C@H](c3cccc(F)c3)C[C@H](NC(C)=O)[C@@]21O ; COc1cc(OC)c2c(c1)O[C@@]1(c3ccc(Cl)cc3)[C@H](c3cccc(F)c3)C[C@H](NC(C)=O)[C@@]21O ; COc1cc(OC)c2c(c1)O[C@]1(c3ccc(Cl)cc3)[C@@H](c3cccc(F)c3)C[C@@H](NC(C)=O)[C@]21O]

To a stirred solution of SM14 (0.075 g, 0.164 mmol) in dry pyridine (1.5 mL) at 0° C., DMAP (4 mg, 0.03 mmol) was added and stirred 10 min. Then acetic anhydride (25 mg, 0.25 mmol) was added at 0° C. and the resulting mixture was stirred 1 h at RT. After completion of the reaction (monitored by TLC), the reaction mixture was concentrated under vacuum and the resulting crude was purified by Isolera column chromatography (eluent: 30-40% EtOAc/pet ether; silica gel: 230-400 mesh) to afford the title compound as Syn Racemic. Yield: 84% (68 mg, off white solid). ¹H NMR (400 MHz, CDCl₃): δ 7.14-7.07 (m, 5H), 6.88-6.77 (m, 3H), 6.67 (d, J=6.8 Hz, 1H), 6.25 (d, J=2.0 Hz, 1H), 6.08 (d, J=2.0 Hz, 1H), 4.75-4.69 (m, 1H), 3.86 (s, 3H), 3.80 (s, 3H), 3.74-3.69 (m, 1H), 3.51 (m, 1H), 2.87-2.81 (m, 1H), 2.31-2.22 (m, 1H), 2.14 (s, 3H). LCMS: (Method A) 496.1 (M⁺−H), Rt. 2.34 min, 99.69% (Max). HPLC: (Method E) Rt. 5.16 min, 98.68% (Max).

### Example 14 and 15: N-((1S,3S,3aR,8bS)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)acetamide and N-((1R,3R,3aS,8bR)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)acetamide (14 and 15)

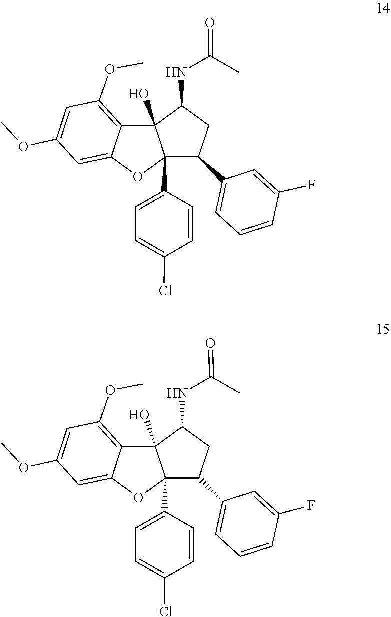

The enantiomers of syn-racemic compound 13—(60 mg; 13)—was separated by SFC (Method C). The first eluting peak was concentrated under vacuum at 40° C. to afford the title compound 14 chiral purity of 99.55% and the second eluting peak afforded the title compound 15 chiral purity 99.12%.

Analytical data of 14: Yield: 11.6% (7 mg, off white solid). ¹H NMR (400 MHz, CDCl₃): δ 7.14-7.07 (m, 5H), 6.88-6.76 (m, 3H), 6.74 (d, J=6.8 Hz, 1H), 6.25 (d, J=1.6 Hz, 1H), 6.09 (d, J=2.0 Hz, 1H), 4.75-4.69 (m, 1H), 3.86 (s, 3H), 3.80 (s, 3H), 3.74-3.69 (m, 1H), 2.87-2.81 (m, 1H), 2.32-2.23 (m, 1H), 2.16 (s, 3H). LCMS: (Method A) 496.1 (M⁺−H), Rt. 2.83 min, 99.69% (Max). HPLC: (Method E) Rt. 5.40 min, 99.80% (Max). Chiral HPLC: (Method C) Rt. 1.83 min, 99.55% (Max).

Analytical data of 15: Yield: 18.3% (11 mg, off white solid). ¹H NMR (400 MHz, CDCl₃): δ 7.14-7.07 (m, 5H), 6.88-6.77 (m, 3H), 6.66 (d, J=6.4 Hz, 1H), 6.25 (s, 1H), 6.09 (s, 1H), 4.74-4.69 (m, 1H), 3.86 (s, 3H), 3.80 (s, 3H), 3.74-3.69 (m, 1H), 2.87-2.81 (m, 1H), 2.32-2.22 (m, 1H), 2.14 (s, 3H), 1.86 (s, 1H). LCMS: (Method A) 496.1 (M⁺−H), Rt. 2.37 min, 99.27% (Max). HPLC: (Method E) Rt. 5.16 min, 99.30% (Max). Chiral HPLC: (Method C) Rt. 2.06 min, 99.12% (Max).

The following compounds were prepared in analogous manner:

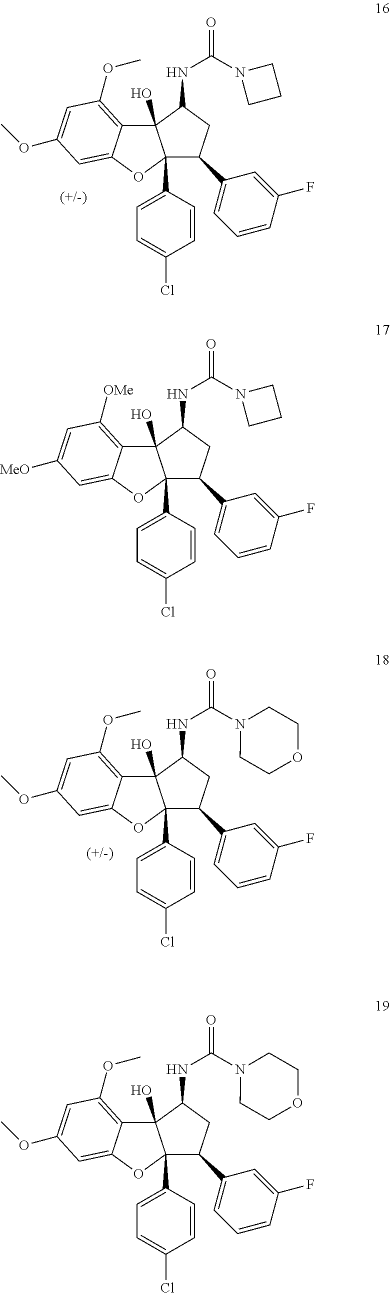

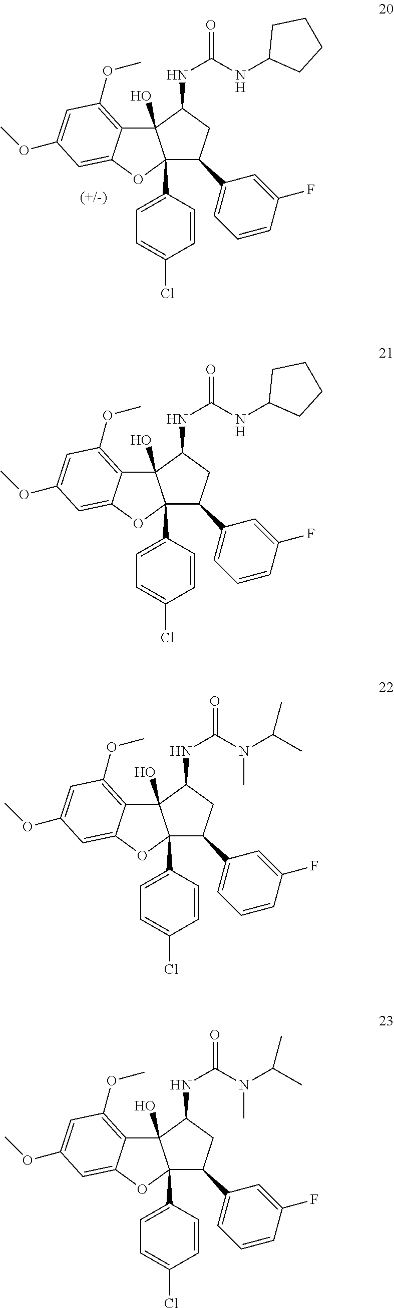 [SMILES: COc1cc(OC)c2c(c1)O[C@@]1(c3ccc(Cl)cc3)[C@H](c3cccc(F)c3)C[C@H](NC(=O)NC3CCCC3)[C@@]21O ; COc1cc(OC)c2c(c1)O[C@@]1(c3ccc(Cl)cc3)[C@H](c3cccc(F)c3)C[C@H](NC(=O)NC3CCCC3)[C@@]21O ; COc1cc(OC)c2c(c1)O[C@@]1(c3ccc(Cl)cc3)[C@H](c3cccc(F)c3)C[C@H](NC(=O)N(C)C(C)C)[C@@]21O ; COc1cc(OC)c2c(c1)O[C@@]1(c3ccc(Cl)cc3)[C@H](c3cccc(F)c3)C[C@H](NC(=O)N(C)C(C)C)[C@@]21O]

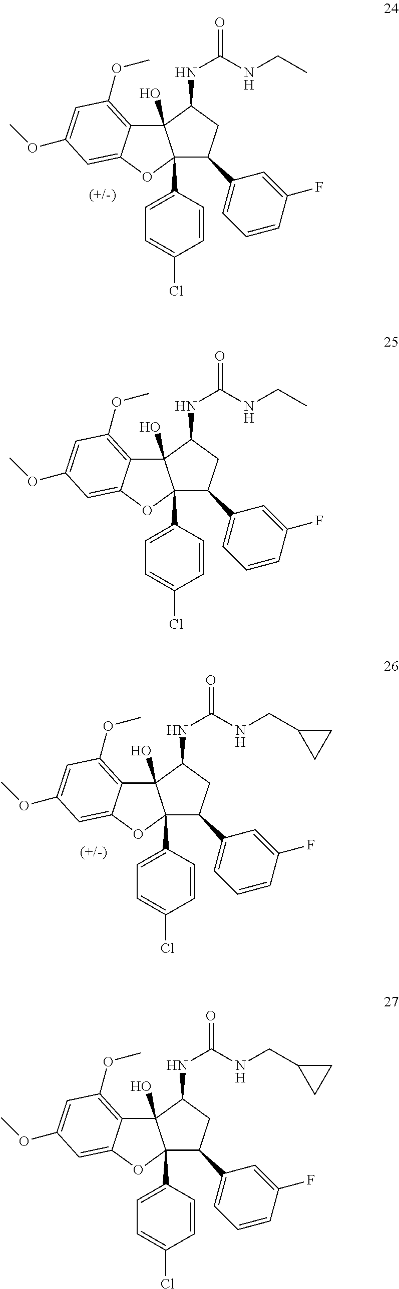 [SMILES: CCNC(=O)N[C@H]1C[C@@H](c2cccc(F)c2)[C@]2(c3ccc(Cl)cc3)Oc3cc(OC)cc(OC)c3[C@]12O ; CCNC(=O)N[C@H]1C[C@@H](c2cccc(F)c2)[C@]2(c3ccc(Cl)cc3)Oc3cc(OC)cc(OC)c3[C@]12O ; COc1cc(OC)c2c(c1)O[C@@]1(c3ccc(Cl)cc3)[C@H](c3cccc(F)c3)C[C@H](NC(=O)NCC3CC3)[C@@]21O ; COc1cc(OC)c2c(c1)O[C@@]1(c3ccc(Cl)cc3)[C@H](c3cccc(F)c3)C[C@H](NC(=O)NCC3CC3)[C@@]21O]

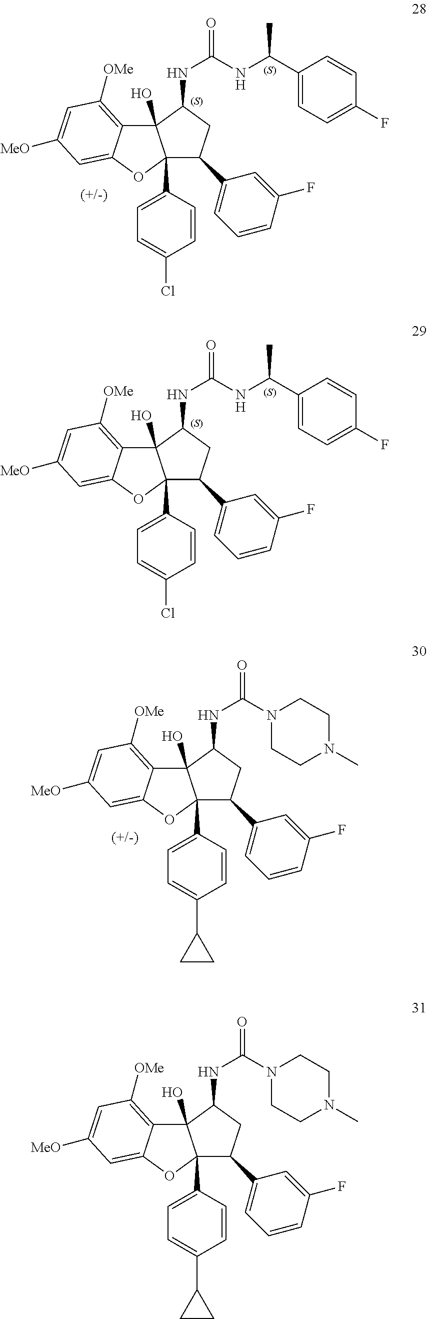

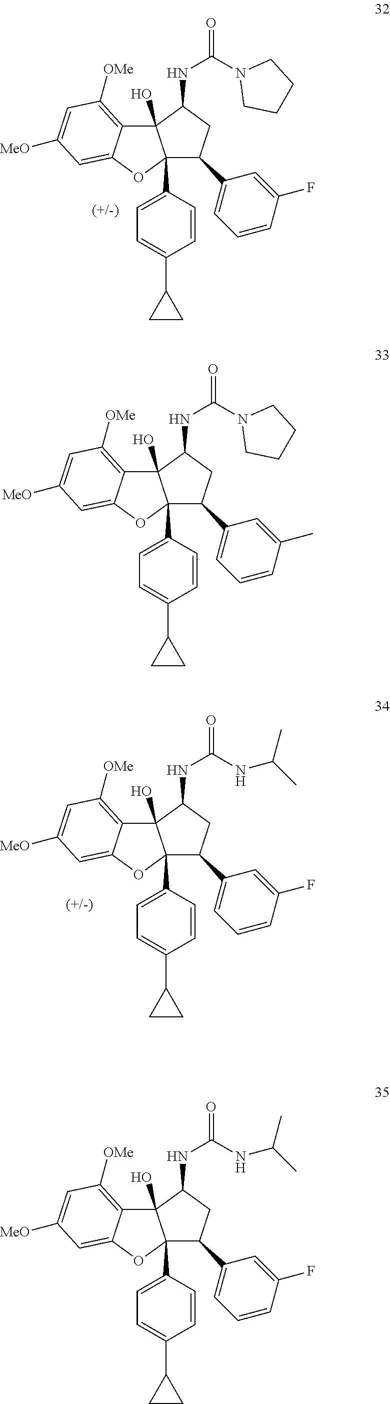 [SMILES: COc1cc(OC)c2c(c1)O[C@@]1(c3ccc(C4CC4)cc3)[C@H](c3cccc(F)c3)C[C@H](NC(=O)N3CCCC3)[C@@]21O ; COc1cc(OC)c2c(c1)O[C@@]1(c3ccc(C4CC4)cc3)[C@H](c3cccc(C)c3)C[C@H](NC(=O)N3CCCC3)[C@@]21O ; COc1cc(OC)c2c(c1)O[C@@]1(c3ccc(C4CC4)cc3)[C@H](c3cccc(F)c3)C[C@H](NC(=O)NC(C)C)[C@@]21O ; COc1cc(OC)c2c(c1)O[C@@]1(c3ccc(C4CC4)cc3)[C@H](c3cccc(F)c3)C[C@H](NC(=O)NC(C)C)[C@@]21O]

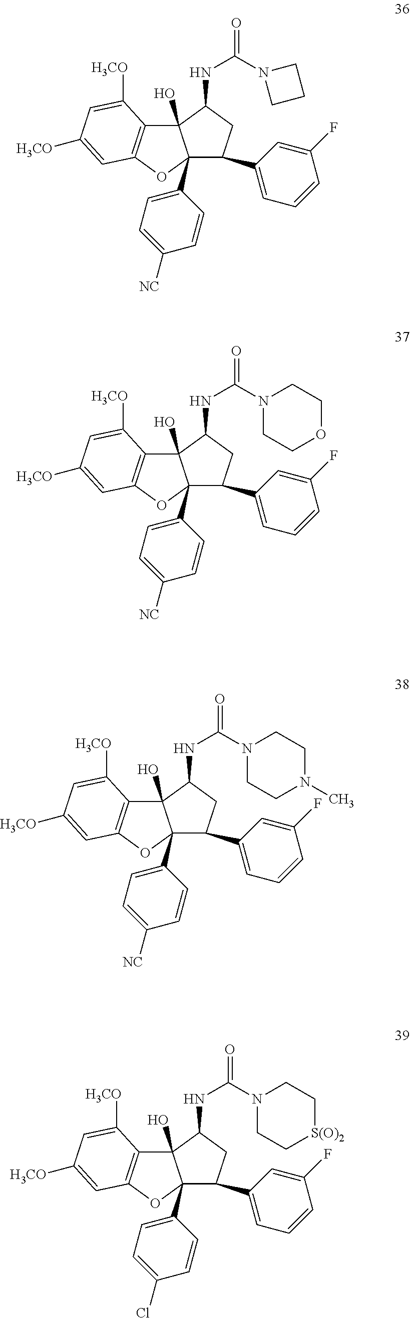

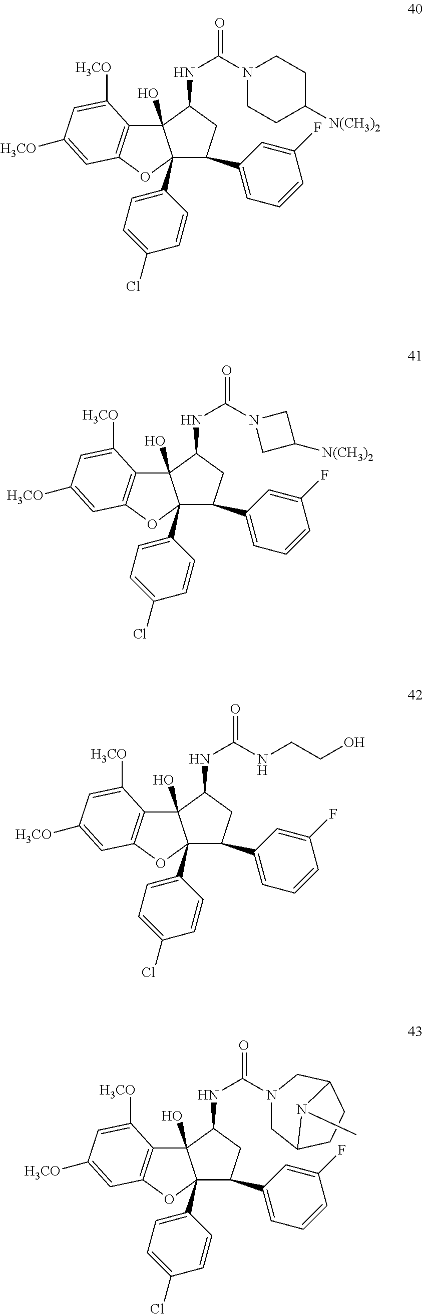 [SMILES: COc1cc(OC)c2c(c1)O[C@@]1(c3ccc(Cl)cc3)[C@H](c3cccc(F)c3)C[C@H](NC(=O)N3CCC(N(C)C)CC3)[C@@]21O ; COc1cc(OC)c2c(c1)O[C@@]1(c3ccc(Cl)cc3)[C@H](c3cccc(F)c3)C[C@H](NC(=O)N3CC(N(C)C)C3)[C@@]21O ; COc1cc(OC)c2c(c1)O[C@@]1(c3ccc(Cl)cc3)[C@H](c3cccc(F)c3)C[C@H](NC(=O)NCCO)[C@@]21O ; COc1cc(OC)c2c(c1)O[C@@]1(c3ccc(Cl)cc3)[C@H](c3cccc(F)c3)C[C@H](NC(=O)N3CC4CCC(C3)N4C)[C@@]21O]

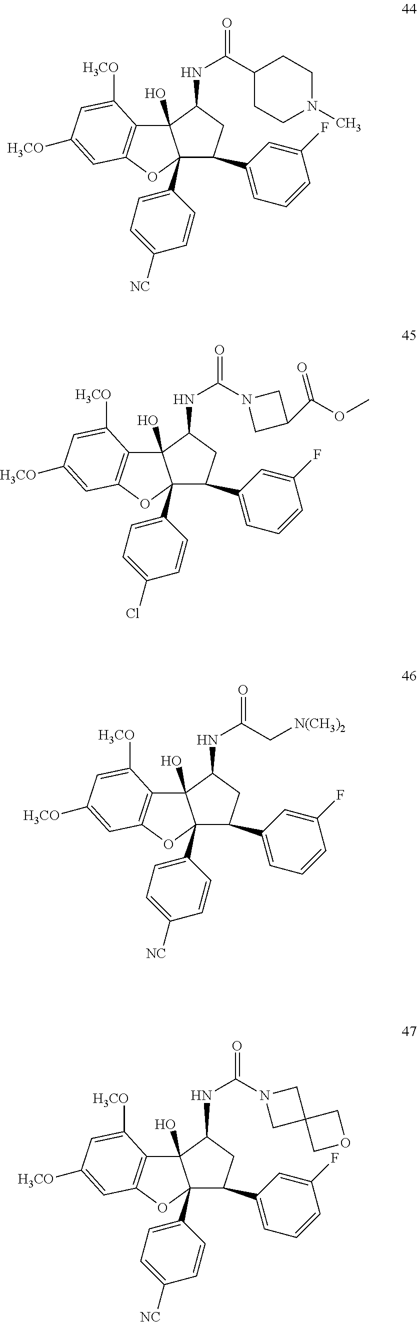 [SMILES: COc1cc(OC)c2c(c1)O[C@@]1(c3ccc(C#N)cc3)[C@H](c3cccc(F)c3)C[C@H](NC(=O)C3CCN(C)CC3)[C@@]21O ; COC(=O)C1CN(C(=O)N[C@H]2C[C@@H](c3cccc(F)c3)[C@]3(c4ccc(Cl)cc4)Oc4cc(OC)cc(OC)c4[C@]23O)C1 ; COc1cc(OC)c2c(c1)O[C@@]1(c3ccc(C#N)cc3)[C@H](c3cccc(F)c3)C[C@H](NC(=O)CN(C)C)[C@@]21O ; COc1cc(OC)c2c(c1)O[C@@]1(c3ccc(C#N)cc3)[C@H](c3cccc(F)c3)C[C@H](NC(=O)N3CC4(COC4)C3)[C@@]21O]

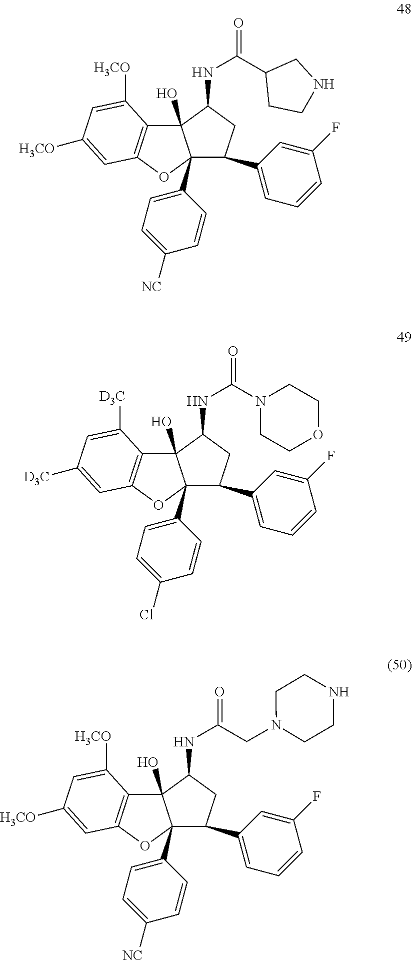 [SMILES: COc1cc(OC)c2c(c1)O[C@@]1(c3ccc(C#N)cc3)[C@H](c3cccc(F)c3)C[C@H](NC(=O)C3CCNC3)[C@@]21O ; [2H]C([2H])([2H])c1cc2c(c(C([2H])([2H])[2H])c1)[C@]1(O)[C@@H](NC(=O)N3CCOCC3)C[C@@H](c3cccc(F)c3)[C@]1(c1ccc(Cl)cc1)O2 ; COc1cc(OC)c2c(c1)O[C@@]1(c3ccc(C#N)cc3)[C@H](c3cccc(F)c3)C[C@H](NC(=O)CN3CCNCC3)[C@@]21O]

### Comparative Example C1: 3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-1,1-dimethylurea (Syn racemic; C1)

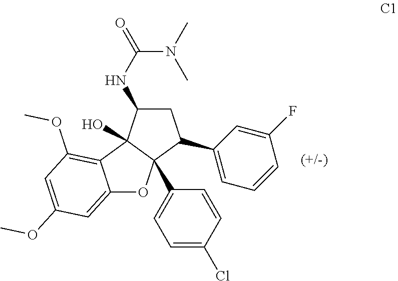

To a stirred solution of (1S,3S,3aR,8bS)-1-amino-3a-(4-chlorophenyl)-3-(3-fluorophenyl)-6,8-dimethoxy-1,2,3,3a-tetrahydro-8bH-cyclopenta[b]benzofuran-8b-ol (0.15 g, 0.33 mmol) in dry DCM (5 mL) at 0° C., DIPEA (51 mg, 0.39 mmol) and DMAP (3 mg) were added. Then dimethyl carbonyl chloride (39 mg, 0.36 mmol) was added at 0° C. and the resulting mixture was stirred 16 h at RT. After completion of the reaction (monitored by TLC), the reaction mixture was quenched with water (25 mL) and the aqueous layer was extracted with DCM (2×50 mL). The combined organic layer was washed with water (20 mL), brine solution (20 mL) and dried over anhydrous Na₂SO₄. The organic part was concentrated under vacuum and the resulting crude was purified by Isolera column chromatography (eluent: 70-80% EtOAc/pet ether; silica gel: 230-400 mesh) to afford the title compound as Syn racemic. Yield: 37% (65 mg, off white solid). ¹H NMR (400 MHz, CDCl₃): δ 7.18-7.06 (m, 5H), 6.90-6.84 (m, 2H), 6.80-6.75 (m, 1H), 6.25 (d, J=2.0 Hz, 1H), 6.07 (d, J=1.6 Hz, 1H), 5.73 (d, J=6.0 Hz, 1H), 4.59-4.57 (m, 1H), 3.85 (s, 3H), 3.78 (s, 3H), 3.77-3.72 (m, 1H), 3.03 (s, 6H), 2.85-2.78 (m, 1H), 2.40-2.30 (m, 1H), 1.99 (s, 1H). LCMS: (Method A) 525.2 (M⁺−H), Rt. 2.43 min, 99.55% (Max). HPLC: (Method C) Rt. 10.59 min, 97.16% (Max).

### Comparative example C2 and C3: 3-((1S,3R,3aR,8bS)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-1,1-dimethylurea & 3-((1R,3R,3aS,8bR)-3a-(4-chlorophenyl)-3-(3-fluorophenyl)-8b-hydroxy-6,8-dimethoxy-2,3,3a,8b-tetrahydro-1H-cyclopenta[b]benzofuran-1-yl)-1,1-dimethylurea (C2 and C3)

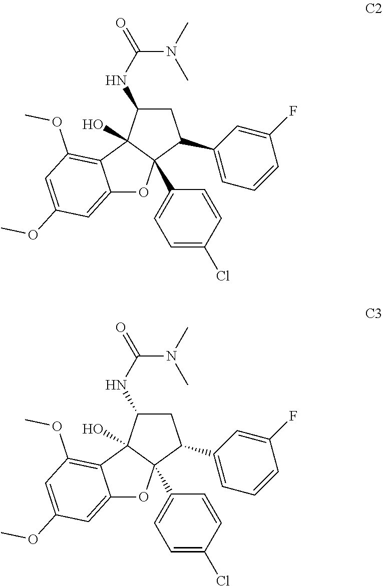

The enantiomers of syn-racemic compound (62 mg; Cl) was separated by SFC (Method A). The first eluting peak was concentrated under vacuum at 40° C. to afford the title compound C2 of chiral purity of 100% and the second eluting peak afforded the title compound C3 of chiral purity 99.63%.

Analytical data of C2: Yield: 15% (9.2 mg, off white solid). ¹H NMR (400 MHz, CDCl₃): δ 7.18-7.07 (m, 5H), 6.90-6.87 (m, 2H), 6.80-6.75 (m, 1H), 6.25 (d, J=1.6 Hz, 1H), 6.07 (d, J=2.0 Hz, 1H), 5.73 (d, J=6.0 Hz, 1H), 4.63-4.58 (m, 1H), 3.85 (s, 3H), 3.78 (s, 3H), 3.76-3.72 (m, 1H), 3.03 (s, 6H), 2.86-2.80 (m, 1H), 2.40-2.30 (m, 1H), 1.99 (s, 1H). LCMS: (Method A) 525.2 (M⁺−H), Rt. 2.41 min, 99.99% (Max). HPLC: (Method B) Rt. 5.22 min, 99.85% (Max). Chiral HPLC: (Method A) Rt. 1.79 min, 100% (Max).

Analytical data of C3: Yield: 15% (9 mg, off white solid). ¹H NMR (400 MHz, CDCl₃): δ 7.18-7.08 (m, 5H), 6.90-6.84 (m, 2H), 6.78-6.78 (m, 1H), 6.25 (d, J=2.0 Hz, 1H), 6.07 (d, J=2.0 Hz, 1H), 5.73 (d, J=6.0 Hz, 1H), 4.62-4.60 (m, 1H), 3.85 (s, 3H), 3.78 (s, 3H), 3.76-3.72 (m, 1H), 3.03 (s, 6H), 2.85-2.82 (m, 1H), 2.37-2.33 (m, 1H), 1.97 (s, 1H). LCMS: (Method A) 525.2 (M⁺−H), Rt. 2.41 min, 99.69% (Max). HPLC: (Method B) Rt. 5.22 min, 99.82% (Max). Chiral HPLC: (Method A) Rt. 3.42 min, 99.63% (Max).

Table A lists data regarding biochemical and cell based activity as well as drug metabolism and pharmacokinetics (DMPK).

TABLE A

| | Biochemical and cell based activity | | In vitro DMPK |
|---|---|---|---|
| | KRAS G12V | | CLi Human |
| Compound | (IC50) nM | elF4A1 (IC50) nM | (μL/min/mg) |
| C2 | 55 | 63 | 290 |
| 2 | 79 | 84 | 5.5 |
| 5 | 95 | 60 | |
| 8 | 78 | 120 | 31 |
| 11 | 94 | 75 | |
| 19 | 58 | 80 | |
| 36 | 24 | 25 | |
| 37 | 19 | 24 | 64 |
| 38 | 21 | 21 | |
| 39 | 32 | 39 | |
| 40 | 97 | 143 | |
| 41 | 50 | 55 | |
| 42 | 48 | 54 | |
| 43 | 57 | 80 | |
| 47 | 41 | 22 | |

1. A compound of formula (I) 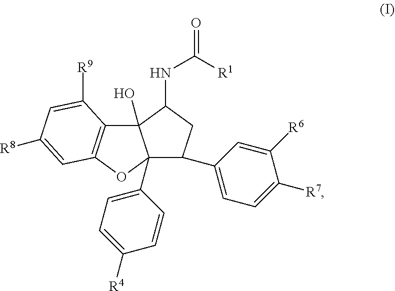
or a pharmaceutically acceptable salt thereof, wherein R¹ is selected from C₁-C₄ alkyl, wherein alkyl is unsubstituted or substituted by 1, 2 or 3 substituents R^{a};
C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2, 3, 4 or 5 identical or different radicals R^{e} and wherein the heterocyloalkyl is connected to the remaining molecule via a carbon atom;
NR²R³, wherein R² and R³ independently from each other are selected from hydrogen, C₁-C₄ alkyl, C₃-C₇ cycloalkyl and C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents R^{h} and wherein alkyl is unsubstituted or substituted by 1, 2 or 3 substituents R^{a}, wherein cycloalkyl is unsubstituted or substituted by 1, 2 or 3 substituents R^{b};
R² and R³ together with the nitrogen atom, which they are attached to, form a 3-, 4-, 5-, 6-, or 7-membered saturated or partly unsaturated heterocyclic ring, wherein the heterocyclic ring has 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{d};
R² and R³ together with the nitrogen atom, which they are attached to, form a saturated or partly unsaturated bi-, tri- or tetracyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{f};
R² and R³ together with the nitrogen atom, which they are attached to, form a saturated or partly unsaturated spiro moiety, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2, 3, 4 or 5 identical or different radicals R^{g};
R^{a} is selected from halogen, OH, C₃-C₇ cycloalkyl, C₁-C₃ alkoxy, phenyl, NR^{5a}R^{5b}, C₁-C₄-alkylsulfonyl, C₃-C₇ heterocyloalkyl, comprising 1, 2 or 3 identical or different heteroatoms or heteroatom-containing groups as ring members, selected from N, NR^{c}, O, S, SO and SO₂, and wherein the heterocyloalkyl is unsubstituted or substituted by 1, 2 or 3 substituents selected from halogen, C₁-C₄-alkyl, C₁-C₄-haloalkyl and wherein C₃-C₇ cycloalkyl and phenyl is unsubstituted or substituted by 1, 2 or 3 substituents selected from F, Cl, Br and OH;
R^{b} is selected from halogen, OH and C₁-C₃ alkoxy;
R^{c} is selected from hydrogen, C₁-C₄-alkyl, C₃-C₇ cycloalkyl, C₁-C₄-haloalkyl, C₁-C₄-cyanoalkyl, carbonyloxy-C₁-C₄-alkyl and C₁-C₄-hydroxyalkyl;
R^{d} is selected from halogen, C₁-C₄-alkyl, C₁-C₄-haloalkyl, C₁-C₄-hydroxyalkyl, C₁-C₄-alkoxy, C₁-C₄-haloalkoxy, carboxy, carbonyloxy-C₁-C₄-alkyl and NR^{5a}R^{5b};
R^{e} is selected from halogen, C₁-C₄-alkyl, C₁-C₄-haloalkyl, C₁-C₄-hydroxyalkyl, C₁-C₄-alkoxy, carbonyloxy-C₁-C₄-alkyl and C₁-C₄-haloalkoxy;
R^{f} is selected from halogen, C₁-C₄-alkyl, C₁-C₄-haloalkyl, C₁-C₄-hydroxyalkyl, C₁-C₄-alkoxy, C₁-C₄-haloalkoxy and NR^{5a}R^{5b};
R^{g} is selected from halogen, C₁-C₄-alkyl, C₁-C₄-haloalkyl, C₁-C₄-hydroxyalkyl, C₁-C₄-alkoxy and C₁-C₄-haloalkoxy;
R^{h} is selected from halogen, C₁-C₄-alkyl, C₁-C₄-haloalkyl, C₁-C₄-hydroxyalkyl, C₁-C₄-alkoxy and C₁-C₄-haloalkoxy;
R⁴ is selected from Cl, CN and C₃-C₇ cycloalkyl;
R^{5a} and R^{5b} independently of each other are selected from hydrogen, C₁-C₄-alkyl and C₃-C₇cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
with the proviso that the following compounds are excluded:
R¹ is NH₂, R⁴ is Cl, R⁶ is F, R⁷ is hydrogen, R³ is OCH₃, R⁹ is OCH₃,
R¹ is N(CH₃)₂, R⁴ is Cl, R⁶ is F, R⁷ is hydrogen, R³ is OCH₃, R⁹ is OCH₃,
R¹ is CH₂N(CH₃)₂, R⁴ is Cl, R⁶ is F, R⁷ is hydrogen, R³ is OCH₃, R⁹ is OCH₃.

2. (canceled)

3. The compound of formula (I) according to claim 1, which is a compound of formula (I.a) or an enantiomeric mixture comprising the compounds of formula (I.a) and (I.b) or the pharmaceutically acceptable salts of compound of formula (I.a) or the enantiomeric mixture comprising the compounds of formula (I.a) and (I.b) 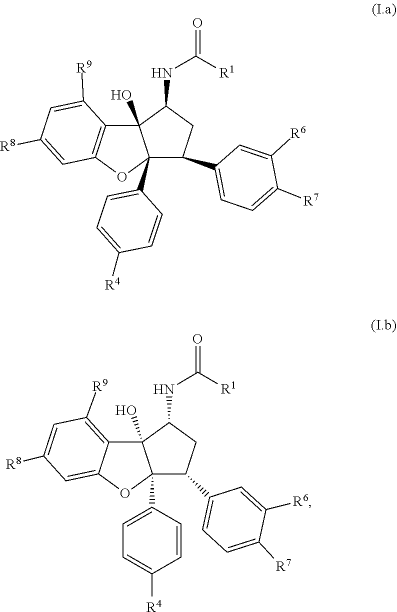
wherein R¹, R⁴, R⁶, R⁷, R⁸, R⁹ have the same meanings as defined in claim 1.

4. The compound of formula (I) according to claim 1, which is a racemic mixture (I.a′) or the pharmaceutically acceptable salt thereof, 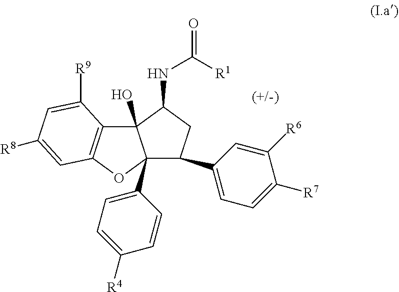
wherein R¹, R⁴, R⁶, R⁷, R⁸, R⁹ have the same meanings as defined in claim 1.

5. The compound of formula (I) according to claim 31, being a mixture of (I.a) and (I.b) or a mixture of the pharmaceutically acceptable salts thereof, wherein the enantiomer excess (ee) of the enantiomer of formula (I.a) is at least 20%, preferably at least 50%, in particular at least 80%, especially at least 99%.

6. The compound according to claim 1, or a pharmaceutically acceptable salt thereof, wherein R⁶ is F and R⁷ is hydrogen.

7. The compound according to claim 1, or a pharmaceutically acceptable salt thereof, wherein R¹ is selected from
C₁-C₄ alkyl, which is unsubstituted or substituted by 1, 2 or 3 substituents R^{a}; or
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1, 2 or 3 identical or different radicals R^{e} and wherein the heterocyloalkyl is connected to the remaining molecule via a carbon atom; or
NR²R³, wherein R² and R³ independently from each other are selected from hydrogen, C₁-C₄ alkyl and C₃-C₆ cycloalkyl, wherein alkyl is unsubstituted or substituted by 1, 2 or 3 substituents R^{a}, wherein cycloalkyl is unsubstituted or substituted by 1, 2 or 3 substituents R^{b}; or
R² and R³ together with the nitrogen atom, which they are attached to, form a 3-, 4-, 5-, or 6-membered saturated or partly unsaturated heterocyclic ring, wherein the heterocyclic ring has 1, 2 or 3 heteroatoms or heteroatom-containing groups as ring members, selected from N, NR^{c}, S, SO and SO₂, wherein the heterocyclic ring is unsubstituted or substituted by 1, 2, 3, 4 or 5 identical or different radicals R^{d}; or
R² and R³ together with the nitrogen atom, which they are attached to, form a saturated or partly unsaturated bi- or tricyclic ring system, comprising 1, 2 or 3 identical or different heteroatoms or heteroatom-containing groups as ring members, selected from N, NR^{c}, O, S, SO and SO₂, and wherein the heterocyclic ring is unsubstituted or substituted by 1, 2 or 3 identical or different radicals R^{f}; or
R² and R³ together with the nitrogen atom, which they are attached to, form a saturated or partly unsaturated spiro moiety, comprising 1 or 2 identical or different heteroatoms or heteroatom-containing groups as ring members, selected from N, NR^{c} and O and wherein the heterocyclic ring is unsubstituted or substituted by 1, 2 or 3 identical or different radicals R^{g};
R⁴, R^{a}, R^{b}, R^{c},R^{d}, R^{e}, R^{f} and R^{g} have one of the meanings as defined in claim 1 or 2.

8. The compound according to claim 1, or a pharmaceutically acceptable salt thereof, wherein R¹ is selected from
NR²R³, wherein R² and R³ independently from each other are selected from hydrogen, C₁-C₃ alkyl and C₃-C₆ cycloalkyl, wherein alkyl is unsubstituted or substituted by 1 or 2 substituents R^{a}, wherein cycloalkyl is unsubstituted or substituted by 1 or 2 substituents R^{b}, preferably R² and R³ independently from each other are selected from hydrogen, C₂-C₃ alkyl, which is unsubstituted and C₃-C₆ cycloalkyl, which is unsubstituted; or
R² and R³ together with the nitrogen atom, which they are attached to, form a 5- or 6-membered saturated or partly unsaturated heterocyclic ring, comprising 1 or 2 heteroatoms or heteroatom-containing groups as ring members, selected from N, NR^{c}, SO₂ and O, wherein R^{c} is selected from hydrogen, C₁-C₄-alkyl, wherein the heterocyclic ring is unsubstituted or substituted by 1 or 2 identical or different radicals selected from C₁-C₄-alkyl, C₁-C₄-hydroxyalkyl, C₁-C₄-alkoxy, NH₂, N(C₁-C₂ alkyl)₂, NH(C₁-C₂ alkyl) and carbonyloxy-C₁-C₄-alkyl, preferably R² and R³ together with the nitrogen atom, which they are attached to, form a pyrrolidine ring, piperazine ring, acetidin ring or morpholin ring, wherein the pyrrolidine ring, piperazine ring, acetidin ring, or morpholin ring is unsubstituted or substituted by 1 or 2 substituents selected from C₁-C₄-alkyl, C₁-C₄-hydroxyalkyl, C₁-C₄-alkoxy, NH₂, N(C₁-C₂ alkyl)₂, NH(C₁-C₂ alkyl) and carbonyloxy-C₁-C₄-alkyl; or
C₃-C₇ heterocyloalkyl, comprising 1 or 2 identical or different heteroatoms or heteroatom-containing groups as ring members, selected from NR^{c} and O, wherein the heterocyloalkyl is unsubstituted or substituted by 1 or 2 identical or different radicals R^{e} and wherein the heterocyloalkyl is connected to the remaining molecule via a carbon atom; or
R² and R³ together with the nitrogen atom, which they are attached to, form a saturated or partly unsaturated bicyclic ring system, comprising 1 or 2 identical or different heteroatoms or heteroatom-containing groups as ring members, selected from N or NR^{c}, preferably R² and R³ together with the nitrogen atom, which they are attached to, form a 8-methyl-3,8-diazabicyclo[3.2.1]octan-3-yl ring system; or
R² and R³ together with the nitrogen atom, which they are attached to, form a saturated or partly unsaturated spiro moiety, comprising 2 identical or different heteroatoms or heteroatom-containing groups as ring members, selected from N, NR^{c} and O, preferably R² and R³ together with the nitrogen atom, which they are attached to, form a 2-oxa-6-azaspiro[3.3]heptan-6-yl spiro compound.

9. The compound according to claim 1, or a pharmaceutically acceptable salt thereof, wherein R¹ is selected from C₅-C₇ heterocyloalkyl, comprising 1 or 2 heteroatoms or heteroatom-containing groups as ring members, selected from NR^{c} and S, wherein the heterocyloalkyl is connected to the remaining molecule via a carbon atom, preferably R¹ is selected from pyrrolidinyl and piperidinyl.

10. The compound according to claim 1, or a pharmaceutically acceptable salt thereof, wherein R¹ is selected from methyl, 4-methyl-piperazin-1-yl, pyrrolidin-1-yl, N,N-diethylamino, N-isopropylamino, N-ethylamino, N,N-methyl-isopropylamino, acetidin-1-yl, morpholin-4-yl, N-cyclopentylamino, [1-(4-fluorophenyl)ethyl]amino, (cyclopropylmethyl)amino, 8-methyl-3,8-diazabicyclo[3.2.1]octan-3-yl, 1-methylpiperidin-4-yl, thiomorpholine-4-yl-1,1-dioxide, 3-(dimethylamino)azetidin-1-yl, 4-(dimethylamino)piperidin-1-yl, N-ethan-1-ol-amino, azetidine-3-carbonyloxymetyl, N,N-dimethylamino-methyl, 2-oxa-6-azaspiro[3.3]heptan-6-yl and pyrrolidine-3-yl.

11. The compound according to claim 1, selected from compounds of the formulae A, B, C, D, E, F, G, H, I, J, K, L, M, N, O, P, Q, R, S, T, U, V and the mixture of the each of compounds A to V with its respective enantiomer, 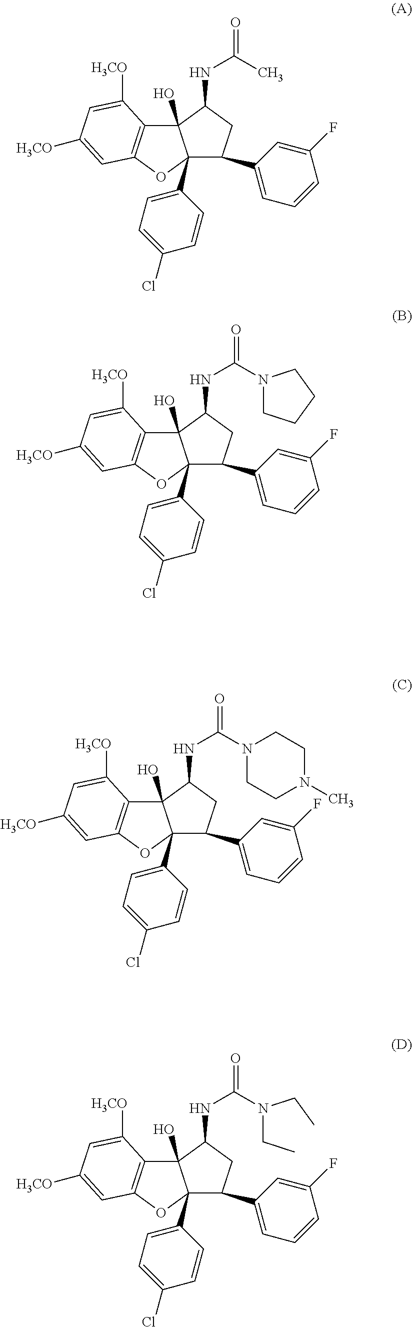 [SMILES: COc1cc(OC)c2c(c1)O[C@@]1(c3ccc(Cl)cc3)[C@H](c3cccc(F)c3)C[C@H](NC(C)=O)[C@@]21O ; COc1cc(OC)c2c(c1)O[C@@]1(c3ccc(Cl)cc3)[C@H](c3cccc(F)c3)C[C@H](NC(=O)N3CCCC3)[C@@]21O ; COc1cc(OC)c2c(c1)O[C@@]1(c3ccc(Cl)cc3)[C@H](c3cccc(F)c3)C[C@H](NC(=O)N3CCN(C)CC3)[C@@]21O ; CCN(CC)C(=O)N[C@H]1C[C@@H](c2cccc(F)c2)[C@]2(c3ccc(Cl)cc3)Oc3cc(OC)cc(OC)c3[C@]12O] 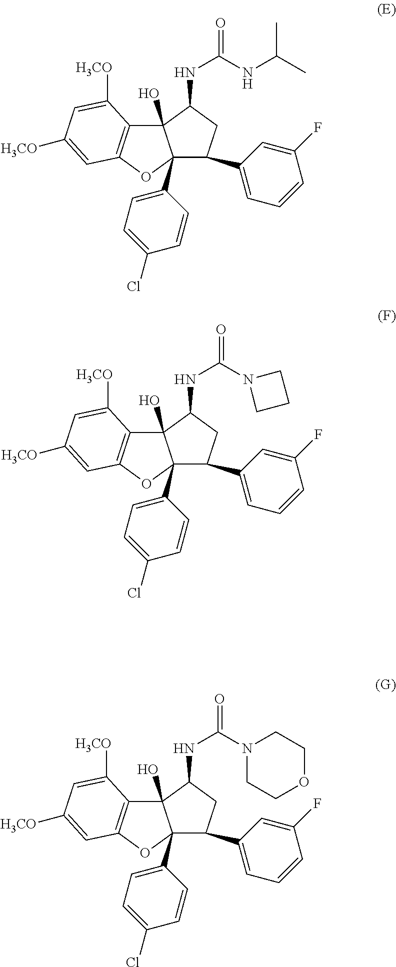 [SMILES: COc1cc(OC)c2c(c1)O[C@@]1(c3ccc(Cl)cc3)[C@H](c3cccc(F)c3)C[C@H](NC(=O)NC(C)C)[C@@]21O ; COc1cc(OC)c2c(c1)O[C@@]1(c3ccc(Cl)cc3)[C@H](c3cccc(F)c3)C[C@H](NC(=O)N3CCC3)[C@@]21O ; COc1cc(OC)c2c(c1)O[C@@]1(c3ccc(Cl)cc3)[C@H](c3cccc(F)c3)C[C@H](NC(=O)N3CCOCC3)[C@@]21O] 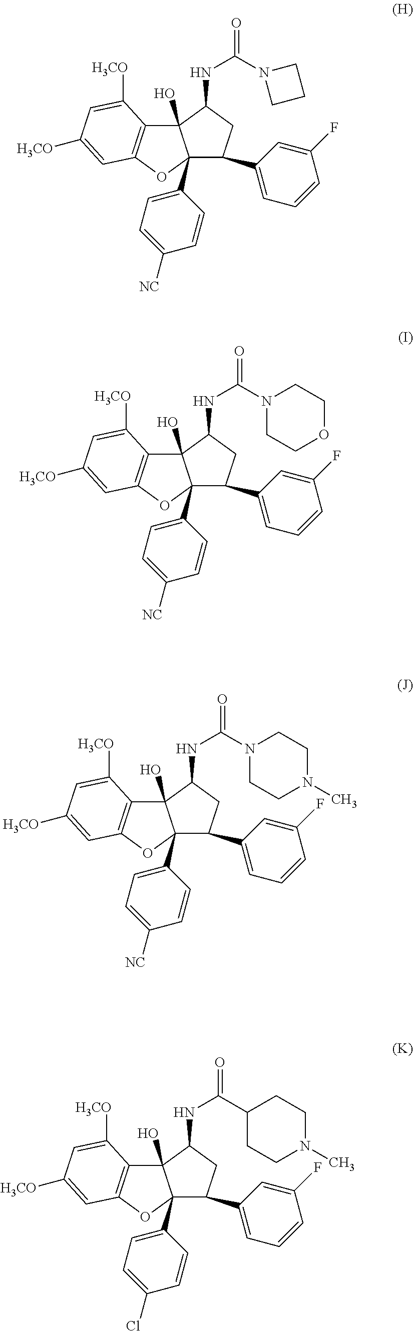 [SMILES: COc1cc(OC)c2c(c1)O[C@@]1(c3ccc(C#N)cc3)[C@H](c3cccc(F)c3)C[C@H](NC(=O)N3CCC3)[C@@]21O ; COc1cc(OC)c2c(c1)O[C@@]1(c3ccc(C#N)cc3)[C@H](c3cccc(F)c3)C[C@H](NC(=O)N3CCOCC3)[C@@]21O ; COc1cc(OC)c2c(c1)O[C@@]1(c3ccc(C#N)cc3)[C@H](c3cccc(F)c3)C[C@H](NC(=O)N3CCN(C)CC3)[C@@]21O ; COc1cc(OC)c2c(c1)O[C@@]1(c3ccc(Cl)cc3)[C@H](c3cccc(F)c3)C[C@H](NC(=O)C3CCN(C)CC3)[C@@]21O] 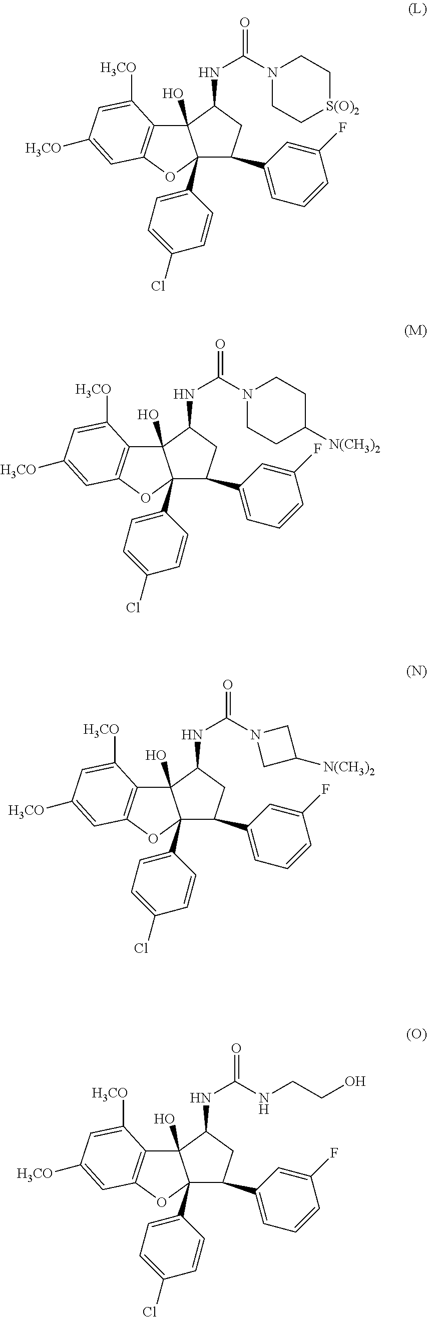 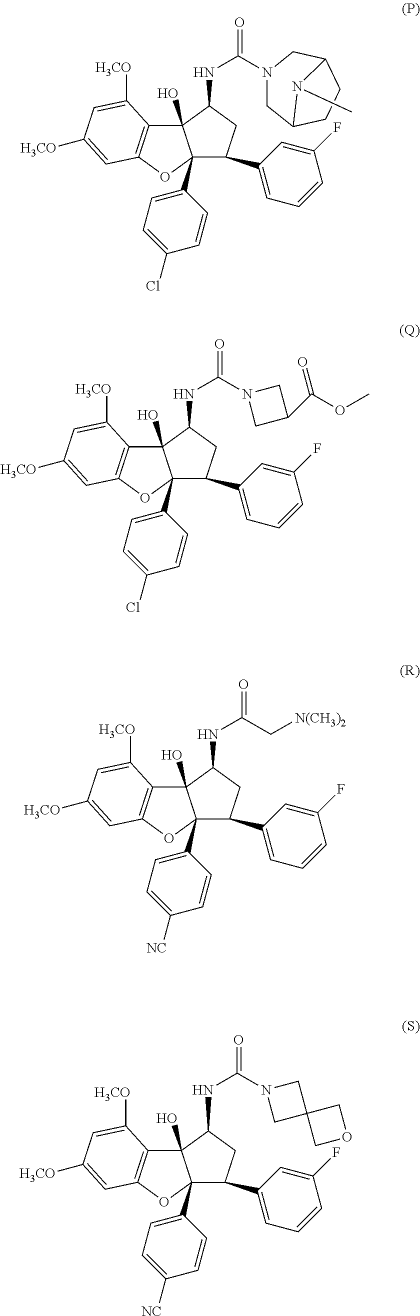 [SMILES: COc1cc(OC)c2c(c1)O[C@@]1(c3ccc(Cl)cc3)[C@H](c3cccc(F)c3)C[C@H](NC(=O)N3CC4CCC(C3)N4C)[C@@]21O ; COC(=O)C1CN(C(=O)N[C@H]2C[C@@H](c3cccc(F)c3)[C@]3(c4ccc(Cl)cc4)Oc4cc(OC)cc(OC)c4[C@]23O)C1 ; COc1cc(OC)c2c(c1)O[C@@]1(c3ccc(C#N)cc3)[C@H](c3cccc(F)c3)C[C@H](NC(=O)CN(C)C)[C@@]21O ; COc1cc(OC)c2c(c1)O[C@@]1(c3ccc(C#N)cc3)[C@H](c3cccc(F)c3)C[C@H](NC(=O)N3CC4(COC4)C3)[C@@]21O] 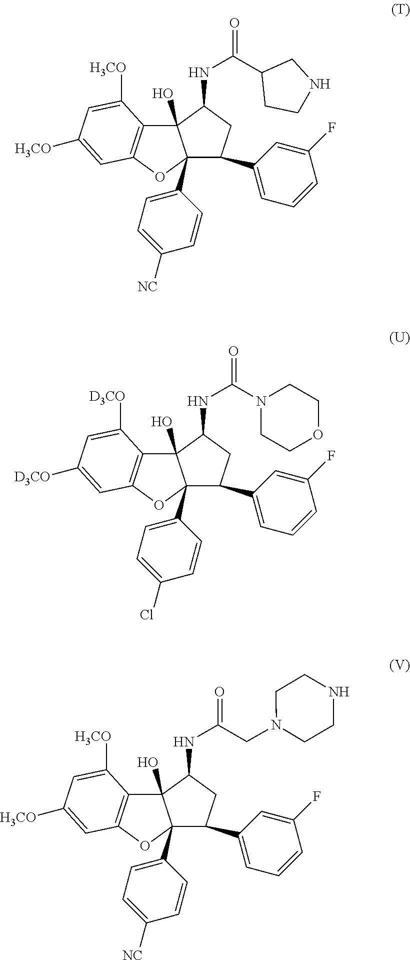
or mixtures selected from two or more compounds (A) to (V) and the enantiomers thereof.

12. (canceled)

13. (canceled)

14. (canceled)

15. (canceled)

16. (canceled)

17. (canceled)

18. (canceled)

19. (canceled)

20. (canceled)

21. A pharmaceutical composition comprising at least one compound of formula (I), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) according to claim 1, an enantiomeric mixture thereof, wherein each of the compounds can be in the form of a pharmaceutically acceptable salt, and a pharmaceutically acceptable carrier.

22. The pharmaceutical composition according to claim 21, comprising additionally a further active substance, preferably selected from chemotherapeutic agents, radiotherapeutic agents, immuno-oncology agents and combinations thereof.

23. The pharmaceutical composition according to claim 21 for use in the prophylaxis and/or treatment of proliferative disorders.

24. (canceled)

25. A method of inhibiting growth, proliferation, or metastasis of cancer cells in a subject in need thereof, said method comprising administering to the subject a therapeutically effective amount of at least one compound of formula (I), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) according to claim 1, an enantiomeric mixture thereof, wherein each of the compounds can be in the form of a pharmaceutically acceptable salt.

26. The method of claim 25, wherein the cancer is selected from prostate, colon, rectum, pancreas, cervix, stomach, endometrium, brain, liver, bladder, ovary, testis, head, neck, skin (including melanoma and basal carcinoma), mesothelial lining, white blood cell (including lymphoma and leukemia), esophagus, breast, muscle, connective tissue, lung (including small cell lung carcinoma and non-small-cell carcinoma), adrenal gland, thyroid, kidney, or bone; or glioblastoma, mesothelioma, renal cell carcinoma, gastric carcinoma, sarcoma (including Kaposi's sarcoma), choriocarcinoma, cutaneous basocellular carcinoma, Haematological malignancies (including blood, bone marrow and lymph nodes) or testicular seminoma.

27. (canceled)

28. (canceled)

29. A kit containing a formulation comprising: a1) at least one compound of formula (I), in particular a compound selected from formulae (I.a), (I.a′), (A), (B), (C), (D), (E), (F) (G), (H), (I), (J), (K), (L), (M), (N), (O), (P), (Q), (R), (S), (T), (U), (V) according to claim 1, an enantiomeric mixture thereof, wherein each of the compounds can be in the form of a pharmaceutically acceptable salt or a2) a pharmaceutical composition comprising at least one compound of formula (I), in particular a compound selected from formulae (I.a), (I.a′), (A), (B), (C), (D), (E), (F), (G), (H), (I), (J), (K), (L), (M), (N), (O), (P), (Q), (R), (S), (T), (U), (V) according to claim 1, an enantiomeric mixture thereof, wherein each of the compounds can be in the form of a pharmaceutically acceptable and a pharmaceutically acceptable carrier; and b) instructions for dosing of the pharmaceutical composition for the treatment of a disorder in which inhibition of RAS activation or the downstream signaling pathways is effective in treating the disorder, or for the treatment of a disorder in which inhibition of the activity of eIF4A, is effective in treating the disorder.

30. (canceled)

31. A process for the preparation of a compound of the formula (I) 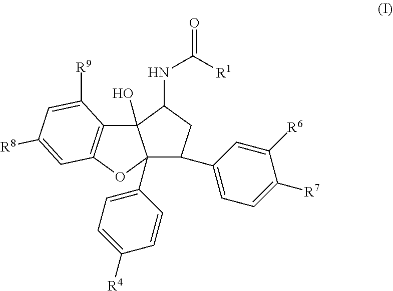
or stereoisomers, tautomers, or pharmaceutically acceptable salts thereof, wherein
R¹ is defined as in claim 1;
R⁴ is selected from Cl, CN and C₃-C₇ cycloalkyl;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R⁸ is selected from OCH₃, OCD₃;
R⁹ is selected from OCH₃, OCD₃;
comprising the steps
a1) providing a compound of the formula (II) 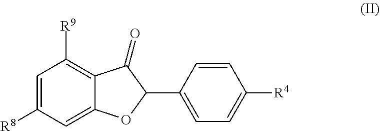
wherein
R⁴ is selected from Cl, CN and C₃-C₇ cycloalkyl,
a2) reacting the compound (II) with a compound (III) 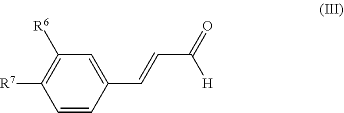
to yield the adduct (IV) 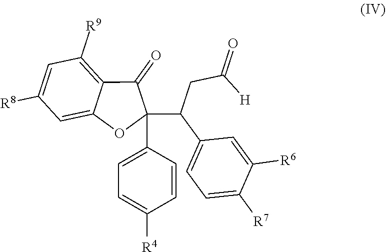
a3) reacting the compound (IV) with trimethylsilylcyanide to give the cyanohydrin silylether (V) 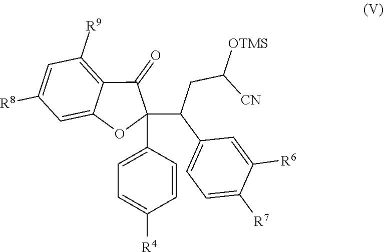
a4) subjecting the compound (V) to a ring formation reaction in the presence of a base to give the compound (VI) 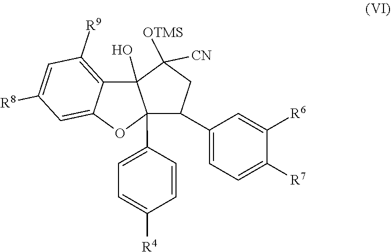
a5) reacting the compound (VI) with tetra-n-butylammonium fluoride to give the compound (VII) 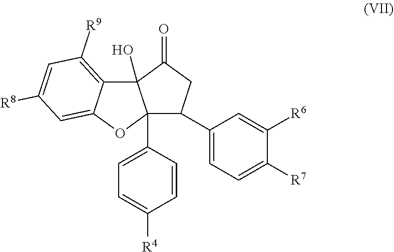
a6) reacting the compound (VII) with methoxyamine hydrochloride to give the oxime compound (VIII) 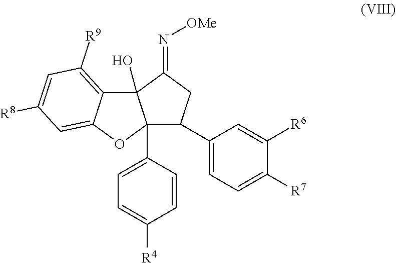
a7) reduction of the oxime compound (VIII) give the amine compound (IX) 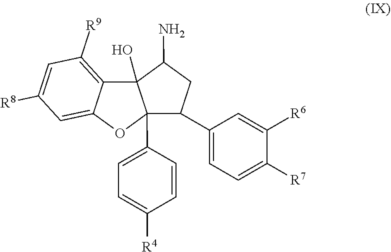
a8.1) subjecting the amine compound (IX) to a reaction with a compound of the formula (X.1)
R¹—C(═O)—X (X.1)
wherein X is a leaving group selected from Cl, Br, O-benzyl, CH₃SO₃ and CF₃SO₃
to give the compound of the formula (I)
or
a8.2) subjecting the amine compound (IX) to a reaction with an isocyanate of the formula (X.2)
R²—N═C═O (X.2)
to give a compound of the formula (I), wherein R¹ is a group NHR², wherein R² is selected from C₁-C₄ alkyl and C₃-C₇ cycloalkyl, wherein alkyl is unsubstituted or substituted by 1, 2 or 3 substituents R^{a}, wherein cycloalkyl is unsubstituted or substituted by 1, 2 or 3 substituents R^{b},
R^{a} is selected from halogen, OH, C₃-C₇ cycloalkyl, C₁-C₃ alkoxy and phenyl, wherein C₃-C₇ cycloalkyl and phenyl is unsubstituted or substituted by 1, 2 or 3 substituents selected from F, Cl, Br and OH,
R^{b} is selected from halogen, OH and C₁-C₃ alkoxy,
a9) optionally subjecting at least one compound, selected from compounds (IV) obtained in step a2), compounds (V) obtained in step a3), compounds (VI) obtained in step a4), compounds (VII) obtained in step a5), compounds (VIII) obtained in step a6), compounds (IX) obtained in step a7), compounds (I) obtained in step a8.1) and compounds (I) obtained in step a8.2), to one or more purification step(s).

32. The process according to claim 31 for the preparation of a compound of the formula (I) 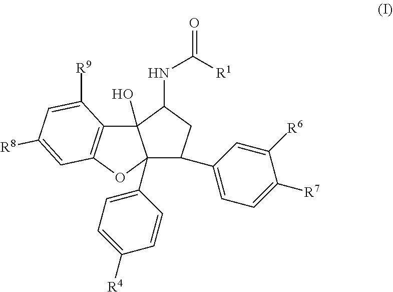
or stereoisomers, tautomers, or pharmaceutically acceptable salts thereof, wherein
R¹ is defined as in claim 1;
R⁴ is ON;
R⁶ is selected from hydrogen and F;
R⁷ is selected from hydrogen and C₁-C₂-alkyl;
with the proviso that, if R⁶ is H, R⁷ is C₁-C₂-alkyl and if R⁶ is F, R⁷ is hydrogen;
R³ is selected from OCH₃, OCD₃;
R⁹ is selected from OCH₃, OCD₃;
comprising the steps
a1) providing a compound of the formula (II) 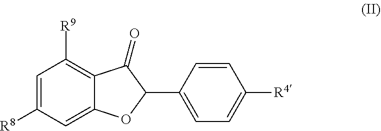
wherein
R^{4′} is selected from halogen, in particular Br,
a2) reacting the compound (II) with a compound (III) 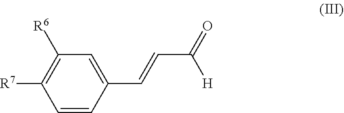
to yield the adduct (IV) 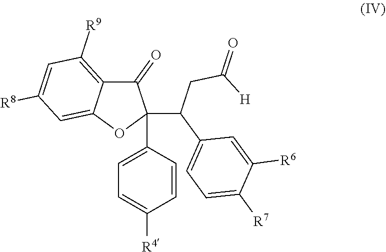
a3) reacting the compound (IV) with trimethylsilylcyanide to give the cyanohydrin silylether (V) 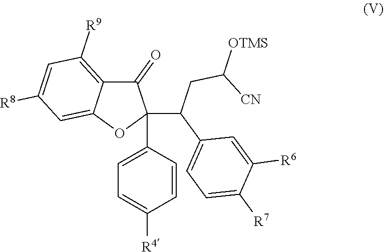
a4) subjecting the compound (V) to a ring formation reaction in the presence of a base to give the compound (VI) 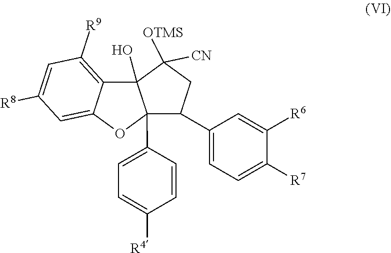
a5) reacting the compound (VI) with tetra-n-butylammonium fluoride to give the compound (VII) 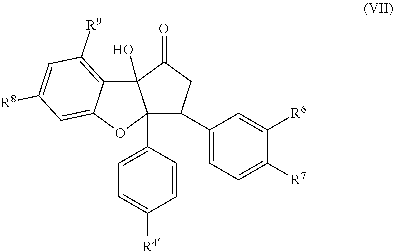
a6) reacting the compound (VII) with methoxyamine hydrochloride to give the oxime compound (VIII) 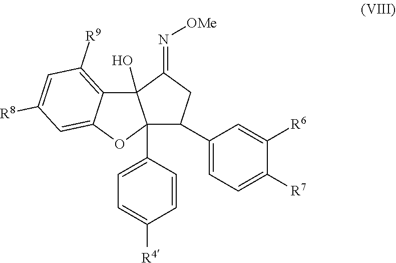
a7) reduction of the oxime compound (VIII) give the amine compound (IX) 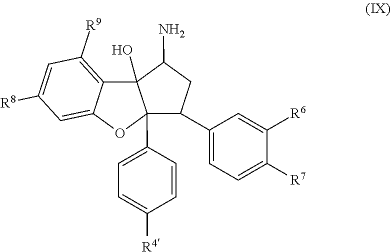
a7.1) reacting the compound (IX) with benzyl carbonochloridate to give the compound (IX′) 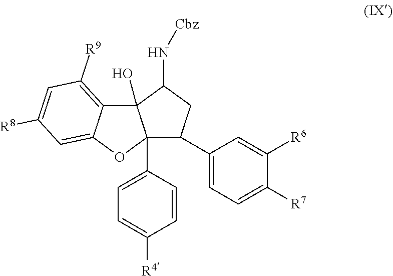
wherein Cbz is benzyloxycarbonyl,
a7.2) reacting the compound (IX′) with dicyanozinc followed by the cleavage of the Cbz group to give the compound (IX″), 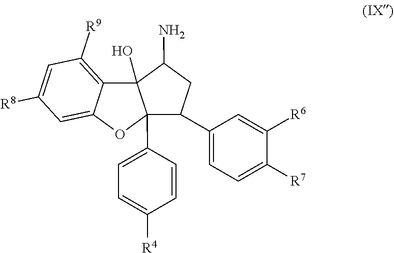
a8.1) subjecting the amine compound (IX″) to a reaction with a compound of the formula (X.1)
R¹—C(═O)—X (X.1)
wherein X is a leaving group selected from Cl, Br, O-benzyl, CH₃SO₃ and CF₃SO₃
to give the compound of the formula (I)
or
a8.2) subjecting the amine compound (IX″) to a reaction with an isocyanate of the formula (X.2)
R²—N═C═O (X.2)
to give a compound of the formula (I), wherein R¹ is a group NHR², wherein R² is selected from C₁-C₄ alkyl and C₃-C₇ cycloalkyl, wherein alkyl is unsubstituted or substituted by 1, 2 or 3 substituents R^{a}, wherein cycloalkyl is unsubstituted or substituted by 1, 2 or 3 substituents R^{b},
R^{a} is selected from halogen, OH, C₃-C₇ cycloalkyl, C₁-C₃ alkoxy and phenyl, wherein C₃-C₇ cycloalkyl and phenyl is unsubstituted or substituted by 1, 2 or 3 substituents selected from F, Cl, Br and OH,
R^{b} is selected from halogen, OH and C₁-C₃ alkoxy,
a9) optionally subjecting at least one compound, selected from compounds (IV) obtained in step a2), compounds (V) obtained in step a3), compounds (VI) obtained in step a4), compounds (VII) obtained in step a5), compounds (VIII) obtained in step a6), compounds (IX) obtained in step a7), compounds (1) obtained in step a8.1) and compounds (1) obtained in step a8.2), to one or more purification step(s).

33. (canceled)

34. A method of treatment and/or prophylaxis of a disease or condition mediated by RAS, eIF4A, or a prohibitin (PHB/2) in a subject in need thereof, comprising
administering to the subject the compound of claim 1,
wherein said disease or condition is the result of any mutation in RAS in said subject that leads to reduced activity of a RAS inhibitor, or
wherein said disease or condition is the result of dysregulation of eIF4A in said subject, or
wherein said disease or condition is the result of overexpression of one or more prohibitins in said subject.
